(19) United States
(12) Patent Application Publication
(10) Pub. No.: US 2022/0348745 A1
(43) Pub. Date: Nov. 3, 2022

(54) Compositions and Methods for Treating a Disorder or Defect in Soft Tissue

(71) Applicant: Drexel University, Philadelphia, PA (US)

(72) Inventors: Michele Marcolongo, Aston, PA (US); Benjamin Jackson, Chadds Ford, PA (US); Sumona Sarkar, Philadelphia, PA (US); Caroline Schauer, Hulmeville, PA (US)

(21) Appl. No.: 17/569,586

(22) Filed: Jan. 6, 2022

Related U.S. Application Data
(63) Continuation of application No. 16/178,371, filed on Nov. 1, 2018, now abandoned.
Application No. 16/178,371 is a continuation of application No. 13/508,466, filed on Nov. 13, 2012, now abandoned.
(60) Provisional application No. 61/259,435, filed on Nov. 9, 2009.

(51) Int. Cl. C08L 5/08; C08B 37/08; C08B 37/00; C08L 5/00; C08L 5/10; C08G 81/00

(52) U.S. Cl. CPC C08L 5/08; C08B 37/003; C08B 37/0063; C08B 37/0069; C08B 37/0072; C08B 37/0075; C08L 5/00; C08L 5/10; C08G 81/00

(57) ABSTRACT

The present invention encompasses methods and compositions for generating a biomimetic proteoglycan. The invention includes methods of treating a disease, disorder, or condition of soft tissue using a biomimetic proteoglycan.

### CROSS-REFERENCE TO RELATED APPLICATIONS

This application is a continuation application of, and claims priority to, U.S. patent application Ser. No. 16/178,371, filed Nov. 1, 2018, which is a continuation of, and claims priority to, U.S. patent application Ser. No. 13/508,466, filed Nov. 13, 2012 (abandoned), which is the U.S. national phase application filed under 35 U.S.C. § 371 claiming benefit to International Patent Application No. PCT/US2010/056064, filed on Nov. 9, 2010 which is entitled to priority under 35 U.S.C. § 119(e) to U.S. Provisional Patent Application No. 61/259,435 filed on Nov. 9, 2009, all of which application are hereby incorporated herein by reference their entirety.

### BACKGROUND OF THE INVENTION

Injuries to soft tissue, for example, vascular, skin, or musculoskeletal tissue, are quite common. Soft tissue conditions further include, for example, conditions of skin (e.g., scar revision or the treatment of traumatic wounds, severe burns, skin ulcers (e.g., decubitus (pressure) ulcers, venous ulcers, and diabetic ulcers), and surgical wounds such as those associated with the excision of skin cancers); vascular condition (e.g., vascular disease such as peripheral arterial disease, abdominal aortic aneurysm, carotid disease, and venous disease; vascular injury; improper vascular development); conditions affecting vocal cords; cosmetic conditions (e.g., those involving repair, augmentation, or beautification); muscle diseases (e.g., congenital myopathies; myasthenia gravis; inflammatory, neurogenic, and myogenic muscle diseases; and muscular dystrophies such as Duchenne muscular dystrophy, Becker muscular dystrophy, myotonic dystrophy, limb-girdle-muscular dystrophy, facioscapulohumeral muscular dystrophy, congenital muscular dystrophies, oculopharyngeal muscular dystrophy, distal muscular dystrophy, and Emery-Dreifuss muscular dystrophy); conditions of connective tissues such as tendons and ligaments, including but not limited to a periodontal ligament and anterior cruciate ligament; and conditions of organs and/or fascia (e.g., the bladder, intestine, pelvic floor).

Surgical approaches to correct soft tissue defects in the body generally involve the implantation of structures made of biocompatible, inert materials that attempt to replace or substitute for the defective function. Implantation of non-biodegradable materials results in permanent structures that remain in the body as a foreign object. Implants that are made of resorbable materials are suggested for use as temporary replacements where the object is to allow the healing process to replace the resorbed material. However, these approaches have met with limited success for the long-term correction of structures in the body.

Degenerated and damaged soft tissues of the musculoskeletal system cause and increase the risk of medical complications resulting in intense pain and restricted motion. For example, degenerated and damaged soft tissues of the spine represent the major source of back pain for millions of people around the world. Soft tissue degeneration of the ligaments and intervertebral discs also increase the risk of damage to and back pain from local spinal joints, including: zygapophysical (facet), costovertebral, sacroiliac, sacral vertebral and atlantoaxial joints.

There generally are two types of bone conditions in humans: 1) non-metabolic bone conditions, such as bone fractures, bone/spinal deformation, osteosarcoma, myeloma, bone dysplasia and scoliosis, and 2) metabolic bone conditions, such as osteoporosis, osteomalacia, rickets, fibrous osteitis, renal bone dystrophy and Paget's disease of bone. Osteoporosis, a metabolic bone condition, is a systemic disease characterized by increased bone fragility and fracturability due to decreased bone mass and change in fine bone tissue structure. The major clinical symptoms of osteoporosis includes spinal kyphosis, and fractures of dorsolumbar bones, vertebral centra, femoral necks, lower end of radius, ribs, upper end of humerus, and others. In bone tissue, bone formation and destruction due to bone resorption occur constantly. Upon deterioration of the balance between bone formation and bone destruction due to bone resorption, a quantitative reduction in bone occurs. Traditionally, bone resorption suppressors such as estrogens, calcitonin and bisphosphonates have been mainly used to treat osteoporosis.

With respect to bone/spinal conditions, over 75% of the American population suffers from back pain sometime during their life. Underlying medical illnesses can contribute to back pain. These include scoliosis, spinal stenosis, degenerative disc disease, infectious processes, tumors, and trauma. The repair of large segmental defects in diaphyseal bone is a significant problem faced by orthopaedic surgeons today. Although such bone loss may occur as the result of acute injury, these massive defects commonly present secondary to congenital malformations, benign and malignant tumors, osseous infection, and fracture non-union. The use of fresh autologous bone graft material has been viewed as the historical standard of treatment but is associated with substantial morbidity including infection, malformation, pain, and loss of function (Kahn et al., 1995, Clin. Orthop. Rel. Res. 313:69-75). The complications resulting from graft harvest, combined with its limited supply, have inspired the development of alternative strategies for the repair of clinically significant bone defects. The primary approach to this problem has focused on the development of effective bone implant materials.

Three general classes of bone implants have emerged from these investigational efforts, and these classes may be categorized as osteoconductive, osteoinductive, or directly osteogenic. Allograft bone is probably the best known type of osteoconductive implant. Although widely used for many years, the risk of disease transmission, host rejection, and lack of osteoinduction compromise its desirability (Leads, 1988, JAMA 260:2487-2488). Synthetic osteoconductive implants include titanium fibermetals and ceramics composed of hydroxyapatite and/or tricalcium phosphate. The favorably porous nature of these implants facilitate bony ingrowth, but their lack of osteoinductive potential limits their utility. A variety of osteoinductive compounds have also been studied, including demineralized bone matrix, which is known to contain bone morphogenic proteins (BMP). Since the original discovery of BMPs, others have characterized, cloned, expressed, and implanted purified or recombinant BMPs in orthotopic sites for the repair of large bone defects (Gerhart et al., 1993, Clin. Orthop. Rel. Res. 293:317-326; Stevenson et al., 1994, J. Bone Joint Surg. 76:1676-1687; Wozney et al., 1988 Science 242:1528-1534). The success of this approach has hinged on the presence of mesenchymal cells capable of responding to the inductive signal provided by the BMP. It is these mesenchymal progenitors which undergo osteogenic differentiation and are ultimately responsible for synthesizing new bone at the surgical site.

One alternative to the osteoinductive approach is the implantation of living cells which are directly osteogenic. Since bone marrow has been shown to contain a population of cells which possess osteogenic potential, some have devised experimental therapies based on the implantation of fresh autologous or syngeneic marrow at sites in need of skeletal repair (Grundel et al., 1991, Clin. Orthop. Rel. Res. 266:244-258; Werntz et al., 1996, J. Orthop. Res. 14:85-93; Wolff et al., 1994, J. Orthop. Res. 12:439-446). Though sound in principle, the practicality of obtaining enough bone marrow with the requisite number of osteoprogenitor cells is limiting.

The leading cause of back pain is due to degeneration of the intervertebral disc. This degeneration leads to additional changes in the spine as the disc degenerates and loses height. The disc is composed of the annulus, the nucleus and end plates. The interface between vertebral bone and the soft tissue of the inter-vertebral disc is referred to as the endplate. The bone of the vertebral endplates are contiguous with vertebrae and they are covered with a cartilaginous surface, therefore, the endplate is a cartilage layer along with sub-chondral vertebral bone. The disc soft tissues between the endplates are the annulus fibrosis and nucleolus pulposus. The annulus fibrosis is a fibrous tissue that surrounds and contains the nucleus pulposus.

The nucleus pulposus is a matrix of various components, including nucleus pulpopus cells, collagen, elastin and proteoglycans such as aggrecan. Aggrecan is an extremely large molecule (2-5×10⁶ Da) composed of a protein core, condroitin sulfate and keratan sulfate along with linker proteins and oligosaccharides and can assemble extracellularly with hyaluronic acid (HA) to form an aggregated aggrecan molecule nucleus pulposus cells express each of the components of aggrecan, and assemble the molecules intracellularly For the aggregated aggrecan, HA is the backbone where the other components attach to the backbone. It is known that the number and activity of the nucleus pulposus cells drop over time. The aggrecan in the disc nucleus pulposus provides the disc with an osmotic pressure, which draws water into the nucleus increasing pressure within the disc. This tensions the annulus and so the intervertebral disc carries a great deal of the load imparted to the spine. The pressures in the disc space range from 0.1 MPa while laying supine to 0.8 MPa while walking to over 1 MPa while lifting a load. This osmotic pressure allows the disc to shed or imbibe water during the course of a normal day. For instance, it is well known that the disc loses water volume and height during the day and regains the height as a person rests, lying prone. This causes water and nutrients to flow in and out of the disc daily by convection.

Aggrecan and other similar proteoglycans comprise 15% wet weight of the inner region (nucleus pulposus) of the intervertebral disc (Prithvi et al., 2008 Pain Practice 8: 18-44). Aggrecan works to resist mechanical force in the nucleus pulposus and provide a hydrostatic tension to the outer region of the intervertebral disc via molecular interactions. Aggrecan is composed of a protein core to which glycosaminoglycans (GAGs) such as chondroitin sulfate (CS) and keratan sulfate (KS) are covalently bound. CS consists of repeating disaccharide units of N-acetylgalactosamine (GalN) and glucuronic acid (GlcN). Charged anionic groups on the GAG chains draw water into the disc and electrostatic repulsions generated between closely packed GAG chains resist deformation thereby allowing the tissue to distribute mechanical forces. Theoretical modeling has predicted that electrostatic repulsion forces account for up to 50% of the equilibrium compressive elastic modulus of cartilage, but these forces will only occur when intermolecular distances are 2-4 nm or less (Seog et al., 2002 Macromolecules 35: 5601-5615).

It is also know that the aggrecan molecular weight and concentration decreases as the disc ages. This reduces the water imbibing characteristics of the disc or osmotic potential as well as the electrostatic repulsion forces. As the osmotic potential of the nucleus material reduces the amount of water stored by the nucleus material drops, thereby reducing the volume of nucleus material and the internal pressure. This reduces the ability of the disc to share load, which in turn causes the annulus to carry more load. This causes the annulus to degenerate. The reduction in pressure in the disc also causes the motion at the disc to be more lax. This successive degeneration is often referred to as the degenerative cascade.

While the mainstay of treatment for degenerated inter-vertebral disc is fusion, a number of treatment methods and materials for repairing or replacing intervertebral discs have been proposed. Two developmental approaches exist to surgically repair or replace intervertebral discs: the first one focuses on designing artificial total discs, the other targets artificial nucleus.

The artificial total disc is developed to replace the complete disc structures: annulus fibrosus, nucleus pulposus and endplates. Artificial discs are challenged by both biological and biomechanical considerations, and often require complex prosthesis designs.

Nucleus replacement, which includes components of aggregated aggrecan (e.g., protein core, condroitin sulfate, keratan sulfate and HA), is an advantage over using artificial total disc. One advantage of nucleus replacement is the preservation of disc tissues (i.e., the annulus and the endplates). Nucleus replacement also allows for the maintenance of the biological functions of the natural tissues. Furthermore the replacement of the nucleus is surgically less complicated and less risky than undergoing a total intervertebral disc replacement. One limitation of the nucleus replacement procedure resides in the need of relatively intact annulus and endplates, which means the nucleus replacement procedure must be performed when disc degeneration is at an early stage.

The use of soft tissue implants for cosmetic applications (aesthetic and reconstructive) is common in breast augmentation, breast reconstruction after cancer surgery, craniofacial procedures, reconstruction after trauma, congenital craniofacial reconstruction and oculoplastic surgical procedures to name a few. The clinical function of a soft tissue implant depends upon the implant being able to effectively maintain its shape over time. In many instances, for example, when these devices are implanted in the body, they are subject to a “foreign body” response from the surrounding host tissues. The body recognizes the implanted device as foreign, which triggers an inflammatory response followed by encapsulation of the implant with fibrous connective tissue. Encapsulation of surgical implants complicates a variety of reconstructive and cosmetic surgeries, and is particularly problematic in the case of breast reconstruction surgery where the breast implant becomes encapsulated by a fibrous connective tissue capsule that alters the anatomy and function. Scar capsules that harden and contract (known as “capsular contractures”) are the most common complication of breast implant or reconstructive surgery. Capsular (fibrous) contractures can result in hardening of the breast, loss of the normal anatomy and contour of the breast, discomfort, weakening and rupture of the implant shell, asymmetry, infection, and patient dissatisfaction. Further, fibrous encapsulation of any soft tissue implant can occur even after a successful implantation if the device is manipulated or irritated by the daily activities of the patient.

Scarring and fibrous encapsulation can also result from a variety of other factors associated with implantation of a soft tissue implant. For example, unwanted scarring can result from surgical trauma to the anatomical structures and tissue surrounding the implant during the implantation of the device. Bleeding in and around the implant can also trigger a biological cascade that ultimately leads to excess scar tissue formation. Similarly, if the implant initiates a foreign body response, the surrounding tissue can be inadvertently damaged from the resulting inflammation, leading to loss of function, tissue damage and/or tissue necrosis. Furthermore, certain types of implantable prostheses (such as breast implants) include gel fillers (e.g., silicone) that tend to leak through the membrane envelope of the implant and can potentially cause a chronic inflammatory response in the surrounding tissue (which augments tissue encapsulation and contracture formation). When scarring occurs around the implanted device, the characteristics of the implant-tissue interface degrade, the subcutaneous tissue can harden and contract and the device can become disfigured. The effects of unwanted scarring in the vicinity of the implant are the leading cause of additional surgeries to correct defects, break down scar tissue, or remove the implant.

There is a need in the art to provide a novel minimally-invasive method for restoring damaged or degenerated soft tissue, including intervertebral discs. For example, a novel minimally-invasive method for obtaining restoration soft tissue functions at an early stage is desirable. Moreover, a novel minimally-invasive method for obtaining restoration of disc functions at an early stage, particularly before any advanced degeneration or damages resulting into disc rupture and fragmentation is desirable. The present invention satisfies this need.

### BRIEF SUMMARY OF THE INVENTION

The present invention provides a composition comprising a biomimetic proteoglycan. The biomimetic proteoglycan comprises a glycosaminoglycan (GAG) that is attached to a core structure.

In one embodiment, the GAG is selected from the group consisting of hyaluronic acid, chondroitin, chondroitin sulfate, heparin, heparin sulfate, dermatin, dermatin sulfate, laminin, keratan sulfate, chitin, chitosan, acetyl-glucosamine, oligosaccharides, and any combination thereof.

In one embodiment, the core structure is selected from the group consisting of a synthetic polymer, a protein, a peptide, a nucleic acid, a carbohydrate and any combination thereof.

In one embodiment, the core structure is a synthetic polymer selected from the group consisting poly(4-vinylphenyl boronic acid), poly (3,3′-diethoxypropyl methacylate), polyacrolein, poly(N-isopropyl acrylamide-co-glycidyl methacrylate), poly(allyl glycidyl ether), poly(ethylene glycol), poly(acrylic acid), and any combination thereof.

In one embodiment, the synthetic polymer renders the biomimetic proteoglycan resistant to enzymatic breakdown in a mammalian in vivo environment.

In one embodiment, the GAG comprises a terminal handle selected from the group consisting of a terminal primary amine, terminal diol, and an introduced aldehyde.

In one embodiment, the GAG is attached to the core structure by way of a linking chemistry selected from the group consisting of a bornic acid-diol linkage, epoxide-amine linkage, aldehyde-amine linkage, carboxylic acid-amine linkage, sulfhydryl-maleimide linkage, and any combination thereof.

In one embodiment, the biomimetic proteoglycan has a shape selected from the group consisting of cyclic, linear, branched, star-shaped, comb, graft, bottlebrush, dendritic, mushroom, and any combination thereof.

In one embodiment, the biomimetic proteoglycan mimics natural proteoglycan selected from the group consisting of aggrecan, betaglycan, decorin, perlecan, serglycin, syndecan-1, biglycan, fibromodulin, lumican, versican, neurocan, brevican, and any combination thereof.

In one embodiment, the biomimetic proteoglycan is biomimetic aggrecan and the GAG is selected from the group consisting of chondroitin sulfate, keratin sulfate, oligosaccharides, and combination thereof.

The invention provides a method of generating a biomimetic proteoglycan. The method comprises attaching a glycosaminoglycan (GAG) to a core structure.

In one embodiment, the GAG is selected from the group consisting of hyaluronic acid, chondroitin, chondroitin sulfate, heparin, heparin sulfate, dermatin, dermatin sulfate, laminin, keratan sulfate, chitin, chitosan, acetyl-glucosamine, oligosaccharides, and any combination thereof.

In one embodiment, the core structure is selected from the group consisting of a synthetic polymer, a protein, a peptide, a nucleic acid, a carbohydrate, and any combination thereof.

In one embodiment, the core structure is a synthetic polymer selected from the group consisting poly(4-vinylphenyl boronic acid), poly (3,3′-diethoxypropyl methacylate), polyacrolein, poly(N-isopropyl acrylamide-co-glycidyl methacrylate), poly(allyl glycidyl ether), poly(ethylene glycol), poly(acrylic acid), and any combination thereof.

In one embodiment, the synthetic polymer renders said biomimetic proteoglycan resistant to enzymatic breakdown in a mammalian in vivo environment.

In one embodiment, the GAG comprises a terminal handle selected from the group consisting of a terminal primary amine, terminal diol, and an introduced aldehyde.

In one embodiment, the GAG is attached to the core structure by way of a linking chemistry selected from the group consisting of a bornic acid-diol linkage, epoxide-amine linkage, aldehyde-amine linkage, carboxylic acid-amine linkage, sulfhydryl-maleimide linkage, and any combination thereof.

In one embodiment, the biomimetic proteoglycan has a shape selected from the group consisting of cyclic, linear, branched, star-shaped, comb, graft, bottlebrush, dendritic, mushroom, and any combination thereof.

In one embodiment, the biomimetic proteoglycan mimics natural proteoglycan selected from the group consisting of aggrecan, betaglycan, decorin, perlecan, serglycin, syndecan-1, biglycan, fibromodulin, lumican, versican, neurocan, brevican, and any combination thereof.

In one embodiment, the biomimetic proteoglycan is biomimetic aggrecan and the GAG is selected from the group consisting of chondroitin sulfate, keratin sulfate, oligosaccharides, and combination thereof.

The method provides a method of treating a disease, disorder, or condition associated with a soft tissue in a mammal. The method comprises administering a composition comprising a biomimetic proteoglycan to a mammal in need thereof. Preferably, the mammal is a human.

In one embodiment, the biomimetic proteoglycan is capable of water uptake and is further electrostatically active in said mammal.

In one embodiment, the said soft tissue is selected from the group consisting of intervertebral disc, skin, heart valve, articular cartilage, cartilage, meniscus, fatty tissue, craniofacial, ocular, tendon, ligament, fascia, fibrous tissue, synovial membrane, muscle, nerves, blood vessel, and any combination thereof.

In one embodiment, the biomimetic proteoglycan mimics proteoglycan selected from the group consisting of aggrecan, betaglycan, decorin, perlecan, serglycin, syndecan-1, biglycan, fibromodulin, lumican, versican, neurocan, brevican, and any combination thereof.

In one embodiment, the biomimetic proteoglycan is a biomimetic aggrecan.

In one embodiment, the composition comprising a biomimetic proteoglycan further comprises a cell. In some instances, the cell is genetically modified.

In one embodiment, the composition comprising a biomimetic proteoglycan further comprises at least one biologically active molecule. Preferably, the biologically active molecule is a growth factor, cytokine, antibiotic, protein, anti-inflammatory agent, or analgesic.

In one embodiment, the composition comprising a biomimetic proteoglycan further comprises a biocompatible matrix. In some instances, the biocompatible matrix is selected from the group consisting of calcium alginate, agarose, fibrin, collagen, laminin, fibronectin, glycosaminoglycan, hyaluronic acid, heparin sulfate, chondroitin sulfate A, dermatan sulfate, bone matrix gelatin, and any combination thereof. In some instances, the biocompatible matrix comprises a synthetic component.

In one embodiment, the composition comprising a biomimetic proteoglycan further comprises a non-solvent carrier. In some instances, the composition comprising a biomimetic proteoglycan further comprises a solvent carrier. In some instances, the composition comprising a biomimetic proteoglycan is dried.

In one embodiment, the disease, disorder, or condition is a degenerated disc and the composition is administered to the mammal by an approach selected from the group consisting of a posterior approach, a posterolateral approach, an anterior approach, an anterolateral approach, and a lateral approach.

In one embodiment, the composition is administered through endplates.

In one embodiment, the disease, disorder, or condition is a degenerated skin and the composition is administered to the mammal by an approach selected from the group consisting of intradermal, injection, subdermal injection, subcutaneous injection, diffusion, and implantation.

In one embodiment, the disease, disorder, or condition is osteoarthritis and the composition is administered to the mammal by an approach to the diarthrodial joints selected from group consisting of injection, arthroscopic implantation, and open implantation.

The invention provides a kit comprising a biomimetic proteoglycan, an applicator, and a delivery device. In one embodiment, the kit further comprises an instruction manual.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is an image depicting Thompson scale of grading degenerated intervertebral discs.

FIG. 2 is an image depicting revolved axisymmetric model of anterior column unit.

FIG. 3, comprising FIGS. 3A and 3B, is a series of images depicting fixed charge density profiles for A) a 26 year old healthy disc and a 74 year old degenerated disc, and B) the interpolated fixed charge density profiles for all grades.

FIG. 4 is an image depicting initial fixed charge density profiles for grade 1 (top) through grade 5 (bottom).

FIG. 5, comprising FIGS. 5A and 5B, is a series of images depicting total fluid loss (%) for A) all cycles and B) steady-state cycle.

FIG. 6, comprising FIGS. 6A and 6B, is a series of images depicting von Mises stress contour plots at end of loading cycle for grades 1 through 5 of A) nucleus pulposus and annulus fibrosus and B) nucleus pulposus only for grade 1 (top) through grade 5 (bottom).

FIG. 7 is an image depicting osmotic pressure gradient at end of loading cycle for grade 1 (top) through grade 5 (bottom).

FIG. 8 is an image demonstrating a comparison of the stress profiles for the unaltered nucleus pulposus (left column) and after the restoration of the chondroitin sulfate profile (right column).

FIG. 9 is an image demonstrating a comparison of the stress profiles for the unaltered annulus fibrosus (left column) and after the restoration of the chondroitin sulfate profile (right column).

FIG. 10 is an image demonstrating the pressure change in the NP with increasing implanted hydrogel volume.

FIG. 11 is an image depicting the stiffness of the augmented ACU is increased over the intact in tension (p<0.02) and through zero loading (p<0.02), but not at higher loading levels.

FIG. 12 is an image demonstrating that aggrecan is a bottle brush molecule with a protein core and chondroitin and keratan sulfate bristles. (Roughley P J et al. European Spine Journal. 2006; 15:326-32 and Ng L et al. Journal of Structural Biology. 2003; 143(3):242-57).

FIG. 13 is a schematic of enzymatic degradation of aggrecan where enzymatic cleavage is targeted to the core protein (Kiani C et al. 2002; 12(1):19-32.)

FIG. 14, comprising FIGS. 14A and 14B, is a series of images depicting a schematic of strategy for biomimetic aggrecan and pathways to the fabrication of biomimetic aggrecan with resulting examples synthetic polymeric backbones, respectively.

FIG. 15 is an image depicting the strategy for the synthesis of biomimetic aggrecan via the interaction of a CS terminal diol with a boronic acid polymer.

FIG. 16 is an image depicting CS with depicted repeat disaccharide, oligosaccharide linkage region and amino acid residue from cleavage at serine from the protein backbone. Cleavage leaves a primary amine attached at the terminal end of CS.

FIG. 17 is an image demonstrating that primary amine terminated CS was conjugated amine reactive monomers at varying monomer:CS ratio. Conjugation was detected using the fluorescamine assay.

FIG. 18, comprising FIGS. 18A and 18B, is a series of image depicting ¹H-NMR spectra of (a) CS and (b) CS-AGE solutions in D₂O. Peaks corresponding to residues in the structure of AGE, as well as the CS disaccharide are identified. Integrated area is indicated for peaks 6, 5, and 4 in (b).

FIG. 19 is an image depicting ¹H-NMR spectra of CS-AGE conjugate reactions over a 96 hr period.

FIG. 20 is an image depicting contact angle measurements on glass surfaces functionalized with CS via the terminal primary amine (significance determined by 2-way ANOVA with post-hoc analysis, p<0.05 considered significant, n>5).

FIG. 21 is an image depicting synthesis strategy for the fabrication of biomimetic aggrecan utilizing the CS terminal primary amine and the “grafting-to” strategy of synthesis.

FIG. 22 is an image depicting schematic representation of the “grafting-to” technique of polymerization utilizing a PAA backbone and CS bristles.

FIG. 23 is an image depicting % Conjugation of CS to PAA over time with varying ionic concentration, temperature and CS:PAA molar ratio.

FIG. 24 is an image depicting viscosity of PAA based biomimetic aggrecan in comparison to aggrecan, CS, and a simple mix of CS and PAA. Sample concentration was lmg/mL in PBS and studies were conducted at 25° C.

FIG. 25 is an image depicting dried CS-PAA conjugate labeled with hydrazide dye Alexa fluor 488 fluorescent label.

FIG. 26 is an image depicting ¹H-NMR of CS-AGE conjugate (monomer) and CS-AGE after free radical polymerization with APS/TMEDA (AGE-based biomimetic aggrecan).

FIG. 27 is an image depicting schematic representation of the synthesis of PEG and EG based biomimetic aggrecan.

FIG. 28 is an image depicting reaction kinetics at varying temperatures for the reaction of CS to G-DGE, EG-DGE, and PEG-DGE as monitored by the fluorescamine assay.

FIG. 29 is an image depicting reaction kinetics for the reaction of CS to EG-DGE and PEG-DGE di-epoxides as monitored by the fluorescamine assay.

FIG. 30 is an image depicting ¹NMR spectra for PEG and EG based biomimetic aggrecan before and after purification.

FIG. 31 is an image depicting TEM images of CS, natural aggrecan, and PEG-DGE-CS brushes after 24 and 72 hrs of reaction.

FIG. 32 is an image depicting specific viscosity of PEG and EG based biomimetic aggrecan.

FIG. 33 is an image depicting NIH 3T3 Fibroblast cultures dosed with di-epoxide monomer and PEG/EG based biomimetic aggrecan and cultured for 48 hrs. Cultures were stained with calcein AM for live cell cytoplasm (green) and ethidium homodimer-1 for dead cell nuclei (red).

FIG. 34 is an image depicting periodate oxidation of CS to introduce an aldehyde handle for biomimtic aggrecan synthesis (Dawlee S et al. Biomacromolecules. 2005; 6(4):2040-8.)

### DETAILED DESCRIPTION

The present invention is based partly on the discovery that a hybrid synthetic/bio-based macromolecular bottle brush structure can be synthesized to incorporate chondroitin sulfate. An additional innovation comes from the enzymatically resistant molecular design that can advance the survival of the molecule in vivo, while maintaining molecular function. The approach is significant because it facilitates an understanding of processing strategies and resulting structures and their property relations, thus enabling a family of tunable biomacromolecules for use in various applications of soft-tissue restoration.

The invention relates to the use of a number of different strategies to generate a biomimetic proteoglycan, such as aggrecan. Different handles on the chondroitin sulfate may be utilized including a terminal diol, a terminal primary amine or an introduced aldehyde group. These handles can be covalently bound to a synthetic component via several different linking chemistries including boronic acid, aldehyde, epoxide, carboxylic acid and sulfhydryl interactions. The biomimetic aggrecan can be polymerized into a bottle brush structure via the “grafting-to” or “grafting-through” polymerization strategies. The resulting structure exhibits characteristics of natural chondroitin sulfate bristles.

The present invention encompasses methods and compositions for treating diseases, disorders, or conditions associated with soft tissue defects and disorders, where administration of a proteoglycan to the soft tissue site results in functional restoration of the soft tissue, in whole or in part. In one example, the invention includes compositions and methods for treating a degenerated disc.

For the purposes of the present invention, a soft tissue defect or disorder includes but is not limited to degeneration or damage to skin, heart valves, articular cartilage, cartilage, meniscus, fatty tissue, craniofacial, ocular, disc, and the like. The invention is also useful for repair, restoration or augmentation of soft tissue defects or contour abnormalities. Thus, while the invention is described using as examples, repair of degenerated discs, the invention should be read at all times to include repair of defects in any soft tissue in the body, as the term soft tissue is defined herein. While the precise compositions used and the methods of administration of the materials of the invention may vary from tissue to tissue, the skilled artisan will know, based on the disclosure provided herein, how to adapt the disclosure relating to disc repair to repair of other soft tissue, to the extent that such adaption has not been disclosed in detail herein.

In one embodiment, the present invention relates to the development of a biomimetic replacement for a ubiquitous biomacromolecule (e.g., proteoglycan) for use as a minimally invasive early interventional technique for the treatment and prevention of back pain originating from intervertebral disc degeneration. Proteoglycans are molecules that contain both a protein portion (which may be referred to as the protein core) and glycosaminoglycan portion. Glycosaminoglycans are the most widely present polysaccharides in the animal kingdom and are mainly found in the connective tissues. Glycosaminoglycans are biological polymers made up of linear disaccharide units containing an uronic acid and a hexosamine and are attached to the core proteins via a linking tetrasaccharide moiety. The major glycosaminoglycans are hyaluronic acid, chondroitin sulfates, heparan sulfate, dermatan sulfate and keratan sulfate.

In one embodiment, the biomimetic replacement is biomimetic aggrecan. However, the invention should not be construed to be limited to aggrecan, but should be construed to include other types of biomimetic proteoglycan, including but not limited to, betaglycan, decorin, perlecan, serglycin, syndecan-1, biglycan, fibromodulin, lumican, and the like. The invention also includes the hyalectan (lectican) family of proteoglycans which bind to hyaluronan including but not limited to versican, aggrecan, neurocan, brevican, and the like.

A proteoglycan has two main mechanical functions: 1) it allows water uptake due to sulfated groups in the glycosaminoglycans and 2) it provides electrostatic repulsion due to the three-dimensional macromolecular structure. In one embodiment, biomimetic proteoglycan is based on the three-dimensional brush-like structure of a representative proteoglycan.

The invention relates to the use of a number of different strategies to generate a biomimetic proteoglycan, such as aggrecan. Different handles on the chondroitin sulfate may be utilized including a terminal diol, a terminal primary amine or an introduced aldehyde group. These handles can be covalently bound to a synthetic component via several different linking chemistries including boronic acid, aldehyde, epoxide, carboxylic acid and sulfhydryl interactions. The biomimetic aggrecan can be polymerized into a bottle brush structure via the “grafting-to” or “grafting-through” polymerization strategies. The resulting structure exhibits characteristics of natural chondroitin sulfate bristles.

In one embodiment, the biomimetic proteoglycan is generated by attaching a glycosaminoglycan to a polymer or otherwise a polymer backbone which serves as the protein portion (which may be referred to as the protein core) of the biomimetic proteoglycan. For example, the biomimetic aggrecan can be formed by the attachment of a terminal diol in chondroitin sulfate to a boronic acid polymer. Utilizing the high affinity complexation of boronic acids with compounds containing diols (such as saccharides), a novel polymer system has been developed to generate biomimetic aggrecan. For example, a free radical polymerization technique which comprises using a boronic acid functionalized polymer core to attach chondroitin sulfate to form brush “bristles” to mimic the bristles of the aggrecan molecule. The applied engineering of the polymer structure using a biomimetic philosophy enables the development of an effective early stage treatment to the spine.

In another embodiment, the biomimetic proteoglycan of the invention can be generated by attaching a glycosaminoglycan through a terminal primary amine handle of the glycosaminoglycan to a polymer backbone. For example, biomimetic aggrecan can be generated by attaching chondroitin sulfate through a terminal primary amine handle to a polymer backbone. This technique is based on attaching a glycosaminoglycan to various monomers or polymers via a primary amine interaction that is likely only available in the terminal region of the glycosaminoglycan molecule. This allows for the controlled organization of glycosaminoglycan onto various polymeric backbones that may be tuned to match the properties desired for any therapy that is associated with treating a disease, disorder, or condition associated with dysfunctional proteoglycan. Preferably, the terminal primary amide strategy includes the use of a covalent linking chemistry including, but is not limited to aldehyde, epoxide, and carboxylic acid.

In another embodiment, the biomimetic proteoglycan of the invention can be generated using an epoxide strategy. For example, a CS terminal primary amine is reacted with a di-epoxide, where the primary amine of each CS chain is reactive with two epoxide moieties. The reaction of the CS terminal primary amine with the epoxides of the di-epoxide results in the generation of a biomimetic aggrecan polymer via linear step-growth polymerization. In some instances, this epoxide strategy is a type of “grafting-through” step-growth polymerization strategy.

In one embodiment, the biomimetic proteoglycan of the invention is a hydride synthetic/bio-based bottle brush structure. The biomimetic proteoglycan is an improvement over its corresponding natural counterpart at least because the biomimetic proteoglycan comprises an enzymatically resistant core. The enzymatic resistant property of the biomimetic proteoglycan is partly due to the synthetic polymer core replacing the protein core of natural aggrecan.

In one embodiment, the invention includes a method of administering a material (e.g., biomimetic aggrecan) into the nucleus of a degenerated disc in order to increase the osmotic potential of the disc. Administration of a material of the present invention into the nucleus of a degenerated disc can restore normal disc height and function. Such administration can result in whole or partial restoration of the load-bearing and viscoelastic properties of the defective intervertebral disc. The present invention can be used in conjunction with any known or heretofore unknown method of treating a disc disease or condition in a mammal. Preferably, the mammal is a human.

In one embodiment, the invention includes a kit comprising a biomimetic aggrecan, an introducer needle, and a delivery device for administering the biomimetic aggrecan. The biomimetic aggrecan may be administered as a solution or dry. In some instances, the kit further comprises an instruction manual.

The kit and method of making a kit can include the embodiments discussed herein with respect to the method of treating a disc as well as other embodiments disclosed herein.

Advantages of the biomimetic proteoglycan of the invention includes the ability of regulating enzymatic digestion of the biomimetic proteoglycan. The biomimetic proteoglycan may be made to resist or promote digestion in the polymer core of the biomimetic proteoglycan.

An additional advantage of the biomimetic proteoglycan of the invention is that it can be made large enough to resist migration out of the desired site of administration. For example, the biomimetic proteoglycan molecule can be made large enough to resist migration out of the nucleus pulposus/disc where chondroitin and keratan sulfate and other GAGs without a protein or polymer core migrate out of disc.

The biomimetic proteoglycan of the invention is advantageous because in the context of a disc, it can support and not interrupt natural disc circulation due to water migration in and out of the disc in response to natural disc loading and unloading. Therefore, the biomimetic proteoglycan can enhance and not interfere with cellular metabolic activity which is dependent on convection for the large molecule metabolites. Preferably, this property of the biomimetic proteoglycan is applicable in situations of nucleus augmentation without nucleus pulposus removal.

### Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

“Allogeneic” refers to a graft derived from a different animal of the same species.

As used herein, the term “autologous” is meant to refer to any material derived from the same individual to which it is later to be re-introduced into the individual.

“Xenogeneic” refers to a graft derived from a mammal of a different species.

As used herein, the term “biocompatible lattice,” is meant to refer to a substrate that can facilitate formation of three-dimensional structures conducive for tissue development. Thus, for example, cells can be cultured or seeded onto such a biocompatible lattice, such as one that includes extracellular matrix material, synthetic polymers, cytokines, growth factors, etc. The lattice can be molded into desired shapes for facilitating the development of tissue types. Also, at least at an early stage during culturing of the cells, the medium and/or substrate is supplemented with factors (e.g., growth factors, cytokines, extracellular matrix material, etc.) that facilitate the development of appropriate tissue types and structures.

“Bioactive agents,” as used herein, can include one or more of the following: chemotactic agents; therapeutic agents (e.g., antibiotics, steroidal and non-steroidal analgesics and anti-inflammatories (including certain amino acids such as glycine), anti-rejection agents such as immunosuppressants and anti-cancer drugs); various proteins (e.g., short term peptides, bone morphogenic proteins, collagen, hyaluronic acid, glycoproteins, and lipoprotein); cell attachment mediators; biologically active ligands; integrin binding sequence; ligands; various growth and/or differentiation agents and fragments thereof (e.g., epidermal growth factor (EGF), hepatocyte growth factor (HGF), vascular endothelial growth factors (VEGF), fibroblast growth factors (e.g., bFGF), platelet derived growth factors (PDGF), insulin derived growth factor (e.g., IGF-1, IGF-II) and transforming growth factors (e.g., TGFβ I-III), parathyroid hormone, parathyroid hormone related peptide, bone morphogenic proteins (e.g., BMP-2, BMP-4; BMP-6; BMP-7; BMP-12; BMP-13; BMP-14), sonic hedgehog, growth differentiation factors (e.g., GDF5, GDF6, GDF8), recombinant human growth factors (e.g., MP52, and MP-52 variant rhGDF-5), cartilage-derived morphogenic proteins (CDMP-1; CDMP-2, CDMP-3)); small molecules that affect the upregulation of specific growth factors; tenascin-C; hyaluronic acid; chondroitin sulfate; fibronectin; decorin; thromboelastin; thrombin-derived peptides; heparin-binding domains; heparin; heparan sulfate. Suitable effectors likewise include the agonists and antagonists of the agents described above. The growth factor can also include combinations of the growth factors described above. In addition, the growth factor can be autologous growth factor that is supplied by platelets in the blood. In this case, the growth factor from platelets will be an undefined cocktail of various growth factors. If other such substances have therapeutic value in the orthopedic field, it is anticipated that at least some of these substances will have use in the present invention, and such substances should be included in the meaning of “bioactive agent” and “bioactive agents” unless expressly limited otherwise. Preferred examples of bioactive agents include culture media, bone morphogenic proteins, growth factors, growth differentiation factors, recombinant human growth factors, cartilage-derived morphogenic proteins, hydrogels, polymers, antibiotics, anti-inflammatory medications, immunosuppressive mediations, autologous, allogenic or xenologous cells such as stem cells, chondrocytes, fibroblast and proteins such as collagen and hyaluronic acid. Bioactive agents can be autologus, allogenic, xenogenic or recombinant.

The term “biologically compatible carrier” or “biologically compatible medium” refers to reagents, cells, compounds, materials, compositions, and/or dosage formulations which are suitable for use in contact with the tissues of human beings and animals without excessive toxicity, irritation, allergic response, or other complication commensurate with a reasonable benefit/risk ratio.

As used herein, the term “bone condition (or injury or disease)” refers to disorders or diseases of the bone including, but not limited to, acute, chronic, metabolic and non-metabolic conditions of the bone. The term encompasses conditions caused by disease, trauma or failure of the tissue to develop normally. Examples of bone conditions include, but are not limited, a bone fracture, a bone/spinal deformation, osteosarcoma, myeloma, bone dysplasia, scoliosis, osteoporosis, osteomalacia, rickets, fibrous osteitis, renal bone dystrophy, and Paget's disease of bone.

“Differentiation medium” is used herein to refer to a cell growth medium comprising an additive or a lack of an additive such that a stem cell, adipose derived adult stromal cell or other such progenitor cell, that is not fully differentiated when incubated in the medium, develops into a cell with some or all of the characteristics of a differentiated cell.

“Functional restoration of a tissue” as that phrase is used herein, refers to the restoration of at least one function to a tissue, which function has been lost by the tissue as a result of a disorder or defect.

The terms “glycosaminoglycan” and “GAG”, as used interchangeably herein, refer to a macromolecule comprised of carbohydrate. The GAGs for use in the present invention may vary in size and be either sulfated or non-sulfated. The GAGs which may be used in the methods of the invention include, but are not limited to, hyaluronic acid, chondroitin, chondroitin sulfates (e.g., chondroitin 6-sulfate and chondroitin 4-sulfate), heparin, heparin sulfate, dermatin, dermatin sulfate, laminin, keratan sulfate, chitin, chitosan, acetyl-glucosamine, and the like.

By “growth factors” is intended the following specific factors including, but not limited to, growth hormone, erythropoietin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor (EGF), fibroblast growth factor (FGF), nerve growth factor, ciliary neurotrophic factor, platelet derived growth factor (PDGF), and bone morphogenetic protein at concentrations of between picogram/ml to milligram/ml levels.

As used herein, the term “growth medium” is meant to refer to a culture medium that promotes growth of cells. A growth medium will generally contain animal serum. In some instances, the growth medium may not contain animal serum.

An “isolated cell” refers to a cell which has been separated from other components and/or cells which naturally accompany the isolated cell in a tissue or mammal.

“Metabolically absorbable” refers herein to any chemicals or materials that are a) safely accepted within the body with no adverse reactions, and b) completely eliminated from the body over time through natural pathways or internal consumption. “Metabolically acceptable” refers to any chemicals or materials that are safely accepted within the body with no adverse reactions or harmful effects.

As used herein, “soft tissue” refers to a tissue that connects, supports, or surrounds other structures and organs of the body. For example, soft tissue includes but is not limited to disc, collagen, meniscus, tendon, ligament, fascia, fibrous tissue, fat, synovial membrane, other connective tissue, muscle, nerves, blood vessel, and the like.

A “suitable intervertebral space” as the term is used herein means the space between adjacent vertebrae where a disc resides in a healthy spine but which is reduced in volume or partially devoid of disc material due to wear and tear or has been prepared using techniques known in the art to surgically create a void in the disc space

As used herein, a “therapeutically effective amount” is the amount of material sufficient to provide a beneficial effect to the subject to which the material is administered.

“Treating (or treatment of)” refers to ameliorating the effects of, or delaying, halting or reversing the progress of, or delaying or preventing the onset of, a disease or degenerative condition.

As used herein “endogenous” refers to any material from or produced inside an organism, cell or system.

“Exogenous” refers to any material introduced into or produced outside an organism, cell, or system.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The phrase “under transcriptional control” or “operatively linked” as used herein means that the promoter is in the correct location and orientation in relation to the polynucleotides to control RNA polymerase initiation and expression of the polynucleotides.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (i.e., naked or contained in liposomes) and viruses that incorporate the recombinant polynucleotide.

As used herein, a “polymer backbone” refers to the moiety or structure for which GAGs, such as chondroitin sulfate, can attach to form a biomimetic proteoglycan. In some instances, the polymer backbone is considered the core structure, core portion, polymer core, or protein portion of the biomimetic proteoglycan, such as biomimetic aggrecan. In some instances, the polymer backbone can be a synthetic polymer, protein, peptide, nucleic acid, carbohydrate or combinations thereof.

As used herein, “mimics natural proteoglycan” means mimicking the structure and function of natural proteoglycan. “Mimics natural aggrecan” means mimicking the natural structure and function of natural aggrecan.

### DESCRIPTION

The present invention relates to the development of a biomimetic proteoglycan that is useful for treating diseases, disorders, defects or conditions associated with soft tissue. The biomimetic proteoglycan comprises both a core portion (which may be referred to as the polymer core or protein core) and a glycosaminoglycan portion. Any known glycosaminoglycan can be used to create the biomimetic proteoglycan by attaching the desired glycosaminoglycan to a polymer core. The glycosaminoglycan is assembled according to the methods of the invention into a bottle brush type of polymer as more fully explained elsewhere herein.

Without wishing to be bound by any particular theory, an advantage of the biomimetic proteoglycan of the invention includes the ability of regulating enzymatic digestion of the biomimetic proteoglycan. The biomimetic proteoglycan may be made to resist or promote digestion in the polymer core of the biomimetic proteoglycan. Another advantage of the biomimetic proteoglycan of the invention is that it can be made large enough to resist migration out of the desired site of administration. Yet another advantage is that in the context of a disc, the biomimetic proteoglycan can support and not interrupt natural disc circulation due to water migration in and out of the disc in response to natural disc loading and unloading. Therefore, the biomimetic proteoglycan can enhance and not interfere with cellular metabolic activity which is dependent on convection for the large molecule metabolites.

### Composition

The biomimetic proteoglycan of the invention comprises a glycosaminoglycan molecule attached to a core molecule. In one embodiment, the biomimetic proteoglycan functions similar to its natural proteoglycan that otherwise can be isolated from an animal or a cell, either by tissue extraction or by cell cultivation. For example, the biomimetic proteoglycan is spheroidal (e.g., bottle-brush-like spatial presentation or configuration) and functionally able to maintain high levels of hydration and exhibits sufficient mechanical properties.

The invention relates to the use of a number of different strategies to generate a biomimetic proteoglycan, such as aggrecan. Different handles on the GAG, such as chondroitin sulfate, may be utilized including a terminal diol, a terminal primary amine or an introduced aldehyde group. These handles can be covalently bound to a synthetic component via several different linking chemistries including boronic acid, aldehyde, epoxide, carboxylic acid and sulfhydryl interactions. The biomimetic aggrecan can be polymerized into a bottle brush structure via the “grafting-to” or “grafting-through” polymerization strategies. The resulting structure exhibits characteristics of natural proteoglycans with glycosaminoglycans bound to a core material.

In one embodiment, the biomimetic proteoglycan comprises a glycosaminoglycan (GAG) with a terminal handle that is attached with a linking chemistry to a core structure. Preferably, the linking chemistry is selected from the group consisting of a bornic acid-diol linkage, epoxide-amine linkage, aldehyde-amine linkage, carboxylic acid-amine linkage, sulfhydryl-maleimide linkage and any combination thereof.

Based on the disclosure herein, a skilled artisan would understand that the biomimetic proteoglycan of the invention can be engineered to encompass any type of glycosaminoglycan and combinations thereof with any type of core protein or polymer core. Accordingly, the invention includes the use of hyaluronic acid, chondroitin, chondroitin sulfates (e.g., chondroitin 6-sulfate and chondroitin 4-sulfate), heparin, heparan sulfate, dermatin, dermatan sulfate, laminin, keratan sulfate, chitin, chitosan, acetyl-glucosamine, and the like.

In one embodiment, the biomimetic proteoglycan can encompass any combination of glycosaminoglycans wherein each glycosaminoglycan can vary in length. Similarly, varying lengths of the polymer can be used in the construction of the biomimetic proteoglycan. Without wishing to be bound by any particular theory, glycosaminoglycan variations include but are not limited to varying length, sulfation pattern, molecular weight, chemical composition, and the like. These variations can affect the conformation, molecular weight, hydrating, mechanical and cell signaling functions of the biomimetic proteoglycan.

In another embodiment the glycosaminoglycan is grafted to a backbone polymer with a predetermined number of attachment sites. Accordingly, the density of glycosaminoglycan to polymer can be adjusted to correspond to the particular use of the biomimetic proteoglycan.

The biomimetic proteoglycan can also be designed to have a particular shape. For example, different types of polymeric backbones can be used to generate a biomimetic proteoglycan that may take on a number of configurations, which may be selected, for example, from cyclic, linear and branched configurations, among others. Branched configurations include star-shaped configurations (e.g., configurations in which three or more chains emanate from a single branch point), comb configurations (e.g., configurations having a main chain and a plurality of side chains, also referred to as “graft” or “bottlebrush” configurations), dendritic configurations (e.g., arborescent and hyperbranched polymers), mushroom side chains, and so forth. Thus, the biomimetic proteoglycan may have any shape, non-limiting examples of which include but is not limited to, cyclic, linear, branched, star-shaped, comb, graft, bottlebrush, dendritic, mushroom, and any combination thereof.

In another embodiment, any core backbone or polymer can be used for attachment of the desired glycosaminoglycan. Polymers which may be used as the core portion of the biomimetic proteoglycan include, but are not limited to, dextrans, styrene polymers, polyethylene and derivatives, polyanions including, but not limited to, polymers of heparin, polygalacturonic acid, mucin, nucleic acids and their analogs including those with modified ribose-phosphate backbones, polypeptides, polyglutamate, polyaspartate, carboxylic acid, phosphoric acid, and sulfonic acid derivatives of synthetic polymers; and polycations, including but not limited to, synthetic polycations based on acrylamide and 2-acrylamido-2-methylpropanetrimethylamine, poly(N-ethyl-4-vinylpyridine) or similar quarternized polypyridine, diethylaminoethyl polymers and dextran conjugates, polymyxin B sulfate, lipopolyamines, poly(allylamines), poly(dimethyldiallylammonium chloride), polyethyleneimine, polybrene, spermine, spermidine, protamine, the histone polypeptides, polylysine, polyarginine and polyornithine; and mixtures, derivatives and combinations of these are contemplated by the present invention. Linear and branched polymers may be used in the biomimetic proteoglycan of the present invention.

A variety of polymers from synthetic and/or natural sources can be used as the core protein portion of the biomimetic proteoglycan of the present invention. For example, lactic or polylactic acid or glycolic or polyglycolic acid can be utilized to form poly(lactide) (PLA) or poly(L-lactide) (PLLA) nanofibers or poly(glycolide) (PGA) nanofibers. The core can also be made from more than one monomer or subunit thus forming a co-polymer, terpolymer, etc. For example, lactic or polylactic acid and be combined with glycolic acid or polyglycolic acid to form the copolymer poly(lactide-co-glycolide) (PLGA). Other copolymers of use in the invention include poly(ethylene-co-vinyl) alcohol). In an exemplary embodiment, a core can comprise a polymer or subunit which is a member selected from an aliphatic polyester, a polyalkylene oxide, polydimethylsiloxane, polyvinylalcohol, polylysine, and combinations thereof. In another exemplary embodiment, a core can comprises two different polymers or subunits which are members selected from an aliphatic polyester, a polyalkylene oxide, polydimethylsiloxane, polyvinylalcohol, polylysine, and combinations thereof. In another exemplary embodiment, a core comprises three different polymers or subunits which are members selected from an aliphatic polyester, a polyalkylene oxide, polydimethylsiloxane, polyvinylalcohol, polylysine, and combinations thereof. In an exemplary embodiment, the aliphatic polyester is linear or branched. In another exemplary embodiment, the linear aliphatic polyester is a member selected from lactic acid (D- or L-), lactide, poly(lactic acid), poly(lactide) glycolic acid, poly(glycolic acid), poly(glycolide), glycolide, poly(lactide-co-glycolide), poly(lactic acid-co-glycolic acid), polycaprolactone and combinations thereof. In another exemplary embodiment, the aliphatic polyester is branched and comprises at least one member selected from lactic acid (D- or L-), lactide, poly(lactic acid), poly(lactide) glycolic acid, poly(glycolic acid), poly(glycolide), glycolide, poly(lactide-co-glycolide), poly(lactic acid-co-glycolic acid), polycaprolactone and combinations thereof which is conjugated to a linker or a biomolecule. In an exemplary embodiment, wherein said polyalkylene oxide is a member selected from polyethylene oxide, polyethylene glycol, polypropylene oxide, polypropylene glycol and combinations thereof.

As another example, the core protein portion of the biomimetic proteoglycan may be formed from functionalized polyester graft copolymers. The functionalized graft copolymers are copolymers of polyesters, such as poly(glycolic acid) or poly(lactic acid), and another polymer including functionalizable or ionizable groups, such as a poly(amino acid). In another embodiment, polyesters may be polymers of α-hydroxy acids such as lactic acid, glycolic acid, hydroxybutyric acid and valeric acid, or derivatives or combinations thereof. The inclusion of ionizable side chains, such as polylysine, in the polymer has been found to enable the formation of more highly porous particles, using techniques for making microparticles known in the art, such as solvent evaporation. Other ionizable groups, such as amino or carboxyl groups, may be incorporated, covalently or noncovalently, into the polymer to enhance porosity. For example, polyaniline could be incorporated into the polymer. These groups can be modified further to contain hydrophobic groups capable of binding load molecules.

In an exemplary embodiment, the core protein portion of the biomimetic proteoglycan can include one or more of the following: polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly(ethylene terephthalate), poly(vinyl acetate), polyvinyl chloride, polystyrene, polyvinyl pyrrolidone, pluronics, polyvinylphenol, saccharides (e.g., dextran, amylose, hyalouronic acid, poly(sialic acid), heparans, heparins, etc.); poly (amino acids), e.g., poly(aspartic acid) and poly(glutamic acid); nucleic acids and copolymers thereof.

In an exemplary embodiment, the core protein portion of the biomimetic proteoglycan can include one or more of the following: peptide, saccharide, poly(ether), poly(amine), poly(carboxylic acid), poly(alkylene glycol), such as poly(ethylene glycol) (“PEG”), poly(propylene glycol) (“PPG”), copolymers of ethylene glycol and propylene glycol and the like, poly(oxyethylated polyol), poly(olefinic alcohol), poly(vinylpyrrolidone), poly(hydroxypropylmethacrylamide), poly(α-hydroxy acid), poly(vinyl alcohol), polyphosphazene, polyoxazoline, poly(N-acryloylmorpholine), polysialic acid, polyglutamate, polyaspartate, polylysine, polyethyeleneimine, biodegradable polymers (e.g., polylactide, polyglyceride and copolymers thereof), polyacrylic acid.

### Primary Amine

The biomimetic proteoglycan can be produced by attaching the terminal primary amine of a glycosaminoglycan to a polymer core. The terminal primary amine strategy of the invention for generating a biomimetic proteoglycan is based on the use of a terminal primary amine in a glycosaminoglycan (e.g., chondroitin sulfate) to react with an amine reactive group on a polymer backbone to form a bottle brush macromolecule. As discussed elsewhere herein, any glycosaminoglycan and modification thereof can be attached to a polymer core of interest. Therefore, the biomimetic proteoglycan of the invention can be made to take on a number of configurations, such as cyclic, linear and branched configurations. Other configurations include star-shaped configurations (e.g., configurations in which three or more chains emanate from a single branch point), comb configurations (e.g., configurations having a main chain and a plurality of side chains, also referred to as “graft” or “bottlebrush” configurations), dendritic configurations (e.g., arborescent and hyperbranched polymers), mushroom side chains, and so forth.

Included in the amine strategy of the invention is exploiting amine reactive functionalities including but not limited to aldehyde-amine, epoxy-amine, and carboxylate-amine interactions. With respect to aldehyde-amine interactions, an aldehyde can be used to attach a polymerizable group to the CS primary amine which creates the schiffs base intermediate.

With respect to amine reactive polymers-carboxylate, carboxylates from poly(acrylic acid) can be modified with 1-ethyl-3-(3-dimethylaminopropyl)carbodiimide hydrochloride (EDC)/N-hydroxysulfosuccinimide (sulfo-NHS) to mediate formation of amide linkages between the carboxylates and amines. Any branched polymers with amines, sulfhydryles, histidine, and methionine side chains can be modified to contain carboxylic acids. The disclosed invention involves covalent coupling of chondroitin sulfate through its primary amine group to carboxyl groups on various polymeric materials via a carbodiimide-mediated reaction.

The chemical link between the core protein and the terminal primary amine of a glycosaminoglycan may comprise modified amino groups. A modified amino group is the amide linkage of a hydrophobic functional group comprising an alkyl acyl derived from fatty acids, or aromatic alkyl acyl derived from aromatic alkyl acids, which has a general formula [CxHyOz] where x is 2-36; y is 3-71; z is 1-4. It is preferable that z=1, which is the minimum required for amide bond with the amino group. The starting molecules however may have z greater than 1 prior to amide bond formation.

Another object of the present invention is to provide a method of attaching a hydrophobic group to the amino group of the proteoglycan. The modifications can be done by amide bond formation. As an example that is not intended to limit the scope of this invention, the carboxyl containing hydrophobic molecule can be attached to the amino group of the proteoglycan using a carbodiimide containing reagent such a 1-ethyl-3-(3-dimethylaminopropyl)-carbodiimide or dicyclohexylcarbodiimide. A carbodiimide reagent contains a functional group consisting of the formula RN═C═NR. During the process of coupling reaction, the activated carboxyl group (O-acylisourea-intermediate) can optionally be stabilized by forming N-hydroxysuccinimide ester using N-hydroxysuccinimide. This relatively stable intermediate can react with the amino group of to form for example amino-acyl bond or amide bond.

Another way to attach a hydrophobic group is to react the amino group with a fatty acid anhydride. For example, reaction of the amino groups with palmitic acid anhydride forms a long chain hydrophobic group comprising 16 carbons. Any fatty acid anhydride may be used in this fashion.

### Boronic Acid

Utilizing the high affinity complexation of boronic acids with compounds containing diols (such as saccharides), a novel polymer system has been developed to generate biomimetic aggrecan. For example, a free radical polymerization technique which consists of a boronic acid functionalized polymer core is used to attach chondroitin sulfate to form brush “bristles” to mimic the bristles of the aggrecan molecule. The applied engineering of the polymer structure using a biomimetic philosophy enables the development of an effective early stage treatment to the spine. However, the invention is not limited to biomimetic aggrecan and treatment of back pain. Rather, the invention includes the generation of generally a biomimetic proteoglycan and the treatment of any disease, disorder, or condition associated with defective or dysfunctional proteoglycan.

The terminal diol strategy of the invention that generates a biomimetic aggrecan is based on the use of a diol at the terminal end of a disaccharide (e.g., chondroitin sulfate) for attachment to a polymeric backbone via diol-boronic acid interactions. For example, in a chondroitin sulfate molecule, the terminal GluUA presents a diol unique to the end of the chondroitin sulfate molecule. The diol can subsequently bind with a boronic acid through the formation of an ester bond.

Polysaccharides that are useful in the present invention include glycosaminoglycans such as hyaluronic acid, chondroitin sulfate A, chondroitin sulfate C, dermatan sulfate, keratan sulfate, chitin, chitosan, heparin, and derivatives or mixtures thereof. Further, proteoglycans such as decorin, biglycan and fibromodulin may also be used in the present invention. Proteoglycans are components of the extracellular matrix of cartilage cells and contain one or more glycosaminoglycan molecules bound to a core protein. Furthermore, mixtures of various species of glycosaminoglycans or proteoglycans with various proteins, or mixtures of various species of glycosaminoglycans or proteoglycans with proteins can be used in the practice of the present invention.

An example of a useful boronic acid compound is phenylboronic acid and its derivatives that bind with high affinity to molecules containing vicinyl or closely opposed diols or carboxylic acids. However, the invention is not limited to phenylboronic acid, but includes any compound that contains a boronic acid group.

Polymers comprising phenylboronic acid moieties can be synthesized, for example, by reacting aminophenylboronic acid with acryloyl chloride (D. Shino et al., J. Biomater. Sci Polym. Ed., 7:697-701, 1996), followed by free-radical polymerization with acrylamide to produce poly(acrylamide-co-acrylamidophenylboronic acid).

The boronic acid containing polymers can have a number of other functionalities within the polymer chain, which can enhance such properties as water solubility, bioinertness, or charge. Additional polymeric components, domains, linking groups, and bioactive, prophylactic, or diagnostic materials can be added to the boroic acid containing polymer to modify its properties.

In one aspect of this example, boron-containing compounds are used to prepare the biomimetic proteoglycan of the invention. It is known that boronic acids form cyclic esters with saccharides and the reaction occurs reversibly and rapidly at ambient temperature. It has been demonstrated that boronic acids serve as a useful interface to selectively recognize saccharides in water.

Other examples of boronate moieties and compounds suitable for reversible binding of glucose are phenylboronic acid, 2-carboxyethaneboronic acid, 1,2-dicarboxyethaneboronic acid, β,β′-dicarboxyethaneboronate, β,γ-dicarboxypropaneboronate, 2-nitro- and 4-nitro-3-succinamidobenzene boronic acids, 3-nitro-4-(6-aminohexylamido)-phenyl boronic acid, {4-[(hexamethylenetetramine)methyl]phenyl}boronic acid, 4-(N-methyl)carboxamidobenzene boronic acid, 2-{[(4-boronphenyl)methyl]-ethylammonio}ethyl and compounds containing 2-{[(4-boronphenyl)methyl]diethylammonio}ethyl groups, succinyl-3-aminophenylboronic acid, 6-aminocaproyl-3-aminophenylboronic acid, 3-(N-succinimidoxycarbonyl) aminophenylboronate, p-(Ω-aminoethyl)phenylboronate, p-vinylbenzeneboronate, N-(3-dihydroxyborylphenyl)succinamic acid, N-(4-nitro-3-dihydroxyborylphenyl)succinamic acid, O-dimethylaminomethylbenzeneboronic acid, 4-carboxybenzeneboronic acid, 4-(N-octyl)carboxamidobenzeneboronic acid, 3-nitro-4-carboxybenzeneboronic acid, 2-nitro-4-carboxybenzeneboronic acid, 4-bromophenylboronate, p-vinylbenzene boronate, 4-(Ω-aminoethyl)phenylboronate, catechol[2-(diethylamino)carbonyl, 4-bromomethyl]phenyl boronate, and 5-vinyl-2-dimethylaminomethylbenzeneboronic acid and boronic moieties described in U.S. Pat. Nos. 6,927,246 and 6,858,592 and incorporated herein by reference. Further examples of glucose binding moieties include those described in U.S. Pat. No. 6,916,660, which is also incorporated by the reference.

Aryl boronic acid compounds can also be reacted to form boronate esters with GAGs having free alcohol or diol groups. Reactions for forming boronate ester bonds are well known in the art and include refluxing the boronic acid and diol in an appropriate solvent (e.g., alcohol, toluene, methylene chloride, tetrahydrofuran or dimethyl sulfoxide). Alternatively, an aryl boronic acid can be added to a polymer.

### Grafting

The methods of generating a biomimetic proteoglycan discussed elsewhere herein is applicable to general grafting methodologies. Grafting copolymers contain side-chain branches emanating from different points along the polymer backbone. Variations in the nature of the main chain and side chains, in the length and polydispersity of the backbone and branches as well as in graft density determine the properties of the resulting graft copolymer. These variables also relate to the synthetic complexity of preparing these copolymers.

Graft copolymers can generally be prepared by the “onto”, “through” and “from” grafting processes. In the “grafting onto” process, end-functionalized polymer chains are attached to the main chain of another polymer by coupling reactions with functional groups along its backbone. “Grafting onto” is interchangeable with “grafting to”.

The “grafting through” process is based on the synthesis of a well-defined macromonomer, followed by its copolymerization with a low molecular weight comonomer. Control over length and polydispersity can be achieved for both backbone and side chains using this methodology. The approach is characteristic of a multistep synthesis and the grafting density is associated with the reactivity ratios of the macromonomers.

The “grafting from” process is based on the synthesis of a macroinitiator containing suitable initiating groups along the backbone. The high initiator efficiency, the ability to manipulate initiator distribution along the main chain and the side chain length control afforded by living polymerization techniques makes the “grafting from” process an attractive option in the synthesis of well defined graft copolymers. The multiple advantages of the living radical polymerization (LRP) is related to its ability to control molecular weight and polydispersity as well as water tolerance.

In one embodiment, the biomimetic proteoglycan can be fabricated via the “grafting to” method wherein a GAG chain is grafted to a functional polymer. The functional polymer can be, but is not limited to, any polymer with diol or primary amine reactive groups such as boronic acids epoxides, aldehyhdes and carboxylic acids. An example of a possible “grafted to” polymer is poly(acrylic acid) which is a carboxylic acid linear polymer chain which is subsequently activated with EDC/NHS and then reacted with CS via it's terminal primary amine creating a bottle brush structure.

In another embodiment, the biomimetic proteoglycan can be fabricated via the “grafting through” method wherein a GAG chain is modified with a polymerizable end group which is subsequently homo- or co-polymerized to form a bottle brush polymer. An example of a possible “grafted through” polymer occurs wherein 2-Vinyloxirane is attached to GAG chain via an interaction of the terminal primary amine in the GAG chain with the epoxide of 2-Vinyloxirane creating a vinyl-GAG. The vinyl-GAG is subsequently polymerized via free radical polymerization. Similarly another example is the attachment of poly(4-vinylbenzylboronic acid) to a GAG via an interaction of the terminal diol in the GAG with the boronic acid in poly(4-vinylbenzylboronic acid) forming a boronic ester. The vinylized-GAG is then subsequently polymerized via free radical polymerization to form a bottle brush polymer.

In some instances, “grafting-through” can be used for purposes of a step-growth polymerization. For example, grafting through via chain growth polymerization can be achieved using a free-radical strategy. Alternatively, grafting through via step-growth polymerization can be achieved using a di-epoxide strategy.

In another embodiment, the biomimetic proteoglycan can be fabricated via the “grafting from” method wherein a disaccharide unit of a GAG chain (e.g., GlcUA and GalNAc) is attached to a polymeric backbone via but not limited to aldehyde or amine interactions. Subsequent disaccharide or saccharide units are then grown from the polymeric backbone using enzymes of GAG synthesis such as but not limited to GlcA I transferase, GlaNAc transferase, chondroitin synthase, chondroitin 6-0 sulfotransferase and chondroitin 4-O-sulfotransferase.

In another embodiment, the biomimetic proteoglycan fabricated via any of the grafting methods disclosed elsewhere herein is end-functionalized with but not limited to a hyaluronan binding region or collagen binding region. Polymerizations that can be utilized to incorporate a functional group on the terminal end of the biomimetic proteoglycan bottle brush include but are not limited to radical polymerization, cationic polymerization, living anionic polymerization, atom transfer radical polymerization, and ring opening metathesis polymerization.

In one embodiment, the biomimetic proteoglycan is resistant to enzymatic digestion, so that the composition can be maintained over a period of time without breakdown. This provides the advantage that different components of the biomimetic proteoglycan can be repeatedly added onto an existing structure. Therefore, a large macromolcular sized biomimetic proteoglycan can be maintained in tissue over time.

In another embodiment, the biomimetic proteoglycan comprises a GAG chain that is modified. For example, the GAG chain can be modified to incorporate other functional elements such as tags for visualization or peptides for cellular recognition.

### Biomimetic Aggrecan

Aggrecan, which is one of the most widely studied proteoglycans, is abundant in cartilage; it represents up to 10% of the dry weight of cartilage. Many individual monomers of aggrecan bind to hyaluronic acid to form an aggregate, it is the monomer which is termed aggrecan. These aggregates are comprised of up to 100 monomers attached to a single chain of hyaluronic acid (HA).

An aggrecan monomer is believed to have a protein backbone of about 210-250 kDa to which is attached both chondroitin sulfate and keratan sulfate chains. The chains are attached to the central portion of the core protein, chondroitin sulfate chains (100-150 per monomer), being located in the C-terminal 90%, while the keratan sulfate (30-60 per monomer) is preferentially located towards the N-terminus.

Individual aggrecan monomers, up to about 100, interact with hyaluronic acid to form an aggregate of very high molecular weight. This interaction involves a globular domain at the N-terminus, termed G1 or the hyaluronic acid binding region (HABR). The interaction is stabilized by a short protein called link protein which interacts with both the HA and G1. This concentration of aggregated aggrecan is greatly diminished after about age 20.

The role of aggrecan in part relates to a physical element of the disc, as it brings about an osmotic swelling and electrostatic repulsion and maintains the high levels of hydration in the extracellular matrix. In this way, aggrecan plays a crucial role in the normal function of intervertebral discs. The presence on aggrecan of a very large numbers of chondroitin sulfate chains generates an osmotic swelling pressure. A preferred material of the invention is aggrecan or a material that mimics aggrecan. As used herein, “aggrecan” also refers to a biomimetic aggrecan composition. The present invention relates to the development of a biomimetic replacement for a ubiquitous biomacromolecule (e.g., aggrecan) for use as a minimally invasive early interventional technique for the treatment and prevention of back pain originating from intervertebral disc degeneration (IVD).

The disclosure presented herein demonstrates that restoration of healthy glycosaminoglycan levels in the nucleus pulposus of the intervertebral disc drastically changes the stress profile of the nucleus pulposus. The restoration of normal stress distributions in the IVD helps to prevent the propagation of remodeling and the degenerative cascade. A strategy for the replacement of GAG is the minimally-invasive introduction of biomimetic aggrecan analogues. These analogues are designed to mimic the organization of chondroitin sulfate in native aggrecan molecules. For example, the ability to attach chondroitin sulfate to various monomers or polymers via a primary amine interaction that is likely only available in the terminal region of the chondroitin sulfate molecule. This allows for the controlled organization of chondroitin sulfate onto various polymeric backbones that may be tuned to match the properties desired for mechanical restoration of the degenerated IVD.

The invention provides a biomimetic aggrecan useful for treating back pain. The invention provides a medical augmentation device wherein biomimetic aggrecan is administered to the site of injury or an adjacent site. The biomimetic aggrecan is based on the 3D brush-like structure of aggrecan (the primary proteoglycan of the nucleus of the intervertebral disc). Aggrecan has two main mechanical functions in the disc: 1) it allows water uptake by the nucleus due to sulfated groups in the chondroitin and keratan sulfate rich regions which, in part, provide intradiscal pressure and 2) it provides electrostatic repulsion due to the 3D macromolecular structure, which contributes to intradiscal pressure and disc height. However, the invention should not be limited to biomimetic aggrecan. Rather, the invention encompasses any biomimetic proteoglycan to treat a disease, disorder, or condition associated with a defective of dysfunctional proteoglycan.

As a non limiting example, the biomimetic aggrecan is generated by attaching chondroitin sulfate to a polymer. For example, the biomimetic aggrecan can be formed by the attachment of a terminal diol in chondroitin sulfate to a boronic acid polymer. Utilizing the high affinity complexation of boronic acids with compounds containing diols (such as saccharides), a novel polymer system has been developed to generate biomimetic aggrecan. For example, a free radical polymerization technique which comprises using a boronic acid functionalized polymer core to attach chondroitin sulfate to form brush “bristles” to mimic the bristles of the aggrecan molecule. The applied engineering of the polymer structure using a biomimetic philosophy enables the development of an effective early stage treatment to the spine.

In another embodiment, the biomimetic aggrecan of the invention can be generated by attaching at least chondroitin sulfate through a terminal primary amine handle to a diverse array of polymer backbones. This technique is based on attaching chondroitin sulfate to various monomers or polymers via a primary amine interaction that is likely only available in the terminal region of the chondroitin sulfate molecule. This allows for the controlled organization of chondroitin sulfate onto various polymeric backbones that may be tuned to match the properties desired for mechanical restoration of the degenerated IVD.

In one embodiment, the biomimetic aggrecan can be fabricated via a grafting method wherein chondroitin sulfate or other similar GAG chain is grafted to a functional polymer. The functional polymer can be, but is not limited to, any polymer with diol or primary amine reactive groups such as boronic acids epoxides, aldehydes and carboxylic acids. An example of a possible “grafted to” polymer is poly(acrylic acid) which is a carboxylic acid linear polymer chain which is subsequently activated with EDC/NHS and then is reacted with chondroitin sulfate via it's terminal primary amine creating a bottle brush structure.

In another embodiment, the biomimetic aggrecan is fabricated via the “grafting through” method wherein the chondroitin sulfate or other similar GAG chain is modified with a polymerizable end group which is subsequently homo- or co-polymerized to form a bottle brush polymer. An example of a possible “grafted through” polymer occurs wherein 2-Vinyloxirane is attached to chondroitin sulfate via an interaction of the terminal primary amine in chondroitin sulfate with the epoxide of 2-Vinyloxirane creating a vinyl chondroitin sulfate. The vinyl chondroitin sulfate is subsequently polymerized via free radical polymerization. Similarly another example is the attachment of poly(4-vinylbenzylboronic acid) to chondroitin sulfate via an interaction of the terminal diol in chondroitin sulfate with the boronic acid in poly(4-vinylbenzylboronic acid) forming a boronic ester. The vinylized chondroitin sulfate is then subsequently polymerized via free radical polymerization to form a bottle brush polymer.

In another embodiment, the biomimetic aggrecan is fabricated via the grafting from method wherein a disaccharide unit of chondroitin sulfate (GlcUA and GalNAc) or other GAG is attached to a polymeric backbone via but not limited to aldehyde or amine interactions. Subsequent disaccharide or saccharide units are then grown from the polymeric backbone using enzymes of GAG synthesis such as but not limited to GlcA I transferase, GlaNAc transferase, chondroitin synthase, chondroitin 6-O sulfotransferase and chondroitin 4-O-sulfotransferase.

In another embodiment, the biomimetic aggrecan fabricated via any of the grafting methods is end-functionalized with but not limited to a hyaluronan binding region or collagen binding region. Polymerizations that can be utilized to incorporate a functional group on the terminal end of the biomimetic aggrecan bottle brush include but are not limited to radical polymerization, cationic polymerization, living anionic polymerization, atom transfer radical polymerization, and ring opening metathesis polymerization.

In one embodiment, the biomimetic aggrecan is resistant to enzymatic digestion, so that the composition can be generated over a period of time without breakdown. This provides the advantage that different components of the biomimetic aggrecan can be repeatedly added onto an existing structure. Therefore, a large macromolecular sized biomimetic aggrecan can be generated over time.

In some instances, the size of the biomimetic aggrecan can be controlled so that a desired size is generated. In certain instances, this has the advantage that certain sizes are large enough to be unable to migrate out of the nucleus pulposus and/or disc. Chondroitin sulfate, keratan sulfate and other GAGs can migrate thereby limiting their use as compared with the biomimetic aggrecan of the invention.

In one embodiment, biomimetic aggrecan is arranged in the bottle-brush structure such that the electrostatically charged bristle molecules are in close proximity to one another. The close proximity of the charged bristles will provide electrostatic repulsions and steric hindrances that will assist the biomimetic aggrecan in resisting force. This will allow for two mechanisms of tissue restoration, an increased osmotic potential as well as mechanical function. In some instances, if the GAG chains are arranged in close proximity on the biomimetic aggrecan, the GAG chains can produce electrostatic repulsions which can contribute to the mechanical resistance of the biomimetic aggrecan.

In some instances, the electrostatic repulsions between closely packed GAG chains generate a mechanical resistance to force, thereby restoring mechanical function to the disc. Thus, the biomimetic aggrecan can be generated to exhibit both a desirable mechanical property as well as a desirable osmotic pressure when place into the disc of a mammal in need thereof.

In addition to the ability to generate desired sizes of biomimetic aggrecan, it is also possible according to the present invention to generate biomimetic aggrecan that is variably susceptible to enzymatic digestion. In some instance, it is desired that the biomimetic aggrecan is susceptible to enzymatic digestion. In other instances, it is desired that the biomimetic is resistant to enzymatic digestion.

In some instances, the present invention includes administering a material into the nucleus pulposus of a degenerated disc for the purpose of increasing the osmotic potential of the disc can restore disc height and function. It is believed that the osmotic pressure of the material added increases the overall osmotic potential of the nucleus material. Preferably, the osmotic pressure of the material is low enough that the resultant increase in pressure does not in itself cause pain. It is desirable to increase the osmotic pressure of the disc. Any increase in osmotic pressure that can restore disc height and function is encompassed in the invention

Whether the aggrecan is natural or a biomimetic aggrecan, the material of the invention can also be any combination of components making up aggrecan. For example, any combination of proteoglycan, HA, chondroitin sulfate, keratan sulfate, and the like can be administered into the nucleus pulposus. In some instances, the aggrecan administered into the disc can assemble on HA and form an aggrecan aggregate.

It will be understood from the present invention that other glycosaminoglycans and polysaccharides can be used for forming a biomimetic aggrecan. For example, suitable glycosaminoglycans, include HA, chondroitin, chondroitin sulfate, dermatan sulfate, heparan sulfate, keratan sulfate and heparin. In addition, any polymer that resembles a glycosaminoglycan can be used to generate the biomimetic aggrecan of the invention. Based on the disclosure presented herein, a skilled artisan would understand that any hydrophilic polymer can be used.

The aggrecan material and/or components thereof of the invention can be prepared using any method disclosed herein. For example, the materials can be isolated from a healthy donor. Preferably, the supply of aggrecan and/or components thereof can be derived from a mammal, preferably a human. The aggrecan and/or components thereof can be autologous, allogenic, or xenogenic with respect to the recipient. Alternatively, the materials can be produced by a cell. In another aspect, the materials can be produced synthetically.

In addition to aggrecan, the invention is applicable to produce any biomimetic proteoglycan. As a non-limiting example, versican is a large proteoglycan of about 265 KDa with 12-15 chondroitin sulfate chains attached. This protein is a major component of the dermal layer of skin, and interacts with hyaluronan in the extracellular matrix through N-terminal contacts. Versican also interacts with numerous other signaling molecules through C-terminal contacts. The central domain of versican contains the glycosaminoglycan attachment points, but differential splicing in various tissues leads to a variety of glycosaninoglycan attachments and sulfation patterns, further yielding an assortment of glycosaminoglycan chain interactions with other molecules. In addition, since versican is known to interact with hyaluronan, increased versican production may increase hyaluronan production.

In addition to versican, dermis contains several small leucine-rich proteoglycans (SLRPs) such as decorin, biglycan and lumican. SLRPs plays an important role in the regulation of cell activity and in the organization and functional properties of skin connective tissue. A modification of their repartition might be involved in the alterations which occur in skin aging. It was shown that lumican expression decreased during aging whereas decorin expression tended to increase, resulting in a strong alteration of the decorin to lumican ratio. Alterations of SLRPs expression could be implicated in the functional impairment which affect aged skin (Vuillermoz, et al., Mol Cell Biochem 277(1-2): 63-72, 2005).

Lumican has a 38 KDa protein core that contains two keratan sulfate GAG attachment sites, and has been shown to affect the integrity of the extracellular matrix and skin structure. For instance, knockout mice that could not express lumican displayed abnormal collagen assembly and brittle skin, suggesting lumican plays a large role in ECM maintenance and in skin health (Wegrowski et al., Mol Cell Biochem 205(1-2): 125-31, 2000; Vuillermoz, et al., Mol Cell Biochem 277(1-2): 63-72, 2005). Periodontal health is also affected by lumican removal due to its interactions with collagen (Matheson, et al., J Periodontal Res 40(4): 312-24, 2005). In addition, Roughley et al., (1996 Biochem J. 318:779) indicated a role for lumican and other SLRPs in protecting collagen from degradation by collagenases, further suggesting a role for lumican in ECM maintenance and prevention of ECM degradation (Geng, et al., Matrix Biol., 25(8):484-91 2006). Further, Vuillermoz et. al. showed that lumican expression decreased in skin fibroblasts with increased age, suggesting a possible role of lumican in age-related damage to skin. In addition, several studies have suggested that lumican plays a role in corneal health, as decreased or knocked-out lumican expression resulted in poor corneal formation (Chakravarti, Glycoconj J 19(4-5): 287-93, 2002), further supporting a role in collagen fibril formation, but, also, purified lumican has been shown to promote corneal epithelial wound healing (Yeh, et al. Opthalmol Vis Sci 46(2): 479-86, 2005). Therefore, it is likely that delivery of biomimetic lumican to skin would facilitate collagen fibril formation and increase the water content due to the charge and hydrophilicity of the glycosaminoglycan chains, thereby increasing skin health and appearance. Other known proteoglycans include syndecans 1-4, glypicans 1-5, betaglycan, NG2/CSPG4, CD44/epican, fibromodulin, PRELP, keratocan, osteoadherin/osteomodulin, epiphycan, osteoglycin/mimecan, neurocan/CSPG3, brevican, bamacan, agrin, and serglycin.

### Treatment of the Spine

The compositions of the invention are useful for treatment of the spine, in particular, for functional restoration of the disc in the spine.

The intervertebral disc comprises three major components: 1) the nucleus pulposus, 2) the annulus fibrosus, and 3) a pair of cartilaginous endplates. The present invention may be practiced upon any of these sites, alone or in any combination.

The nucleus pulposus typically contains more than 80 volume percent (vol %) water (depending on age and condition). The protein content of the nucleus pulposus typically comprises approximately 50 weight percent (wt %) proteoglycans, 20 wt % collagen (mainly Type II collagen), and other small proteins such as fibronectin, thromospondin, and elastin. The water and proteoglycan content of the nucleus pulposus generally decreases with age and onset of pathological changes. Hence, they are expected to be present in lower amounts in the intervertebral discs in patients that are candidates for the method of this invention.

The annulus fibrosis is generally slightly less hydrated than the nucleus pulposus and its protein content comprises about 15 wt % proteoglycan and 70 wt % collagen (mainly Type I collagen). The annulus fibrosis may also lose water with age and disease, but generally experiences more structural changes, such as tearing and formation of thick bundles, than biochemical changes.

The cartilaginous endplate is a thin layer of hyaline cartilage similar to articular cartilage and dry weight is composed of mainly Type II collagen.

In a healthy intervertebral disc, cells within the nucleus pulposus produce an extracellular matrix (ECM) containing a high percentage of proteoglycans. These proteoglycans contain sulfated functional groups that retain water, thereby providing the nucleus pulposus with its cushioning qualities.

Degeneration of an intervertebral disc occurs through damage to the nucleus pulposus tissue of the disc, which can be caused by aging, repetitive loading, or a significant overload. The severity of clinically observable disc degeneration varies widely from bulging, herniated and ruptured discs to advanced spondylosis leading to spinal stenosis, spondylolithesis and scoliosis. Patients suffering from a degenerated disc may experience a number of symptoms, including pain of the lower back, buttocks and legs, and sciatica.

The compositions and methods of the present invention can be used to treat individuals suffering from degenerated intervertebral disc conditions. The present invention is directed to compositions and methods for the repair of degenerated or damaged intervertebral discs through restoration of osmotic potential in the intervertebral disc. By administering a composition comprising aggrecan and/or components thereof into the intervertebral space of a degenerated disc, the damaged tissue can effectively be repaired.

The present invention provides less invasive procedures than those of the prior art for treatment of intervertebral disc disorders. In addition, the compositions and methods of the present invention can prompt biological repair of normal tissue in the disc, which results in better long term results than those obtained with synthetic prostheses. Administration of a material of the present invention into the degenerated disc can restore normal disc height and function. For example, the material of this invention can assist in the restoration of the load-bearing and viscoelastic properties of the defective intervertebral disc. The present invention can be used in conjunction with any known or heretofore unknown method of treating a disc disease or condition in a mammal, preferably a human. For example, the biomimetic aggrecan can be added to an adjuvant for fusion or be used in total disc arthroplasty (TDA) in adjacent discs. In addition, the biomimetic aggrecan can be used in adjacent discs after vertebroplasty due to compression fracture. In addition, the biomimetic aggrecan can be used for reconstruction in spondilolishesis or scholiosis.

The present invention includes administering aggrecan and/or any form thereof to a degenerative disc to restore at least the physical element of the disc. Other proteins that are useful for the invention include, but are not limited to hyaluronan, chondroitin sulfate, keratan sulfate, albumin, elastin, fibrin, fibronectin, and casein.

Preferably, the nucleus pulposus portion of the intervertebral disc is selected as the target site for the administration of aggrecan and/or components thereof. Treating the nucleus pulposus with agrrecan and/or components thereof can stiffen the nucleus pulposus (thereby reducing undesired mobility).

In some embodiments, both the nucleus pulposus and the annulus fibrosis may be, treated with the same administration of aggrecan and/or components thereof. In other embodiments, only the annulus fibrosis is treated.

In another embodiment of this invention, a non-enzymatic polysaccharide oxidizing agent is injected in combination with aggrecan and/or components thereof into the nucleus pulposus of a pathological intervertebral disc. Because the dry weight component of the nucleus pulposus is rich in proteoglycans, there are numerous sites that can be oxidized to form functional aldehydes. Subsequently, the aldehydes can react with amino acid regions of both native and non-native collagens and proteoglycans to form a network of molecules.

In another aspect, the aggrecan is attached to a polymer backbone such as polyethylene glycol or polyvinyl alcohol or other HA analog. The backbone is used to implant aggrecan into the intervertebral disc. The backbone is also useful for providing structure to prevent aggrecan and/or components thereof from migrating out of the intervertebral disc (e.g., the nucleus space).

To facilitate administration, the aggrecan can be delivered in a carrier. The carrier can be water or another liquid in which aggrecan is soluable. Likewise a liquid can be one in which the aggrecan does not dissolve. One such a liquid is a biocompatible oil. The concentration of aggrecan in the carrier can be such that that the volume of material administered, carrier and aggrecan, either swells or contracts in vivo. The idea is to administer a specific amount of aggrecan sufficient to restore function of the disc. Preferably, the material swells in vivo. This means that the aggrecan concentration must be below its capacity to adsorb and hold water in the nucleus environment. It is believed that such a concentration would not be flowable. In such case the non-solvent carrier can be used. The non-solvent carrier can migrate out of the disc space and allow the aggrecan to swell. If the required concentration was not flowable, a fraction of the desired concentration can be used and administered into the disc of successive days or weeks to build the desired concentration in the disc space, for example, one third concentration would require three administrations. The method may include a single administration or a series of administrations in order obtain desired disc restoration.

Without wishing to be bound by any particular theory, it is believed that the aggrecan solution should contain a clinically relevant amount of aggrecan. As used herein, “aggrecan” also refers to a biomimetic aggrecan composition. Clinically relevant can be determined by measuring the concentration of aggrecan in health disc and measuring in degenerated disc. The difference would theoretically be the amount needed. This concentration should be available in a volume that could be administered in a disc. The aggrecan can be administered with no disc preparation or some material can be removed to make appropriate room for the aggrecan. Aggrecan can also be packaged as a dry substance that can be reconsitituted prior to use.

Another method of accomplishing the same goal of restoring the load carrying capability of the disc includes administering proteoglycan, HA, chondroitin and keratan sulfate and allowing the components to self aggregate to form aggrecan in the disc space in vivo. These components can also be modified with proteins to facilitate their self agglomeration. The components can be xenograft, allograft or synthetic or could be analogs thereof.

### Treatment of Other Soft Tissue Defects and Disorders

The compositions disclosed herein may be used to treat any number of soft tissue disorders and defects in a manner similar to that described for the treatment of the spine. For example, functional restoration of cartilage and/or the meniscus in the knee may be accomplished by administering the compositions of the invention to the knee. Similarly, soft tissue disorders and defects in other body tissues, including, but not limited to skin, heart valve, articular cartilage, fatty tissue, craniofacial, ocular, tendon, ligament, fascia, fibrous tissue, synovial membrane, muscle, nerves, and blood vessel. Disorders or defects in any one of these sites may be treated by administering the compositions of the invention to the respective site. Thus, the invention should be construed to include treatment of soft tissue defects and disorders to effect functional restoration of the same. The precise methods to be used will be readily apparent to the skilled artisan with experience in the soft tissue in question.

### Cellular Compositions

The invention also includes the use of viable cells in combination with the biomimetic proteoglycan. Examples of such cells include harvested cells selected from the group consisting of healthy nucleus pulposus or annulus fibrosus cells, precursors of nucleus pulposus or annulus fibrosus cells, or cells capable of differentiating into nucleus pulposus or annulus fibrosus cells. In some instances, the biomimetic proteoglycan can be used as a cell matrix for supporting both in vivo as well as in vitro cell culture.

Also included in the invention is a hybrid material in which cells are combined with the biomimetic proteoglycan of the invention. Intervertebral disc cells may be isolated from tissue extracted from any accessible intervertebral disc of the spine. For example, tissue may be extracted from the nucleus pulposus of lumbar discs, sacral discs or cervical discs. Preferably, intervertebral disc cells are primarily nucleus pulposus cells. In some embodiments, it is preferred that disc cells are at least 50% nucleus pulposus cells while 90% nucleus pulposus cells is still more preferred. Cells may be obtained from the patient being treated, or alternatively cells may be extracted from donor tissue.

The following methods can be used, in some embodiments of the invention, to isolate and culture disc cells including but not limited to precursor and/or nucleus pulposus cells. Nucleus pulposus and/or annulus fibrosus tissue is removed from intervertebral discs using methods known to those skilled in the art. The tissues are treated with collagenase at about 37° C. at a concentration of about 0.1 unit/ml to about 10 unit/ml, and more preferably at about 1 unit/ml, for about 15 minutes to about 2 hours. Following collagenase treatment, the cells are swollen and easily ruptured, and are gently pipetted up and down to break up the aggregates. The cell suspensions are centrifuged at about 2500 rpm for about 5 min. The supernatant is discarded and the cell pellet is suspended in complete Dulbecco's Eagle's Medium supplemented with about 1% to about 70% fetal calf serum, and more preferably about 10% fetal calf serum, about 0.1 mM to about 20 mM, and more preferably about 2 mM, glutamine and penicillin/streptomycin/fungicide. The cells are treated with hylauronidase (about 0.1 unit/ml to about 10 unit/ml, and more preferably about 1 unit/ml) to facilitate cell attachment and are washed with complete medium, that is, medium containing 10% serum, to remove the hylauronidase.

In some embodiments of the invention, nucleus pulposus and/or precursor cells are selected after hyaluronidase treatment, thereby separating them from non-nucleus pulposus and/or non-precursor cells, using methods familiar to the skilled artisan, such as, for example, FACS. In some embodiments of the invention, non-nucleus pulposus or non-precursor cells are removed after hylauronidase treatment using methods familiar to the skilled artisan, such as, for example, elutration, which involves differential centrifugation based upon the buoyant density of the cells, or centrifugation over a Percoll gradient.

In another embodiment of the invention, the precursor and/or nucleus pulposus cells are isolated by gently teasing out fragments of nucleus pulposus tissue from intervertebral discs. The tissue is placed in culture vessels with tissue culture medium and cells are allowed to grow out from the nucleus pulposus tissue. In about 7 to 14 days, the cells are released from the tissue culture plastic and collected by centrifugation. In some embodiments of the invention, nucleus pulposus and/or precursor cells are selected after collection by centrifugation.

In the event that intervertebral disc cells are not available, the invention includes the use of any cell that is capable of differentiating into a disc cell. Other cells that are useful include cells that are capable of producing aggrecan and/or components thereof. For example, stem cells can be used to generate the desired material. Stem cells include, but are not limited to embryonic stem cells and adult stem cells derived or obtained from any source, preferably a human source.

In another aspect of the invention, the desired cells may be allogeneic with respect to the recipient. The allogeneic cells are isolated from a donor that is a different individual of the same species as the recipient. Following isolation, the cells are cultured using standard culturing methods to produce an allogeneic product. The invention also encompasses cells that are xenogeneic with respect to the recipient.

Any appropriate medium capable of supporting cell culture may be used to culture the cells of the invention. Media formulations that support the growth of cells include, but are not limited to, Minimum Essential Medium Eagle, ADC-1, LPM (bovine serum albumin-free), F10 (HAM), F12 (HAM), DCCM1, DCCM2, RPMI 1640, BGJ Medium (with and without Fitton-Jackson Modification), Basal Medium Eagle (BME—with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E—with Earle's salt base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA with non-essential amino acids), and the like.

Additional non-limiting examples of media useful in the methods of the invention can contain fetal serum of bovine or other species at a concentration at least 1% to about 30%, preferably at least about 5% to 15%, most preferably about 10%. Embryonic extract can be present at a concentration of about 1% to 30%, preferably at least about 5% to 15%, most preferably about 10%.

In some embodiments of the invention, the medium is supplemented with fibronectin at about 0.0001 to about 1 mg/ml, including any and all whole or partial increments therebetween. In some embodiments of the invention, the medium is supplemented with TGF-β at about 10 picograms/ml to about 10,000 picograms/ml, including any and all whole or partial increments therebetween, and more preferably at about 100 picograms/ml to about 1000 picograms/ml, including any and all whole or partial increments therebetween; with PDGF at about 1.0 ng/ml to about 10,000 ng/ml, including any and all whole or partial increments therebetween, and more preferably at about 10 ng/ml to about 1000 ng/ml, including any and all whole or partial increments therebetween; with EGF at about 0.5 ng/ml to about 150 ng/ml, including any and all whole or partial increments therebetween, and more preferably at about 1.0 ng/ml to about 10 ng/ml, including any and all whole or partial increments therebetween; with FGF at about 0.5 ng/ml to about 150 ng/ml, including any and all whole or partial increments therebetween, and more preferably at about 1.0 ng/ml to about 10 ng/ml, including any and all whole or partial increments therebetween; with IL-1 at about 0.5 ng/ml to about 150 ng/ml, including any and all whole or partial increments therebetween, and more preferably at about 1.0 ng/ml to about 10 ng/ml, including any and all whole or partial increments therebetween; and with IL-6 at about 0.5 ng/ml to about 150 ng/ml, including any and all whole or partial increments therebetween, and more preferably at about 1.0 ng/ml to about 10 ng/ml, including any and all whole or partial increments therebetween. The medium is replenished every 2-4 days.

Following isolation, the cells of the invention are incubated in the desired cell medium in a culture apparatus for a period of time or until the cells reach confluency before passing the cells to another culture apparatus. The culturing apparatus can be any culture apparatus commonly used in culturing cells in vitro. Preferably, the level of confluence of the cells is greater than 70% before transferring the cells to another culture apparatus. More preferably, the level of confluence is greater than 90%. A period of time can be any time suitable for the culture of cells in vitro. Cell medium may be replaced during the culture of the cells at any time. Preferably, the cell medium is replaced every 2 to 4 days. Cells are then harvested from the culture apparatus whereupon the cells can be used immediately or cryopreserved and stored for use at a later time. Cells may be harvested using trypsinization, EDTA treatment, or any other procedure used to harvest cells from a culture apparatus.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to the seeding density, substrate, medium, and time between passaging.

### Genetic Modification

The cells of the present invention can also be used to express a foreign protein or molecule for a therapeutic purpose or to generate aggrecan and/or components thereof. Thus, the invention encompasses expression vectors and methods for the introduction of exogenous DNA into the cells with concomitant expression of the exogenous DNA in the cells. Methods for introducing and expressing DNA in a cell are well known to the skilled artisan and include those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

The term “genetic modification” as used herein refers to the stable or transient alteration of the genotype of a cell by intentional introduction of exogenous DNA. The DNA may be synthetic, or naturally derived, and may contain genes, portions of genes, or other useful DNA sequences. The term “genetic modification” as used herein is not meant to include naturally occurring alterations such as that which occurs through natural viral activity, natural genetic recombination, or the like.

Exogenous DNA may be introduced to a cell using viral vectors (retrovirus, modified herpes viral, herpes-viral, adenovirus, adeno-associated virus, lentiviral, and the like) or by direct DNA transfection (lipofection, calcium phosphate transfection, DEAE-dextran, electroporation, and the like).

One purpose of genetic modification of the cell is for the production aggrecan and/or components thereof. However, the cells can also be genetically modified for the purpose of producing of a biological agent. Examples of biological agents include, but are not limited to, chemotactic agents; therapeutic agents (e.g., antibiotics, steroidal and non-steroidal analgesics and anti-inflammatories (including certain amino acids such as glycine), anti-rejection agents such as immunosuppressants and anti-cancer drugs); various proteins (e.g., short term peptides, bone morphogenic proteins, collagen, hyaluronic acid, glycoproteins, and lipoprotein); cell attachment mediators; biologically active ligands; integrin binding sequence; ligands; various growth and/or differentiation agents and fragments thereof (e.g., epidermal growth factor (EGF), hepatocyte growth factor (HGF), vascular endothelial growth factors (VEGF), fibroblast growth factors (e.g., bFGF), platelet derived growth factors (PDGF), insulin derived growth factor (e.g., IGF-1, IGF-II) and transforming growth factors (e.g., TGFβ I-III), parathyroid hormone, parathyroid hormone related peptide, bone morphogenic proteins (e.g., BMP-2, BMP-4; BMP-6; BMP-7; BMP-12; BMP-13; BMP-14), sonic hedgehog, growth differentiation factors (e.g., GDF5, GDF6, GDF8), recombinant human growth factors (e.g., MP52, and MP-52 variant rhGDF-5), cartilage-derived morphogenic proteins (CDMP-1; CDMP-2, CDMP-3)); small molecules that affect the upregulation of specific growth factors; tenascin-C; hyaluronic acid; chondroitin sulfate; fibronectin; decorin; thromboelastin; thrombin-derived peptides; heparin-binding domains; heparin; heparan sulfate.

A preferred bioactive agent is a substance that is useful for the treatment of a given bone disorder. For example, it may be desired to genetically modify cells so that they secrete a certain growth factor product associated with bone formation.

The cells of the present invention can be genetically modified by introducing exogenous genetic material into the cells to produce a molecule such as a trophic factor, a growth factor, a cytokine, and the like. In addition, the cell can provide an additional therapeutic effect to the mammal when transplanted into a mammal in need thereof. For example, the genetically modified cell maybe modified to secrete a molecule that is beneficial to neighboring cells in the mammal and ultimately cause a beneficial effect in the mammal.

As used herein, the term “growth factor product” refers to a protein, peptide, mitogen, or other molecule having a growth, proliferative, differentiative, or trophic effect on a cell. Specific growth factors useful in the treatment of bone disorders include, but are not limited to, FGF, TGF-β, insulin-like growth factor, and bone morphogenetic protein.

According to some aspects of the invention, cells obtained from the mammal to be treated or from another donor mammal, may be genetically altered to replace a defective gene and/or to introduce a gene whose expression has therapeutic effect in the mammal being treated.

In all cases in which a gene construct is transfected into a cell, the heterologous gene is operably linked to regulatory sequences required to achieve expression of the gene in the cell. Such regulatory sequences typically include a promoter and a polyadenylation signal.

The gene construct is preferably provided as an expression vector that includes the coding sequence for a heterologous protein operably linked to essential regulatory sequences such that when the vector is transfected into the cell, the coding sequence will be expressed by the cell. The coding sequence is operably linked to the regulatory elements necessary for expression of that sequence in the cells. The nucleotide sequence that encodes the protein may be cDNA, genomic DNA, synthesized DNA or a hybrid thereof, or an RNA molecule such as mRNA.

The gene construct includes the nucleotide sequence encoding the beneficial protein operably linked to the regulatory elements and may remain present in the cell as a functioning cytoplasmic molecule, a functioning episomal molecule or it may integrate into the cell's chromosomal DNA. Exogenous genetic material may be introduced into cells where it remains as separate genetic material in the form of a plasmid. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be introduced into the cell.

The regulatory elements for gene expression include: a promoter, an initiation codon, a stop codon, and a polyadenylation signal. It is preferred that these elements be operable in the cells of the present invention. Moreover, it is preferred that these elements be operably linked to the nucleotide sequence that encodes the protein such that the nucleotide sequence can be expressed in the cells and thus the protein can be produced. Initiation codons and stop codons are generally considered to be part of a nucleotide sequence that encodes the protein. However, it is preferred that these elements are functional in the cells. Similarly, promoters and polyadenylation signals used must be functional within the cells of the present invention. Examples of promoters useful to practice the present invention include but are not limited to promoters that are active in many cells such as the cytomegalovirus promoter, SV40 promoters and retroviral promoters. Other examples of promoters useful to practice the present invention include but are not limited to tissue-specific promoters, i.e. promoters that function in some tissues but not in others; also, promoters of genes normally expressed in the cells with or without specific or general enhancer sequences. In some embodiments, promoters are used which constitutively express genes in the cells with or without enhancer sequences. Enhancer sequences are provided in such embodiments when appropriate or desirable.

The cells of the present invention can be transfected using well known techniques readily available to those having ordinary skill in the art. Exogenous genes may be introduced into the cells using standard methods where the cell expresses the protein encoded by the gene. In some embodiments, cells are transfected by calcium phosphate precipitation transfection, DEAE dextran transfection, electroporation, microinjection, liposome-mediated transfer, chemical-mediated transfer, ligand mediated transfer or recombinant viral vector transfer.

In some embodiments, recombinant adenovirus vectors are used to introduce DNA with desired sequences into the cell. In some embodiments, recombinant retrovirus vectors are used to introduce DNA with desired sequences into the cells. In other embodiments, standard CaPO₄, DEAE dextran or lipid carrier mediated transfection techniques are employed to incorporate desired DNA into dividing cells. In some embodiments, DNA is introduced directly into cells by microinjection. Similarly, well-known electroporation or particle bombardment techniques can be used to introduce foreign DNA into the cells. A second gene is usually co-transfected or linked to the therapeutic gene. The second gene is frequently a selectable antibiotic-resistance gene. Standard antibiotic resistance selection techniques can be used to identify and select transfected cells. Transfected cells are selected by growing the cells in an antibiotic that will kill cells that do not take up the selectable gene. In most cases where the two genes co-transfected and unlinked, the cells that survive the antibiotic treatment contain and express both genes.

It should be understood that the methods described herein may be carried out in a number of ways and with various modifications and permutations thereof that are well known in the art. It should also be appreciated that any theories set forth as to modes of action or interactions between cell types should not be construed as limiting this invention in any manner, but are presented such that the methods of the invention can be more fully understood.

### Administration

The compositions of the present invention may be administered to a soft tissue site in a mammal, for the functional restoration thereof, using a variety of methods and in a variety of formulations known in the art. The mammal is preferably a human.

In some instances, it is preferable that the composition of the invention does not appreciably degrade following administration. In other instances, it is preferred that the composition of the invention degrades either rapidly, or slowly, in the tissue. Thus, when administered in the body, a biomimetic proteoglycan, such as aggrecan, may be permanent, may be degraded enzymatically, or may be degraded in the presence of a solvent, such as, for example, water.

The compositions of the present invention can take the form of immediate release (injection) formulations, or delayed release formulations, i.e., using microspheres, nanospheres or other matrices such as hydrogels for controlled release. When administered to a disc, and recognizing that the methods and formulations disclosed herein are equally applicable to other tissues, it is envisioned that any suitable annular closure technique may be used before or after insertion of aggrecan (and/or components) thereof into the disc tissue. The annular closure technique can be applied before or after administration. Examples of suitable closure techniques may include the use of the following alone or in combination, sutures (resorbable or non-resorbable strips/cords/draw strings/wires/cords), adhesives (fibrin, cyanoacrylates, polyanhydrides, glutaraldehydes, PRP, etc.), in-situ fabricated plugs (single sheet wound or two piece snapped together), pre-fabricated plugs (like a tire plug), expandable plugs (stent like), for example.

Delivery of the desired material into the nucleus pulposus or annulus fibrosus of the disc may be by delivery through the ruptured area of the annulus, by delivery a separate passageway way through or into the annulus, or by delivery through a plug or other closure device used to repair the ruptured annulus. Delivery of the material can also be accomplished by direct administration into the nucleus pulposus.

In accordance with the present invention there is provided a method for restoring a damaged or degenerated intervertebral disc comprising administering an administerable formulation comprising aggrecan (and/or components thereof). The administerable formulation can either be viscous or form a solid or gel in situ.

In another embodiment of the present invention, the administerable formulation is an aqueous solution. In a preferred embodiment, the administerable formulation comprises an aqueous solution containing a biopolymer such as a cellulosic, a polypeptidic or a polysaccharide or a mixture thereof. One preferred biopolymer is chitosan, a natural partially N-deacetylated poly(N-acetyl-D-glucosamine) derived from marine chitin. Other preferred biopolymers include collagen (of various types and origins). Other biopolymers of interest include methyl cellulose, hydroxyethyl cellulose, hydroxypropyl methyl cellulose, and the like.

In the preferred embodiments of this invention, the administerable formulation preferably comprises an aqueous solution containing a water-soluble dibasic phosphate salt. The administerable formulation may contain a mixture of different water-soluble dibasic phosphate salts. The preferred dibasic phosphate salts comprise dibasic sodium and magnesium monophosphate salts as well as monophosphate salt of a poly or sugar. This does not exclude the use of water-soluble dibasic salts other then phosphate, such as carboxylate, sulfate, sulfonate, and the like. Other preferred formulations of the method may contain hyaluronic acid or chondroitin sulfate or synthetic polymers such poly(ethylene glycol) or poly(propylene glycol), and the like.

In other embodiments of this invention, the administerable in situ setting formulation is nonaqueous (does not contain water) and solid or gel forming (turns into a solid or gel in situ).

In another embodiment of this invention, the administerable formulation is nonaqueous and comprises an organic solvent or a mixture of organic solvents. Metabolically absorbable solvents are preferably selected (triacetin, ethyl acetate, ethyl laurate, etc).

In another embodiment of this invention, the administerable formulation is nonaqueous and contains at least one fatty acid or a mixture of fatty acids. The administerable formulation comprises saturated or unsaturated fatty acid selected from the group consisting of palmitate, stearate, myristate, palmitoleate, oleate, vaccenate and linoleate. It may be a mixture of several of these fatty acids. The fatty acid may be mixed with a metabolically absorbable solvent or liquid vehicle to reduce viscosity and allow administerability.

In yet another embodiment, the administerable formulation is a dry powder, which when introduced into the soft tissue, e.g., the disc, is hydrated within the tissue to result in the desired restoration thereof.

In the method of the present invention, a low viscosity formulation is administered into degenerated disc. It is mixable with the nucleus chemical and biological materials, and preferably forms a gel or solid in situ. The formulation is administered easily, with a minimal pressure, through the fine tube of a needle or catheter. Typical tube gauge ranges are from 13 to 27. Administrations are performed by instruments or devices that provide an appropriate positive pressure, e.g. hand-pressure, mechanical pressure, injection gun, etc. One representative technique is to use a hypodermic syringe.

In another embodiment, the formulation is administered by injection through the wall of intact annulus fibrosus into the nucleus pulposus.

The invention also includes a method of administering aggrecan and/or components thereof by way of simple injection through a needle preferably 18 gauge or smaller or a small cannula, preferably 2 mm or less in diameter The preferred administration site is at the posterior, lateral or posterio-lateral region of the disc and is accomplished through. It is envisioned that the aggrecan and/or components thereof can be pre-packaged sterilely in syringes for easy and safe use.

An advantage of the present invention is that the entire intervertebral disc is not removed in order to effect treatment of the degenerated disc. However, it is recognized that in some instances, the materials of the present invention can be administered into the degenerated disc without removing native material from the degenerated disc prior to administration of the materials. The purpose of removing native material from the degenerated disc is to make room for the materials to be administered.

When cells are used to treat a degenerated disc, the cells may be administered to a mammal following a period of in vitro culturing. The cell may be cultured in a manner that induces the cell to differentiate in vitro. However, the cells can be administered into the recipient in an undifferentiated state where the administered cells differentiate to express at least one characteristic of a disc cell in vivo in the mammal.

The cells of this invention can be transplanted into a mammal using techniques known in the art such as i.e., those described in U.S. Pat. No. 5,618,531, which is incorporated herein by reference, or into any other suitable site in the body. Transplantation of the cells of the present invention can be accomplished using techniques well known in the art as well as those described herein, or using techniques developed in the future. The present invention comprises a method for transplanting, grafting, infusing, or otherwise introducing the cells into a mammal, preferably, a human.

The cells can be suspended in an appropriate diluents. Suitable excipients for administration solutions are those that are biologically and physiologically compatible with the cells and with the recipient, such as buffered saline solution or other suitable excipients. The composition for administration can be formulated, produced and stored according to standard methods complying with proper sterility and stability.

The cells may also be encapsulated and used to deliver biologically active molecules, according to known encapsulation technologies, including microencapsulation (see, e.g., U.S. Pat. Nos. 4,352,883; 4,353,888; and 5,084,350, herein incorporated by reference), or macroencapsulation (see, e.g., U.S. Pat. Nos. 5,284,761; 5,158,881; 4,976,859; and 4,968,733; and International Publication Nos. WO 92/19195; WO 95/05452, all of which are incorporated herein by reference). For macroencapsulation, the number of cells used in the devices can be varied. Several macroencapsulation devices may be administered in the mammal. Methods for macroencapsulation and administration of cells are well known in the art and are described in, for example, U.S. Pat. No. 6,498,018.

The mode of administration of the cells of the invention to the mammal may vary depending on several factors including the type of disease being treated, the age of the mammal, whether the cells are differentiated or not, whether the cells have exogenous DNA introduced therein, and the like. The cells may be introduced to the desired site by direct administration, or by any other means used in the art for the introduction of compounds administered to a mammal suffering from a particular disease or disorder of the disc.

The invention further provides, in some aspects, methods of treating a degenerative disc by administering a composition comprising a cell, a matrix, a cell lysate, a cell-product of the invention (i.e. molecules secreted by the cell), or any combination thereof in a mammal in need thereof. As such, the invention encompasses a pharmaceutical composition, wherein the composition may be used in the treatment of a bone condition such as a degenerated disc.

In a non-limiting embodiment, a formulation comprising a cell, a matrix, a cell lysate, a cell-product of the invention (i.e. molecules secreted by the cell), or any combination thereof is prepared for administration directly to the degenerated disc. For example, the cells of the invention may be suspended in a hydrogel solution for administration. Alternatively, the hydrogel solution containing the cells may be allowed to harden, for instance in a mold, to form a matrix having cells dispersed therein prior to administration, or once the matrix has hardened, the cell formations may be cultured so that the cells are mitotically expanded prior to administration. The hydrogel is an organic polymer (natural or synthetic) which is cross-linked via covalent, ionic, or hydrogen bonds to create a three-dimensional open-lattice structure which entraps water molecules to form a gel. Examples of materials which can be used to form a hydrogel include polysaccharides such as alginate and salts thereof, peptides, polyphosphazines, and polyacrylates, which are crosslinked ionically, or block polymers such as polyethylene oxide-polypropylene glycol block copolymers which are crosslinked by temperature or pH, respectively.

In some embodiments, the polymers are at least partially soluble in aqueous solutions, such as water, buffered salt solutions, or aqueous alcohol solutions, that have charged side groups, or a monovalent ionic salt thereof. Examples of polymers with acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

Examples of polymers with basic side groups that can be reacted with anions are poly(vinyl amines), poly(vinyl pyridine), poly(vinyl imidazole), and some imino substituted polyphosphazenes. The ammonium or quaternary salt of the polymers can also be formed from the backbone nitrogens or pendant imino groups. Examples of basic side groups are amino and imino groups.

Other examples of polymers include, but are not limited to poly-alpha-hydroxy esters, polydioxanone, propylene fumarate, poly-ethylene glycol, poly-erthoesters, polyanhydrides and polyurethanes, poly-L-lactic acid, poly-glycolic acid, and poly-lactic-co-glycolic acid.

Transplantation of Cells Using Scaffolds

The present invention includes using the biomimetic proteoglycan as a component of a scaffold to deliver cells to the desired tissue. The cells can be seeded onto or into a three-dimensional scaffold and administered in vivo in a mammal, where the seeded cells proliferate on the framework and form a replacement tissue in vivo in cooperation with the cells of the mammal.

In some aspects of the invention, the scaffold comprises extracellular matrix, cell lysate (e.g., soluble cell fractions), or combinations thereof, of the desired cells. In some embodiments, the scaffold comprises an extracellular matrix protein secreted by the cells of the invention. Alternatively, the extracellular matrix is an exogenous material selected from the group consisting of calcium alginate, agarose, fibrin, collagen, laminin, fibronectin, glycosaminoglycan, hyaluronic acid, heparin sulfate, chondroitin sulfate A, dermatan sulfate, and bone matrix gelatin. In some aspects, the matrix comprises natural or synthetic polymers.

The invention includes biocompatible scaffolds, lattices, self-assembling structures and the like, whether biodegradable or not, liquid or solid. Such scaffolds are known in the art of cell-based therapy, surgical repair, tissue engineering, and wound healing. Preferably the scaffolds are pretreated (e.g., seeded, inoculated, contacted with) with the cells, extracellular matrix, conditioned medium, cell lysate, or combination thereof. In some aspects of the invention, the cells adhere to the scaffold. The seeded scaffold can be introduced into the mammal in any way known in the art, including but not limited to implantation, injection, surgical attachment, transplantation with other tissue, injection, and the like. The scaffold of the invention may be configured to the shape and/or size of a tissue or organ in vivo. For example, but not by way of limitation, the scaffold may be designed such that the scaffold structure supports the seeded cells without subsequent degradation; supports the cells from the time of seeding until the tissue transplant is remodeled by the host tissue; and allows the seeded cells to attach, proliferate, and develop into a tissue structure having sufficient mechanical integrity to support itself.

Scaffolds of the invention can be administered in combination with any one or more growth factors, cells, drugs or other and/or components described elsewhere herein that stimulate tissue formation or otherwise enhance or improve the practice of the invention. The cells to be seeded onto the scaffolds may be genetically engineered to express growth factors or drugs.

In another preferred embodiment, the cells of the invention are seeded onto a scaffold where the material exhibits specified physical properties of porosity and biomechanical strength to mimic the features of natural bone, thereby promoting stability of the final structure and access and egress of metabolites and cellular nutrients. That is, the material should provide structural support and can form a scaffolding into which host vascularization and cell migration can occur. In this embodiment, the desired cells are first mixed with a carrier material before application to a scaffold. Suitable carriers include, but are not limited to, calcium alginate, agarose, types I, II, IV or other collagen isoform, fibrin, poly-lactic/poly-glycolic acid, hyaluronate derivatives, gelatin, laminin, fibronectin, starch, polysaccharides, saccharides, proteoglycans, synthetic polymers, calcium phosphate, and ceramics (i.e., hydroxyapatite, tricalcium phosphate).

The external surfaces of the three-dimensional framework may be modified to improve the attachment or growth of cells and differentiation of tissue, such as by plasma coating the framework or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratan sulfate), a cellular matrix, and/or other materials such as, but not limited to, gelatin, alginates, agar, and agarose.

In some embodiments, it is important to re-create in culture the cellular microenvironment found in vivo. In addition, growth factors, osteogenic inducing agents, and angiogenic factors may be added to the culture medium prior to, during, or subsequent to inoculation of the cells to trigger differentiation and tissue formation by the cells following administration into the mammal.

### Therapeutic Applications

The present invention encompasses methods for administering a composition comprising a biomimetic aggrecan, a cell, a matrix, a cell lysate, a cell-product of the invention (i.e. molecules secreted by the cell), or any combination thereof to a degenerative disc. Preferably, the composition contains at least biomimetic aggrecan and/or components thereof. Biomimetic aggrecan and/or components thereof may be administered alone or as admixtures with other cells and/or a bioactive factor as discussed elsewhere herein.

When the composition comprises a cell, the skilled artisan will readily understand that the cells can be transplanted into a mammal whereby upon receiving signals and cues from the surrounding milieu, the cells differentiate into mature cells in vivo dictated by the neighboring cellular milieu. Preferably, the cells differentiate into a cell that exhibits at least one characteristic of a disc cell. Alternatively, the desired cells can be differentiated in vitro into a desired cell type and the differentiated cell can be administered to a mammal in need thereof.

The compositions of the invention may be surgically implanted, injected, delivered (e.g., by way of a catheter or syringe), or otherwise administered directly or indirectly to the site in need of repair, restoration, or augmentation. The compositions may be administered by way of a matrix (e.g., a three-dimensional scaffold). The compositions may be administered with conventional pharmaceutically acceptable carriers.

To enhance the differentiation, survival or activity of administered cells, additional bioactive factors as discussed elsewhere herein may be added. For example, a bioactive factor can include, but is not limited to bone morphogenetic protein, vascular endothelial growth factor, fibroblast growth factors, and other cytokines that have either osteoconductive and/or osteoinductive capacity. To enhance vascularization and survival of transplanted bone tissue, angiogenic factors such as VEGF, PDGF or bFGF can be added either alone or in combination with endothelial cells or their precursors.

Alternatively, the cells to be transplanted may be genetically engineered to express such growth factors, antioxidants, antiapoptotic agents, anti-inflammatory agents, or angiogenic factors.

The result of administering the materials of the present invention to a degenerated disc is to increase the osmotic potential in the degenerated disc. By administering a composition comprising biomimetic aggrecan and/or components thereof into the intervertebral space of a degenerated disc, the damaged tissue can effectively be repaired. The methods of the present invention can be used in conjunction with any annulus repair technology.

Soft Tissue Restoration and/or Augmentation

The present invention also provides methods for soft tissue restoration and/or augmentation in a subject comprising, administering a composition of the present invention to a mammal in need thereof. The method of the invention is designed to improve conditions including, but not limited to, lines, folds, wrinkles, minor facial depressions, cleft lips, correction of minor deformities due to aging or disease, deformities of the vocal cords or glottis, deformities of the lip, crow's feet and the orbital groove around the eye, breast deformities, chin deformities, cheek and/or nose deformities, acne, surgical scars, scars due to radiation damage or trauma scars, and rhytids. The soft tissue can also be located in the pelvic floor, in the periurethral area, near the neck of the urinary bladder, or at the junction of the urinary bladder and the ureter. The method of soft tissue augmentation may increase tissue volume. The compositions may be administered into the skin or may be administered underneath the skin. The compositions include insoluble elastin derived from human vascular tissue that does not induce inflammatory or immune response and does not induce calcification.

Restoration, repair and/or augmentation of soft tissue, such as skin, can be an important factor in recovering from injury or for cosmetic purposes. For example, with normal aging, skin may become loose or creases can form, such as nasal-labial folds. In the face, creases or lines may adversely affect a person's self esteem or even a career. Thus, there has been a need for compositions and methods that can diminish the appearance of creases or lines.

Further, there are situations in which loss of tissue can leave an indentation in the skin. For example surgical removal of a dermal cyst, lipoatrophy or solid tumor can result in loss of tissue volume. In other cases, injuries, such as gunshot wounds, knife wounds, or other excavating injures may leave an indentation in the skin. Regardless of the cause, it can be desirable to provide adermal filler that can increase the volume of tissue to provide a smoother or more even appearance.

One example for needed support is dermal restoration, repair and/or augmentation in the face where dermal and subdermal volume is lost due to aging.

The term “soft tissue augmentation” includes, but is not limited to, the following: dermal tissue augmentation; filling of lines, folds, wrinkles, minor facial depressions, cleft lips and the like, especially in the face and neck; correction of minor deformities due to aging or disease, including in the hands and feet, fingers and toes; augmentation of the vocal cords or glottis to rehabilitate speech; hemostatic agent, dermal filling of sleep lines and expression lines; replacement of dermal and subcutaneous tissue lost due to aging; lip augmentation; filling of crow's feet and the orbital groove around the eye; breast augmentation; chin augmentation; augmentation of the cheek and/or nose; bulking agent for periurethral support, filling of indentations in the soft tissue, dermal or subcutaneous, due to, e.g., overzealous liposuction or other trauma; filling of acne or traumatic scars and rhytids; filling of nasolabial lines, nasoglabellar lines and infraoral lines.

The term “augmentation” means the repair, decrease, reduction or alleviation of at least one symptom or defect attributed due to loss or absence of tissue, by providing, supplying, augmenting, or replacing such tissue with the composition of the present invention. The compositions of the present invention can also be used to prevent at least one symptom or defect in the tissue.

Dermal fillers are used to fill scars, depressions and wrinkles. Dermal filler substances have various responses in the dermis from phagocytosis to foreign body reactions depending on the material (Lemperle et al., Aesthetic Plast. Surg. 27(5):354-366; discussion 367 (2003)). One goal of dermal fillers is to temporarily augment the dermis to correct the surface contour of the skin without producing an unacceptable inflammatory reaction, hypersensitivity reaction or foreign body reaction that causes pain, redness or excessive scar formation for a period of time.

The ideal material for human skin augmentation would include one or more of the critical extracellular matrix elements that provide skin its mechanical properties. These elements include collagen, elastin and glycosaminoglycans. In addition, to obviate immune responses, these materials should optimally be of human origin. Human materials will also induce less inflammatory reaction than animal-derived materials, and hence will be likely to persist longer after administration into the recipient, thereby extending and improving the cosmetic effect of a formulation suitable for administration.

Many types of dermal filling procedures can benefit from the use of the compositions of the present invention. The uses of the present invention are designed (but not limited) to be used to provide increased volume of a tissue that, through disease, injury or congenital property, is less than desired. Compositions can be made to suit a particular purpose, and have desired retention times and physical and/or chemical properties.

Exemplary uses of compositions of this invention can be particularly desirable to fill facial tissue (e.g., nasolabial folds), to increase the volume of the dermis in the lips, nose, around the eyes, the ears and other readily visible tissue. Additionally, the compositions can be desirably used to provide bulk to increase the volume of skin secondary to excavating injuries or surgeries. For example, the site around a dermal cyst can be filled to decrease the appearance of a dimple at the site of surgery.

As such, the present invention provides methods of skin augmentation by administering the compositions of the invention to a subject in need thereof. Preferably, the methods improve skin wrinkles and/or increase skin volume. The subject or patient treated by the methods of the invention is a mammal, more preferably a human. The following properties or applications of these methods will essentially be described for humans although they may also be applied to non-human mammals, e.g., apes, monkeys, dogs, mice, etc. The invention therefore can also be used in a veterinarian context.

### Combination Therapy

The biomimetic proteoglycan can be administered to a mammal in need therefore alone or in combination with additional components including but not limited to hyaluronic acid, a hyaluronic acid analog or collagen.

In one embodiment, the biomimetic proteoglycan can be combined with a biomolecule (such as a nucleic acid, amino acid, sugar or lipid). Such a biomolecule can be covalently attached or non-covalently associated with the biomimetic proteoglycan described herein. In an exemplary embodiment, the biomolecule is a member selected from a receptor molecule, extracellular matrix component or a biochemical factor. In another exemplary embodiment, the biochemical factor is a member selected from a growth factor and a differentiation factor.

In another exemplary embodiment, the biomimetic proteoglycan of the invention can be combined with a first molecule (which may or may not be a biomolecule). Such a first molecule can be covalently attached to the biomimetic proteoglycan of the invention. This first molecule can be used to also interact with a biomolecule discussed above. In an exemplary embodiment, the first molecule is a linker, and the second biomolecule is a member selected from a receptor molecule, biochemical factor, growth factor and a differentiation factor. In an exemplary embodiment, the first molecule is a member selected from heparin, heparan sulfate, heparan sulfate proteoglycan, and combinations thereof. In an exemplary embodiment, the second biomolecule is a member selected from a receptor molecule, biochemical factor, growth factor and a differentiation factor. In another exemplary embodiment, the first molecule is covalently attached through a linker, and said linker is a member selected from di-amino poly(ethylene glycol), poly(ethylene glycol) and combinations thereof. For biomolecules that do not bind to heparin, direct conjugation to the polymer scaffold or through a linker (such as PEG, amino-PEG and di-amino-PEG) is also feasible. In another exemplary embodiment, the biomolecule is an extracellular matrix component which is a member selected from laminin, collagen, fibronectin, elastin, vitronectin, fibrinogen, polylysine, other cell adhesion promoting polypeptides and combinations thereof. In another exemplary embodiment, the biomolecule is a growth factor which is a member selected from acidic fibroblast growth factor, basic fibroblast growth factor, nerve growth factor, brain-derived neurotrophic factor, insulin-like growth factor, platelet derived growth factor, transforming growth factor beta, vascular endothelial growth factor, epidermal growth factor, keratanocyte growth factor and combinations thereof. In another exemplary embodiment, the biomolecule is a differentiation factor which is a member selected from stromal cell derived factor, sonic hedgehog, bone morphogenic proteins, notch ligands, Wnt and combinations thereof.

The first molecules which are covalently attached to the biomimetic proteoglycan of the invention can be used to interact with a biomolecule (for example, a growth factor and/or ECM component) in order to stimulate cell growth. In another exemplary embodiment, the biomimetic proteoglycan can be used for wound healing, and the biomolecule which is a member selected from an extracellular matrix component, growth factors and differentiation factors. Examples of potential factors for wound healing enhancement include epidermal growth factor (EGF), vascular endothelial growth factor (VEGF), basic fibroblast growth factor (bFGF) and platelet-derived growth factor (PDGF).

Biomolecules can be incorporated within the compositions of the invention during fabrication or post-fabrication. These biomolecules can be incorporated via covalent attachment directly or through various linkers or by adsorption.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations which are evident as a result of the teachings provided herein.

Intervertebral disc (IVD) degeneration occurs with aging, and may be a major cause of back pain. Alterations to the composition of the major biochemical constituents of the IVD have been shown to coincide with aging and disc degeneration and can subsequently alter the discs' ability to support load. The most significant biochemical change that takes place in disc degeneration is the loss of proteoglycans in the nucleus pulposus (NP). As the larger aggregating proteoglycans such as aggrecan are degraded into smaller fragments they are able to leach more readily from the NP than their larger constituents resulting in a loss of the charged glycosaminoglycans (GAGs) which are covalently attached to the aggrecan core protein.

The following experiments were designed to investigate the role of proteoglycans on intervertebral disc osmotic potential and function. Experiments were also designed to investigate whether administering an osmotic material into the nucleus of a degenerated disc is sufficient to restore disc function. It is believed that administering a material into the nucleus of a degenerated disc and increasing its osmotic potential, normal disc height and function may be restored.

Osmotic pressure is the pressure that must be applied to a solution to prevent the inward flow of fluid, and is very sensitive to GAG concentration. It depends mainly on the concentration of fixed charges on the PGs (i.e. fixed charge density), as it arises from the Donnan distribution of ions between PGs and the external fluid. Swelling pressure, the pressure at which there is no driving force for fluid flow, results from the osmotic pressure exerted by the PGs and the resulting tension in the collagen network of the IVD, which tends to restrain the swelling tendencies of the PGs. At equilibrium, the osmotic pressure of the PGs is balanced by the tensile response in the collagen network, opposing swelling (Urban et al., 1981, Connect Tissue Res 9(1):1-10). The osmotic pressure of proteoglycans at concentrations found in NP tissues (0.18-0.35 meq/gH₂O, fixed charge density) has been determined to lie in the range of approximately 0.03 to 0.3 MPa (0.15M NaCl, 37° C.) (Urban et al., 1979, Biorheology 16:447-64).

The experiments discussed elsewhere herein were performed to investigate the effects of proteoglycan restoration on the stress distribution in the NP and annulus fibrosus (AF, outer region) of the IVD using an axisymmetric finite element model. Experiments were designed to determine the role of aggrecan and/or components thereof on the osmotic potential in a degenerative disc.

The results presented herein demonstrate that aggrecan, including biomimetic aggrecan and/or components thereof can increase the osmotic potential and mechanical properties of a degenerative disc and are therefore able to restore normal disc height and function.

### Example 1: Effect of Aging and Degeneration on Fluid Exchange, Stress Concentrations and Osmotic Pressure of the Human Intervertebral Disc During the Diurnal Cycle

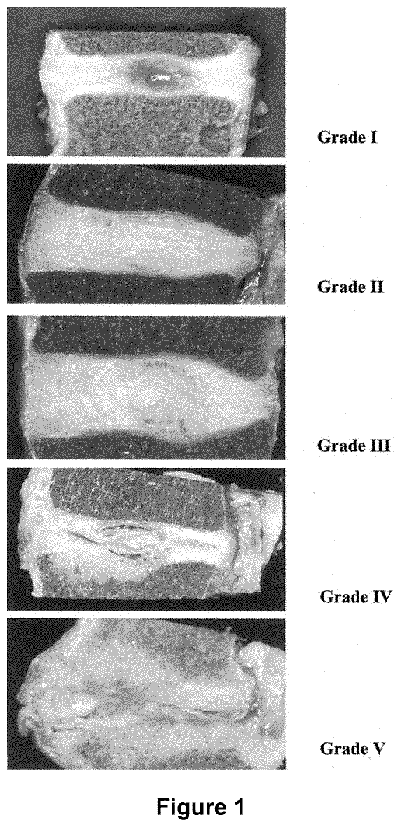

The human intervertebral disc is the primary compression-carrying component of the spine. Its roles are to transmit and distribute loads, and allow for the necessary flexibility of the spine. It is comprised of a central gel-like nucleus pulposus, an outer annulus fibrosus, and upper and lower endplates consisting of cartilaginous and bony portions. During a diurnal cycle, the intervertebral disc experiences approximately 16 hours of functional loading (standing, sitting, etc.), followed by 8 hours of recovery (lying prone). Therefore, the fluid lost during the loading period must be replenished in half the time. As the disc is compressed and fluid is exuded, the density of the fixed charges within the nucleus pulposus is increased, creating an osmotic gradient with the interstitial fluid surrounding the disc. This osmotic potential aids in drawing fluid back into the disc. The intervertebral disc has been shown to change with age and degeneration (Ayotte et al., 2000, Journal of Biomechanical Engineering 122(6):587-93; Buckwalter, 1995, Spine 20:1307-14; Friberg et al., 1949, Acta Orhtopaedica Scandinavica 19:222-42; Iatridis et al., 1997, Journal of Orthopaedic Research 15:318-22; Iatridis et al., 1998, Journal of Biomechanics 31(6):535-44; Johannessen et al., 2005, Spine 30(24):E724-E9; Miller et al.k, 1988, Spine 13(2):173; Roughley, 2004, Spine 29(23):2691-9; Urban et al., 1988, Spine (Phila Pa. 1976) 13(2):179-87). Alterations in the major biochemical constituents of the intervertebral disc have been shown to coincide with aging and disc degeneration, and can subsequently alter the discs' ability to support load. A significant biochemical change that takes place in disc degeneration is the loss of proteoglycans in the central region of the disc, the nucleus pulposus. Proteoglycans work to resist mechanical forces in the nucleus and, through hydration of the molecules, provide a hydrostatic pressure to the outer layers of the disc, the annulus fibrosus. In a dehydrated disc, the function of the nucleus, namely load transfer to the annulus through creation of an intradiscal pressure, is no longer occurring at a normal level. The mechanics of the degenerated disc are clearly altered compared to those of the intact disc. Degeneration is measured through the Thompson grading scale of the state of the tissue, as seen in FIG. 1 (Thompson et al., 1990, Spine 15(4):411-5).

Individual tissues can be tested to assess the change with degeneration, but experimental testing is limited in its ability to assess the complex ionic and mechanical stress distributions throughout the disc tissues. Experimental testing also does not show the reactions in the interior of the disc. Finite element analysis can be a useful tool in analyzing the internal mechanical effects of aging and degeneration of the intervertebral disc. The finite element modeling software ABAQUS contains an internal procedure for the poroelastic model, which has been shown to be equivalent to the biphasic model provided that the fluid phase is inviscid and can be used accordingly (Bowen, 1980, Int J Engng Sci 18(9):1; Mow et al., 1980, Journal of Biomechanical Engineering 102:73-84; Simon, 1992, Applied Mechanics Reviews 45:191; Wu et al., 1998, Journal of Biomechanics 31:165-9). Wilson et al. utilized and adjusted the poroelastic theory in ABAQUS via user-defined materials to incorporate the effects of osmotic swelling in articular cartilage (Wilson et al., 2005, Journal of Biomechanical Engineering 127(1):158-65; Wilson et al., 2005, Journal of Biomechanics 38(6):1195-204; Wilson et al., 2004, Journal of Biomechanics 37(3):357-66). Exploiting the advantages of ABAQUS makes the modeling of swelling behavior simpler and computationally less expensive, while producing basically the same results as the more complex mechano-electrochemical (quadriphasic) models (Wilson et al., 2005, Journal of Biomechanical Engineering 127(1):158-65). The following experiments were designed to use an osmo-poroelastic model to analyze the effects of intervertebral disc degeneration on the diurnal mechanical response of the disc. Understanding these effects may aid in providing a solution to disc degeneration and the corresponding lower back pain.

The materials and methods employed in the experiments disclosed herein are now described.

### Model Construction and Validation

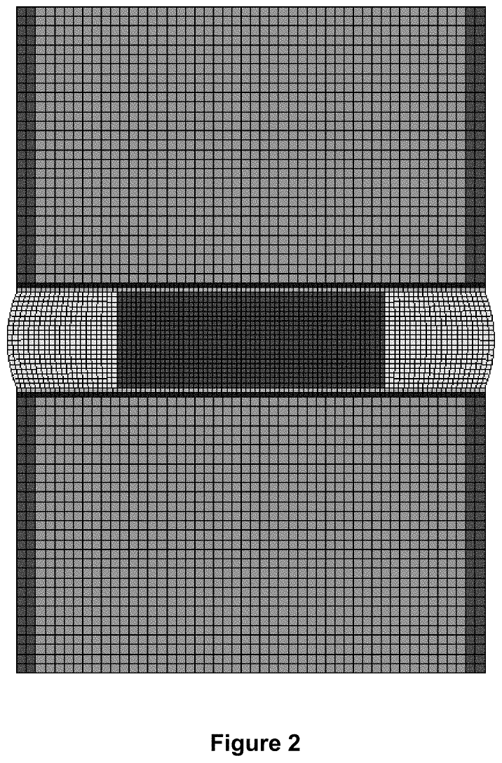

An axisymmetric, osmo-poroelastic model was created using ABAQUS v6.5 finite element software (SIMULIA, Providence, R.I.). The model consists of a nucleus pulposus, an annulus fibrosus, cartilaginous and bony portions of the adjacent endplates, and cancellous and cortical portions of the corresponding vertebrae FIG. 2. The standard poroelastic theory included in ABAQUS is utilized, but a user-defined material was incorporated to include the effects of osmotic swelling (Wilson et al., 2005, Journal of Biomechanical Engineering 127(1):158-65; Wilson et al., 2005, Journal of Biomechanics 38(6):1195-204). The model response was validated against experimental results such as axial displacement, radial displacement of the outer annulus fibrosus, and total fluid lost (Malko et al., 2002, Journal of Spinal Disorders & Techniques 15(2):157-63; Klein et al., 1983, Journal of Biomechanics 16(3):211-7; Heuer et al., 2008, Journal of Biomechanics 41(5):1086-94; Natarajan et al., 2003, Computers and Structures 81(8-11):835-42; Heuer et al., 2007, Clinical Biomechanics 23(3):260-9; Adams et al., 1996, Spine 21(4):434; Lu et al., 1996, Spine 21(19):2208; Malko et al., 1999, Spine 24(10):1015; Botsford et al., 1994, Spine 19(8):935; McMillan et al., 1996, British Medical Journal 55(12):880-7; Heuer et al., 2007, Clinical Biomechanics 22(7):737-44). The dimensions used in the model were approximations gathered from experimental results found in literature of typical lumbar discs—an initial disc height of 10 mm, an outer diameter of 24.5 mm, a nucleus diameter of 14 mm, total endplate height of 1 mm (0.5 mm for each of boney and cartilaginous portions), and vertebral body height of 29 mm. The outer 2 mm of the vertebrae is considered cortical bone, and the remainder is trabecular bone. The fibrous structure of the annulus fibrosus is simulated using tension-only structural rebar elements. An unloaded intervertebral disc bulges slightly; therefore a 1 mm bulge in the outer annulus at the axial midpoint was included. The model consists of 2626 4-node displacement and pore pressure (CAX4P) elements and 3091 nodes.

### Material Properties

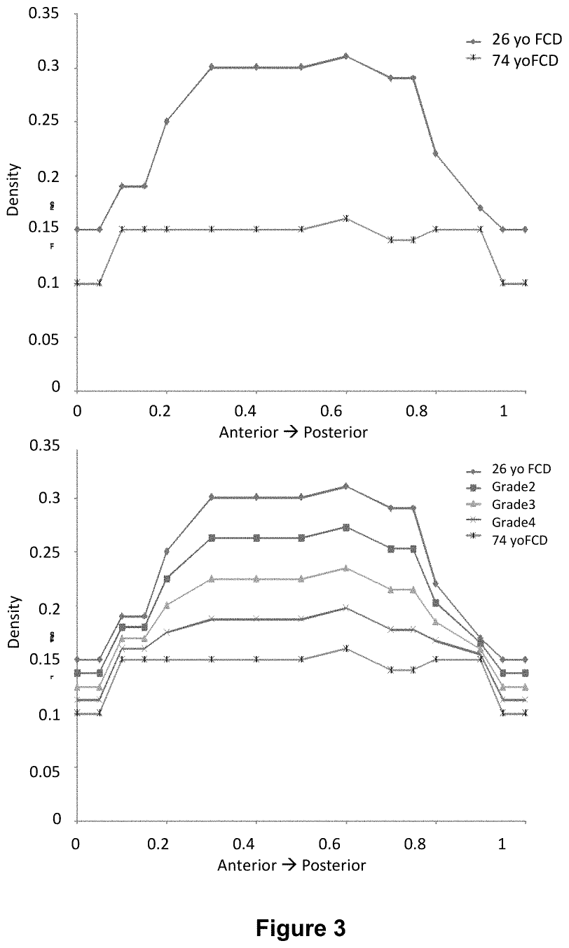

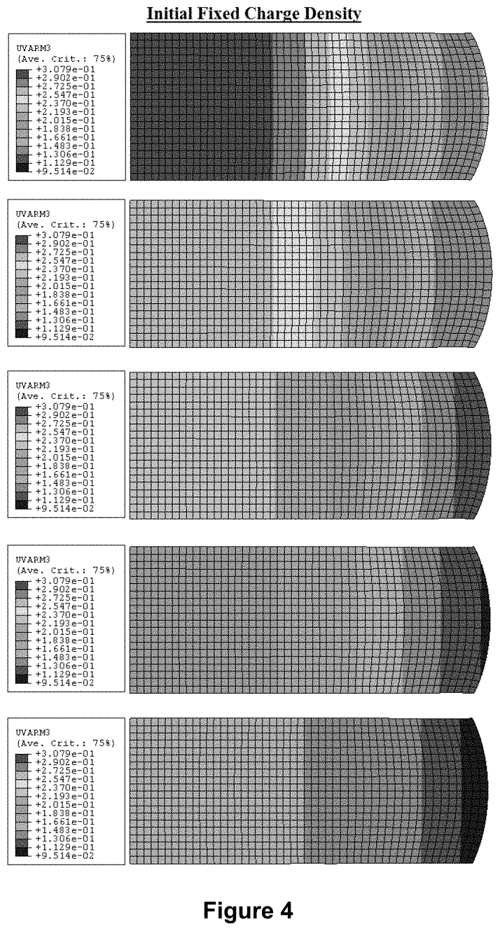

Grade 1 material properties-including those of the nucleus pulposus (Johannessen et al., 2005, Spine 30(24):E724-E9; Perie et al., 2005, Journal of Biomechanics 38(11):2164-71; Perie et al., 2006, Journal of Biomechanics 39(8):1392-400; Heneghan et al., 2008, Journal of Biomechanics 41(4):903-6), annulus fibrosus (Iatridis et al., 1998, Journal of Biomechanics 31(6):535-44; Perie et al., 2005, Journal of Biomechanics 38(11):2164-71; Drost et al., 1995, Journal of Biomechanical Engineering 117(4):390-6; Houben et al., 1997, Spine 22(1):7-16; Ebara et al., 1996, Spine 21(4):452-61; Fujita et al., 1997, Journal of Orthopaedic Research 15(6):814-9; Best et al., 1994, Spine 19(2):212-21; Acaroglu et al., 1995, Spine 20(24):2690-701; Smith et al., 2008, Annals of Biomedical Engineering 36(2):214-23; Elliott et al., 2001, Journal of Biomechanical Engineering 123(3):256-63; Elliott et al., 2000, Journal of Biomechanical Engineering 122(2):173-9; Gu et al., 1999, Spine 24(23):2449), cartilaginous endplate (Elliott et al., 2002, Journal of Biomechanical Engineering 124(2):223-8; Lai et al., 1981, Journal of Biomechanical Engineering 103:61-6; Setton, et al., 1993, Journal of Orthopaedic Research 11(2):228-39; Mansour et al., 1976, Journal of Bone and Joint Surgery 58-A(4):509-16; Mow et al., 1984, Journal of Biomechanics 17(5):377-294), bony endplate (Nauman et al., 1999, Annals of Biomedical Engineering 27(4):517-24), cortical bone (Nauman et al., 1999, Annals of Biomedical Engineering 27(4):517-24), and trabecular bone (Nauman et al., 1999, Annals of Biomedical Engineering 27(4):517-24)—were taken from literature Table 1. Degenerated material properties of the nucleus pulposus (Johannessen et al., 2005, Spine 30(24):E724-E9), annulus fibrosus (Iatridis et al., 1998, Journal of Biomechanics 31(6):535-44; Fujita et al., 1997, Journal of Orthopaedic Research 15(6):814-9; Acaroglu et al., 1995, Spine 20(24):2690-701; Smith et al., 2008, Annals of Biomedical Engineering 36(2):214-23), and boney endplates (Ayotte et al., 2000, Journal of Biomechanical Engineering 122(6):587-93) were also taken from literature. The remaining properties were interpolated from these values, as shown in Table 1. Fixed charge density profiles for healthy (grade 1) and degenerated (grade 5) are shown in FIG. 3A (Urban J P G, Holm S H. Intervertebral Disc Nutrition as Related to Spinal Movements and Fusion. In: AR H, editor. Tissue Nutrition and Viability. New York: Springer-Verlag; 1986. p. 101-19). Although the 26 year old disc may not be a grade 1, it is treated as such for the purpose of this study, as is the 74 year old as a grade 5. The profiles for grades 2-4 were linearly interpolated from these reported values, as seen in FIG. 3B. FIG. 4 shows the initial fixed charge density profiles as contour plots of the nucleus pulposus and annulus fibrosus for each degenerative grade.

TABLE 1
Finite element model material properties for Thompson degenerative grades 1-5

| | | Grade 1 | Grade 2 | Grade 3 | Grade 4 | Grade 5 |
|---|---|---|---|---|---|---|
| Nucleus | E [MPa] | 0.75 | 0.88 | 1.01 | 1.14 | 1.28 |
| Pulposus | □ | 0.17 | | | | |
| | e | 4.00 | | | | |
| | k [m\hat{ }4/(Ns)] | 1.00E−15 | 1.10E−15 | 1.2−15 | 1.30E−15 | 1.40E−15 |
| | FCD Profile | 1 | 2 | 3 | 4 | 5 |
| Annulus | E [MPa] | 1.50 | | 2.00 | 2.50 | 3.00 |
| Fibrosus | □ | 0.17 | | | | |
| | e | 2.33 | | | | |
| | k [m\hat{ }4/(Ns)] | 2.0E−16 | | | | |
| | FCD Profile | 1 | 2 | 3 | 4 | 5 |
| Annulus | E [MPa] | 100.00 | | | | |
| Fibers | □ | 0.10 | | | | |
| | Area [mm\hat{ }2] | 0.03 | | | | |
| Cartilaginous | E [MPa] | 5.00 | | | | |
| Endplate | □ | 0.17 | | | | |
| | e | 4.00 | | | | |
| | k [m\hat{ }4/(Ns)] | 1.43E−13 | | | | |
| | FCD [M] | 2.00E−01 | | | | |
| Bony | E [MPa] | 10000.00 | | | | |
| Endplate | □ | 0.30 | | | | |
| | e | 0.05 | | | | |
| | k [m\hat{ }4/(Ns)] | 1.00E−15 | 8.22E−16 | 6.43E−16 | 4.65E−16 | 2.86E−16 |
| | FCD [M] | 1.50E−01 | | | | |
| Cortical | E [MPa] | 10000.00 | | | | |
| Bone | □ | 0.30 | | | | |
| | e | 0.05 | | | | |
| | k [m\hat{ }4/(Ns)] | 7.00E−17 | | | | |
| | FCD [M] | 1.50E−01 | | | | |
| Trabecular | E [MPa] | 100.00 | | | | |
| Bone | □ | 0.20 | | | | |
| | e | 1.00 | | | | |
| | k [m\hat{ }4/(Ns)] | 2.00E−07 | | | | |
| | FCD [M] | 1.50E−01 | | | | |

### Loading and the Diurnal Cycle

The diurnal cycle is approximated as a 16 hour loading period, followed by an 8 hour recovery. The unit was loaded with a 0.5 MPa pressure on the upper vertebra to represent the functional loading experienced during daily activity, and a 0.1 MPa recovery load to simulate sleep conditions (Wilke et al., 1999, Spine 24(8):755-62). As is seen in experimental studies, a steady-state condition is found after several loading and recovery cycles due to the exchange of fluid (Johannessen et al., 2004, Annals of Biomedical Engineering 32(1):70-6). Therefore, each simulation consisted of four diurnal cycles, with the fourth cycle considered to be the steady-state cycle.

The results of the experiments are now described.

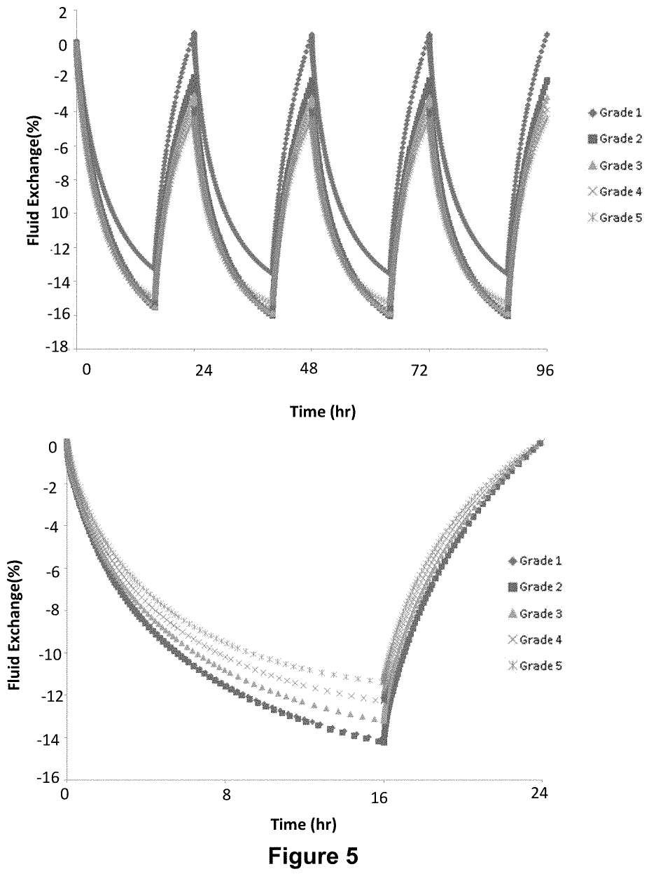

Total fluid lost during the first daily loading cycle is approximately 16% for each of the degenerated conditions, which is within the range found in literature of 10-20% (Malko et al., 2002, Journal of Spinal Disorders & Techniques 15(2):157-63; Malko et al., 1999, Spine 24(10):1015; Botsford et al., 1994, Spine 19(8):935). The steady-state fluid loss ranges from approximately 11% to approximately 14%, which is also within this range. Grade 1 actually absorbs more fluid during its initial recovery period than it lost during its initial loading period, leading to a positive fluid exchange at the end of the first cycle. Grades 2 through 5 lose approximately 2% to 4%, which remains unrecovered. The overall loss is approximately the same for Grades 2 through 5, with the highest fluid recovery value decreasing with degeneration. Note the grouping of the recovery curves (FIG. 5).

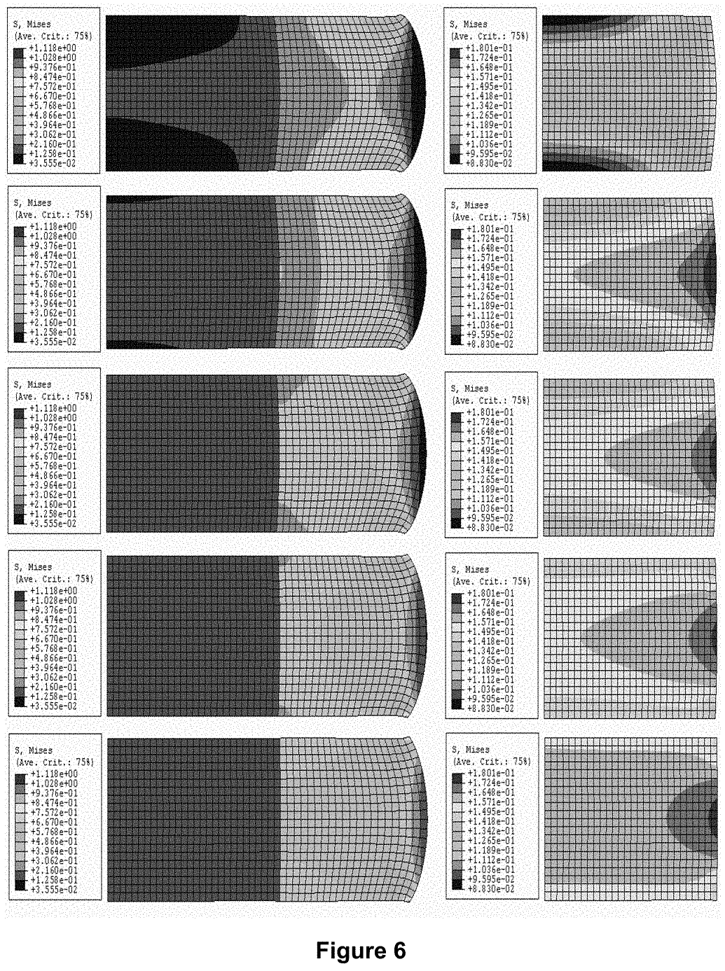

FIG. 6 shows the von Mises stress contour plots of the nucleus pulposus and annulus fibrosus combined, and the nucleus pulposus by itself. This stress value is the stress experienced by the tissue, which is found by taking the stress in the solid portion of the tissue less the osmotic pressure. Stress at the nucleus pulposus-annulus fibrosus interface increases with degeneration, as does the stress in the majority of the annulus fibrosus, from approximately 0.2 to approximately 0.4 MPa. There is an increase in the center of the nucleus pulposus from approximately 1.2 to approximately 1.6 MPa. Also, the nucleus pulposus side of the interface sees an increase from approximately 1.5 to approximately 1.8 MPa.

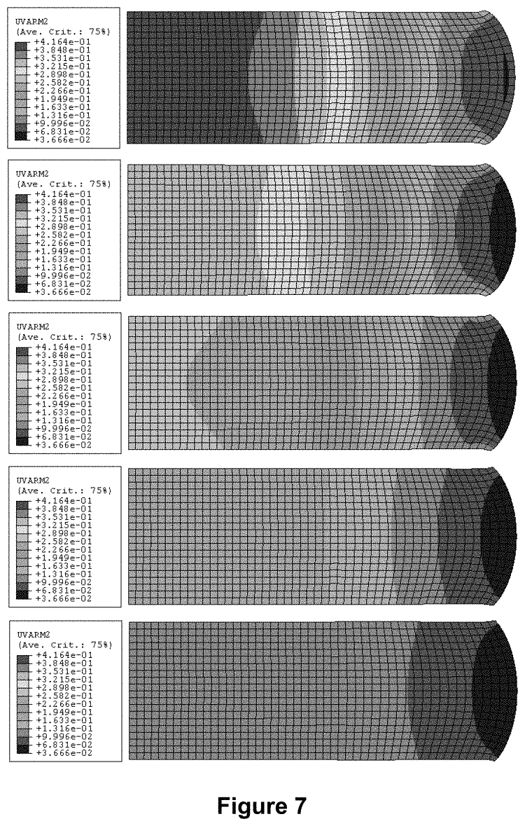

FIG. 7 shows the osmotic pressure of the disc in contour plot form. For each grade, the highest osmotic pressures are seen in the central nucleus pulposus, and decrease radially outwards towards the outer annulus fibrosus. The central nucleus pulposus decreases the most with degeneration, from approximately 0.42 MPa to approximately 0.1 MPa. These values are in the range of those seen in literature (Urban et al., 1980, Proceedings of the Institution of Mechanical Engineers. 2:63-9; Urban et al., 1985, Biorheology(Oxford). 22(2):145-57).

### Effect of Degeneration on Fluid Exchange

Fluid exchange is calculated using the voids ratio of the nucleus pulposus and annulus fibrosus. The voids ratio is defined as the ratio of the volume of voids to the volume of solid. After the initial elastic response, the volume of the solid maintains its value, while the volume of the voids decreases due to fluid being expelled from the tissue. When looking at all four cycles, the initial loading cycle for grade 1 loses almost 3% less fluid than grades 2 through 5, which are all nearly identical. This is due to the high osmotic pressure in grade 1, which in turn is due to the high initial fixed charge density in the nucleus pulposus. The differences in grades 2 through 5 are seen in the recovery period, where grade 2 recovers the most fluid while grade 5 recovers the least, almost a 3% difference. There is less fluid recovered at each grade for increasing levels of degeneration.

When looking at the steady-state cycle only, grade 1 and grade 2 are nearly identical, and the total fluid loss differences from grade 2 to grade 3, grade 3 to grade 4, and grade 4 to grade 5 are nearly linear at approximately 1% less fluid loss with each grade. The recoveries are nearly identical. Steady-state fluid loss decreases with degeneration because there is less fluid available as more is lost during the first three cycles.

### Effect of Degeneration on Stress

The annulus fibrosus sees the highest stress at its interface with the nucleus pulposus and at the outer corners, where the attachment to the cartilaginous endplates causes a high stress concentration. Both of these are artifacts of the model. The high concentration of stress at the nucleus pulposus-annulus fibrosus interface is likely an artifact of the abrupt change in material properties across the interface. In the actual tissue, there is a transition zone, which would prevent this by gradually changing properties. Also, in the actual tissue, the annulus fibrosus connects directly to the adjacent vertebrae, and the endplates are completely covered by the annulus fibrosus. There are also no sharp angles or edges in the actual tissue, which is a major cause of stress concentrations.

There is a gradual, steady increase in stress in the annulus with degeneration. When looking at the nucleus pulposus only, it is evident why the annulus fibrosus must remodel itself to account for the initial change in properties of the nucleus pulposus. The stress experienced by the nucleus pulposus increases greatly in grade 2 from grade 1, but then decreases in grade 3, and even grade 4 experiences lower stresses than in grade 2. By grade 5, however, the stresses in the nucleus are larger than in any other grade. This decrease is due to the annulus now operating primarily in compression rather than tension due to its remodeling and accepting a larger portion of the compressive loads.

### Effect of Degeneration on Osmotic Pressure

The contours of the osmotic pressure are very similar to the initial fixed charge density profiles. This is due to the osmotic pressure being a function of initial and current fixed charge densities. The osmotic pressure in the central nucleus pulposus drops from approximately 0.4 to approximately 0.1 MPa. This explains the increasing inability of grades 3 through 5 to recover the fluid lost during the loading periods, since the osmotic pressure gradient is the primary mechanism with which fluid flows back into the disc.

These studies demonstrate the critical consequence of proteoglycan loss in the NP on the osmotic function of the NP tissue. Without wishing to be bound by any particular theory, the osmotic pressure of the material added should be such that the addition of the material increases the overall osmotic potential of the nucleus material. It is also desirable to have the osmotic pressure of the material be low enough that the resultant increase in pressure does not in itself cause pain. However, any increase in the osmotic pressure is also desirable. Therefore, a solution with an osmotic pressure above that of the native nucleus material is administered in to the degenerated disc.

### Example 2: Restoration of Proteoglycan to the Nucleus Pulposus of the Intervertebral Disc

The intervertebral disc is the largest avascular tissue in the human body and is mainly comprised of three different tissues. The central core, the nucleus pulposus, is surrounded by the outer annulus fibrosus and the upper and lower cartilaginous endplates. Lower back pain was reported in more than 80% of the cases exhibiting degeneration of lumbar intervertebral discs (Luoma et al., 2000, Spine 25(4):487). With aging, the proteoglycan and water content in the central nucleus reduces significantly, causing abnormal loading to the outer annulus (Urban et al., 1988, Spine (Phila Pa. 1976) 13(2):179-87; Luoma et al., 2000, Spine 25(4):487; Yerramalli et al., 2007, Biomechanics and Modeling in Mechanobiology 6(1):13-20; Urban et al., 2003, Arthritis Research and Therapy 5(3):120-38; Roughley et al., 2002, Biochemical Society Transactions. 30:869-74; Tropiano et al., 2005, The Journal of Bone and Joint Surgery 87(3):490-6; Olczyk, 1994, Z Rheumatol 53(1):19-25). In a dehydrated disc, the function of the nucleus, namely load transfer to the annulus through creation of an intradiscal pressure, is no longer occurring at a normal level. The mechanics of the degenerated disc are clearly altered compared to those of the intact disc (Yerramalli et al., 2007, Biomechanics and Modeling in Mechanobiology 6(1):13-20; Guerin et al., 2006, Journal of Biomechanics 39(8):1410-8; Boxberger et al., 2006, Journal of Orthopaedic Research. 24(9):1906-15)

An axisymmetric poroelastic model with incorporated osmotic swelling was utilized to model the stress distributions throughout IVDs of varying degenerative grades, including restoration to healthy levels. The interpolated fixed charge density (FCD) profiles were used to model changes in PG content of the IVD with degeneration.

An axisymmetric, poroelastic model was created using ABAQUS v6.5 finite element software (SIMULIA, Providence, R.I.). The model consists of a nucleus pulposus, an annulus fibrosus, cartilaginous and bony portions of the adjacent endplates, and cancellous and cortical portions of the corresponding vertebrae. The standard poroelastic theory included in ABAQUS is utilized, but a user-defined material was incorporated to include the effects of osmotic swelling. The model response was validated against experimental results such as axial displacement, radial displacement of the outer annulus fibrosus, and total fluid lost. Details of the creation of the model are described elsewhere herein.

Nucleus pulposus and annulus fibrosus tissue changes throughout the degenerative cascade. The material properties used include those to describe the solid portion, elastic modulus and Poisson's ratio; the fluid portion, void ratio and permeability; as well as the fixed charge density. Fixed charge density profiles were linearly interpolated from those of Urban, et. al, as shown in FIG. 3B. For each grade, material properties were altered to simulate degeneration of the intervertebral disc, according to Table 1. Most notably, degradation of the nucleus pulposus is believed to begin the degenerative process (which then causes the annulus fibrosus to degenerate, etc.), and therefore only the nucleus and not the annulus changes material properties from Grade 1 to Grade 2. Our proposed course of action for a degenerated disc is the replacement of the proteoglycans or any part thereof lost from the disc as degeneration occurs. In order to simulate this, the various grades of degeneration were modeled using the material properties shown in Table 1, with the exception of the fixed charge density profile, which was held constant at a Grade 1 level.

The results presented herein demonstrate that the stress profiles of varying grades of unaltered nucleus pulposus on the left side, and the equivalent grades with adjusted fixed charge density profiles on the right. For the unaltered conditions, Grades 2 and 4 have similar profiles, with a decrease in stress towards the outer nucleus seen in Grade 3. This is a result of the material properties assigned to each degenerative grade, as the annulus properties remain the same from Grade 1 to Grade 2 while the nucleus properties change. As the annulus stiffens in Grade 3, it accepts some of the additional stress from the nucleus.

Improving the fixed charge density profile decreases the stresses seen in the nucleus compared to the unaltered version, at each level. Grade 3 is nearly the same stress profile as the unaltered Grade 1, and by Grades 4 and 5, there is no discernible difference from a healthy Grade 1 condition.

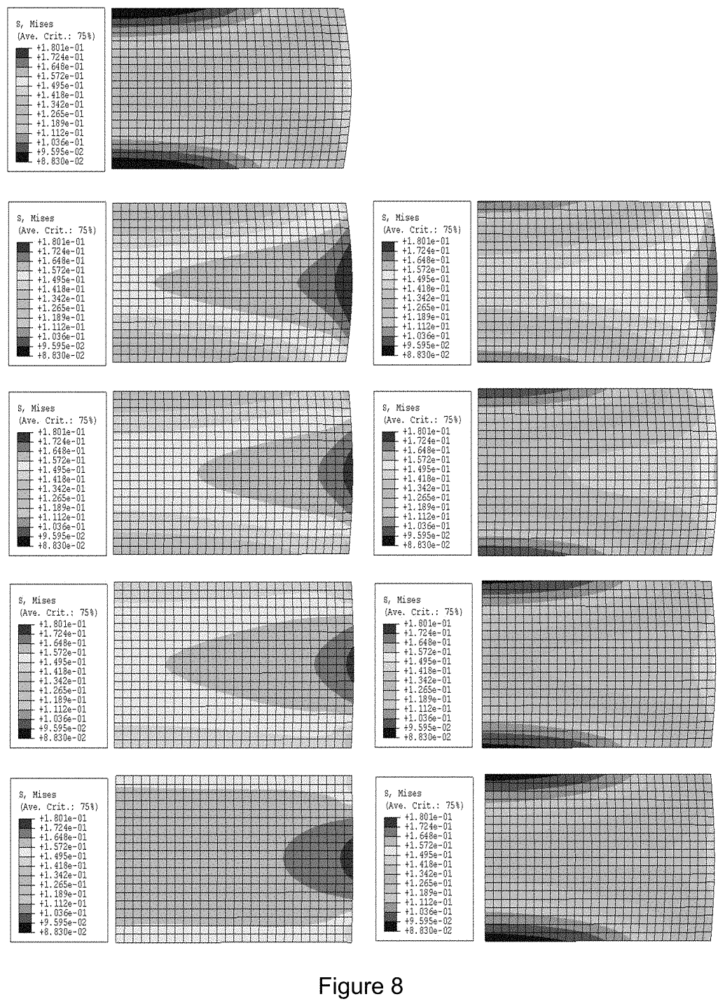

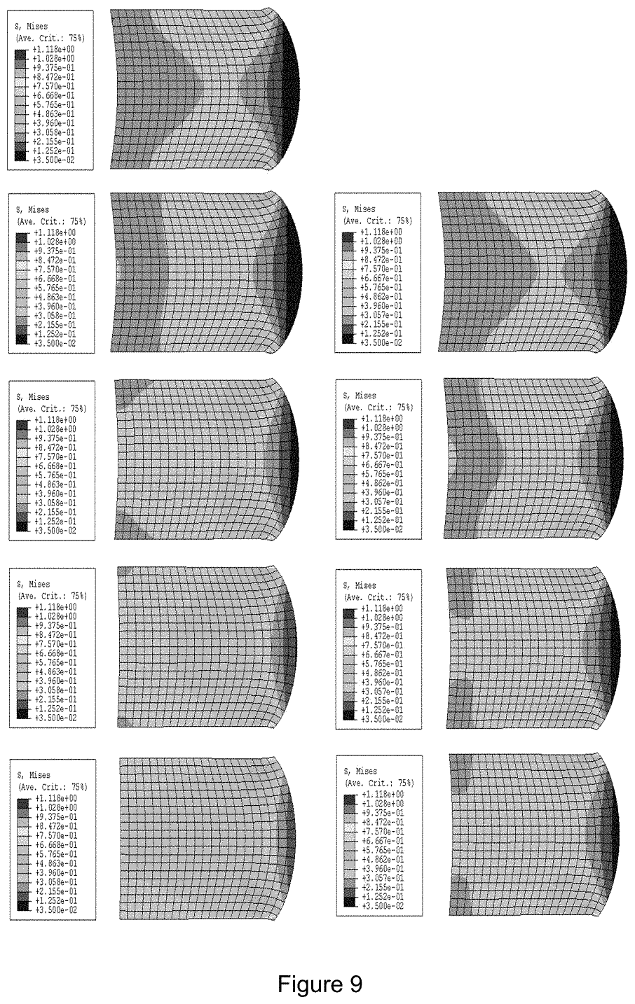

FIG. 9 shows the same relationships for the annulus fibrosus as those seen in FIG. 8 for the nucleus pulposus. The addition of proteoglycans to an otherwise degenerated disc decreases the stress in the annulus approximately one grade (e.g. Grade 2 with proteoglycan has a similar stress profile to the unaltered Grade 1, etc.), with the exception of Grade 5, which is nearly identical to the unaltered Grade 3, decreasing the stress by 2 grades.

When applying the Grade 1 fixed charge density profile to the degenerated discs of Grades 2 through 5, the stress experienced by the nucleus pulposus decreases dramatically, as shown in FIG. 8. Grades 3 through 5 are each practically returned to the stress profile seen in the unaltered Grade 1, and Grade 2 shows a substantial decrease from the unaltered Grade 2. Reductions in the stress carried by the nucleus pulposus tissue would likely slow down and perhaps stop completely the degenerative process in the nucleus. The greatest differences between the unaltered nucleus pulposus and that with proteoglycan added occur at Grades 4 and 5, implying that all levels of degeneration can benefit from this method of intervention.

A similar relationship exists for the annulus fibrosus as seen in FIG. 9, although not as steep of a drop in stress as seen in the nucleus pulposus. However, even a one-grade decrease in the stresses experienced by the annulus is a substantial improvement.

The results presented herein demonstrate that reverting the fixed charge density profile to its original “healthy” state decreases the stress experienced by the annulus fibrosus and drastically changes the stress on the nucleus pulposus, even though the other material properties are all still in a degenerated condition. These effects will lessen the need for the nucleus pulposus and annulus fibrosus to remodel to accommodate the new stresses experienced during degeneration, hence limiting the advancement of further degeneration.

In order to treat a degenerated disc by modulating the fixed charge density and thereby osmotic potential in the degenerated disc, aggrecan can be administered into the nucleus. The aggrecan must be large enough so as not to leave the disc space via diffusion or convective fluid flow. The aggrecan or any part thereof can be xenograft, allograft, or synthetic. Without wishing to be bound by any particular theory, the amount of aggrecan should be of a certain amount. The specific amount can be measured by disc pressure, disc height or volumetrically. The aggrecan can also be attached to a polymer backbone such as polyethylene glycol or polyvinyl alcohol or to a natural biomolecule such as HA, however this is not necessary. The backbone could also be used to administer components of aggrecan. The back bone provides additional structure to prevent aggrecan and/or components thereof from migrating out of the nucleus space.

Aggrecan and/or components thereof can be directly injected to the degenerated disc through a needle, preferably 18 gauge or thinner. The injection site is preferably at the posterior, lateral postiolateral and accomplished through a small cannula preferably 2 mm or less in diameter. This strategy offers distinct advantages over currently used steroid administrations by augmenting the structural mechanics of the disc. These administrations can easily be performed by an interventionist in a minimally invasive manner.

### Example 3: Nucleus Pulposus Augmentation

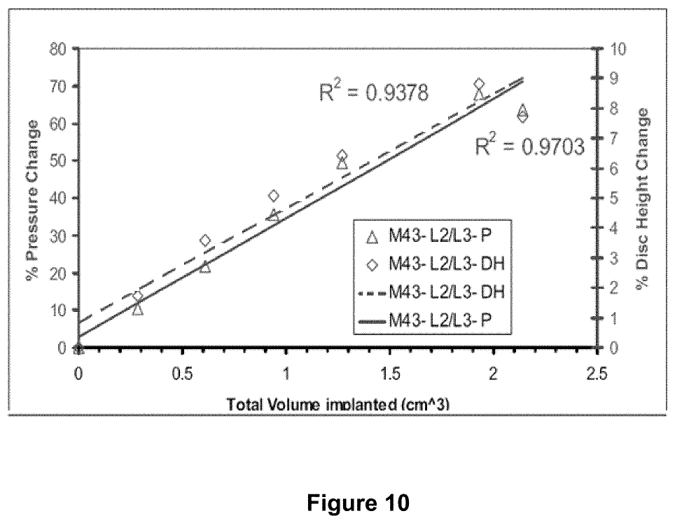

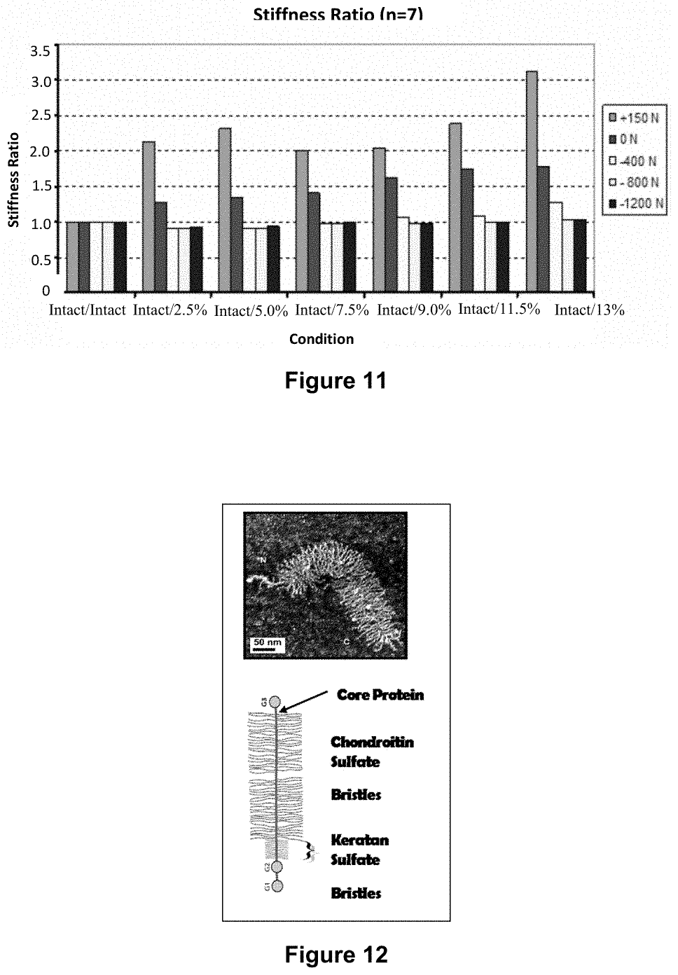

Prior work has investigated the role of the nucleus pulposus in human lumbar intervertebral disc mechanics. The nucleus is critical to the stability of the disc through the neutral zone (Joshi et al., 2008, J Biomech. 41(10):2014-111). After denucleation of the intervertebral disc, the neutral zone as well as the full range of motion was shown to increase significantly over the same measurements for the intact disc to which they were normalized. In addition, the stiffness of the disc through the neutral zone region was significantly reduced from that of the intact disc. This study shows that the nucleus is critical in providing stability to the intervertebral disc. In a separate study, the effect of inserting a hydrogel polymer into the nucleus cavity of an intact disc was investigated to determine the volume of material that can inserted and the resulting mechanical behavior of the augmented disc. It was shown that a linear relationship among volume of material inserted into the nucleus, change in intradiscal pressure and change in disc height. This relationship is interesting because it may allow a linear design guide to disc restoration through addition of a material to stabilize the disc. The work also showed that the stiffness of the disc through the neutral zone can be greatly enhanced by volume of material added in the augmentation. Augmentation or addition of volume of hydrogel material to the disc can alter biomechanics in a way that further stabilizes and stiffens the disc. Based on research on hydrogel polymers for augmentation of the intervertebral disc, it has been demonstrated that disc height and intradiscal pressure have a linear relationship to the volume of material administered into the nucleus (FIG. 10). These administrations result in an increase of stiffness of the disc and a reduction in the instability of the disc through the neutral zone (FIG. 11). Augmentation enables for restoration of disc biomechanics in a precise volume-controlled manner.

This work complements additional studies that have shown that nucleus removal and subsequent replacement with a hydrogel material will provide restoration back to the level of the intact disc (Arthur et al., 2010, Spine (Phila Pa. 1976) 35(11):1128-35). In a separate study, injections of CS and injections of a water control were made to a human cadaveric intervertebral disc. After cycling through a diurnal cycle, there was no difference in the CS disc from the water control or from the intact condition. This interesting study supports the findings by Ortiz et al (Seog et al., 2002, Macromolecules 35(14):5601-15; Han et al., 2007, Biophysical Journal 93(5):23-5; Han et al., 2007, Biophysical Journal 92(4):1384-98; Seog et al., 2005, Journal of Biomechanics 38(9):1789-97; Dean et al., 2006, Journal of Biomechanics 39(14):2555-65; Ng et al., 2003, Journal of Structural Biology 143(3):242-57; Buschmann et al., 1995, J Biomech Eng. 117(2):179-92; Dean et al., 2003, Langmuir 19(13):5526-39) that mechanical stability is controlled not only by hydration (obtained with CS), but by electrostatic interactions resulting from macromolecular architecture. These studies led to the strategy of mimicking the macromolecular architecture of aggrecan that not only hydrates, but that provides electrostatic repulsion equivalent to that to natural aggrecan.

### Example 4: Enzymatically Resistant Biomimetic Aggrecan as an Augmentation Material

To stabilize the disc early in the degenerative cascade, an injection to the nucleus pulposus, or inner region of the disc was designed to enhance the osmotic and hydration potential of the tissue while also serving to enhance the intradiscal pressure, thus “re-inflating the flat tire”. This approach is also intended to mechanically protect the annulus fibrosus from abnormally high stresses which may be responsible for the formation of tears and fissures. One approach to mechanically stabilizing the disc is by increasing the main disc proteoglycan, aggrecan, concentration in the nucleus pulposus back to a normal level.

While administrations of aggrecan may be useful, the cost of the material at this point in time is prohibitive for any type of realistic intervention (Sigma). In addition, while injections of natural aggrecan may be useful, commercially available aggrecan would be subject to the same limitations as the body's own aggrecan, enzymatic degradation of the protein core, which fragments the molecule and allows for migration of the fragments by convective diffusion from the intradiscal space, further reducing the hydration and mechanical stability of the intervertebral disc (Raj et al., 2008, Pain Pract 8(1):18-44; Urban et al., 2004, Spine (Phila Pa. 1976) 29(23):2700-9).

### Aggrecan Structure

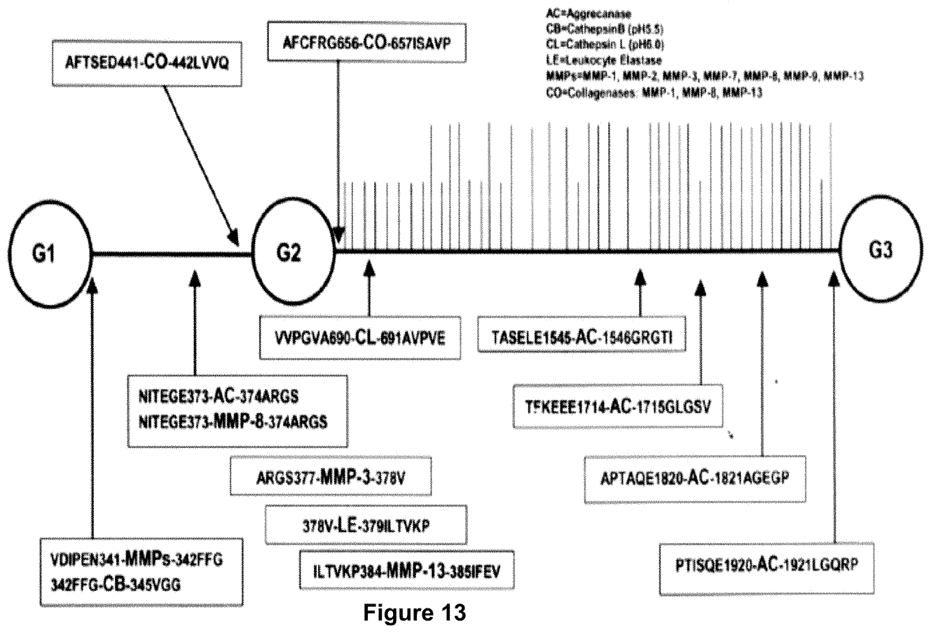

Aggrecan is a three-dimensional molecule that includes a protein core from which bristles of gylcosaminoglycans (chondroitin sulfate and keratan sulfate) radiate in all directions, forming a “bottle-brush” structure (FIG. 12). The molecule functions on two levels: 1) it allows water uptake by the nucleus due to sulfated groups in the chondroitin and keratan sulfate regions which, in part, provide intradiscal pressure and 2) it provides electrostatic repulsion due to the 3D macromolecular structure, which contributes to intradiscal pressure and disc height.

Aggrecan, an aggregating proteoglycan, is the major proteoglycan of the intervertebral disc. Aggrecan consists of a protein core approximately 300 kDa and 400 nm in contour length (Nap et al., 2008, Biophysical Journal 95(10):4570-83). The protein core consists of several domains which allow for the molecules flexibility (IGD) attachment to hyaluronic acid (G1 globular domain) attachment of chondroitin sulfate (CS) (CS1 and CS2 domains) and keratan sulfate (KS) (KS domain) and cell signaling (G3 globular domain). Approximately 100 CS glycosaminoglycan (GAG) chains are covalently attached to the core protein in the CS region with a grafting density of approximately 0.25 to 0.5 nm¹. Each CS chain which consists of 10-50 repeating disaccharide units of glucuronic acid (GlcUA) and n-acetylgalactosamine (GalNAc) and is approximately 20 kDa with a length of 40 nm (Muir, 1977, Ann Rheum Dis. 36:199-208). The KS region of the aggrecan core protein is smaller with only ˜30 KS chains attached. KS is a smaller GAG chain of 5-15 kDa. Approximately 8000-10000 negatively charged groups are present in the aggrecan bottle brush via the charged sulfate and carboxylic acids of the attached CS and KS chains (85-86). Although aggrecan is able to associate with HA and link protein extracellularly, large HA-aggrecan aggregates are only predominant in infancy such that by 6 months of age only approximately 30% of the NP is in the aggregate form and levels as low as 10% aggregation are seen in the adult NP (87-88).

### Enzymatic Degradation of Aggrecan and Other Proteoglycans

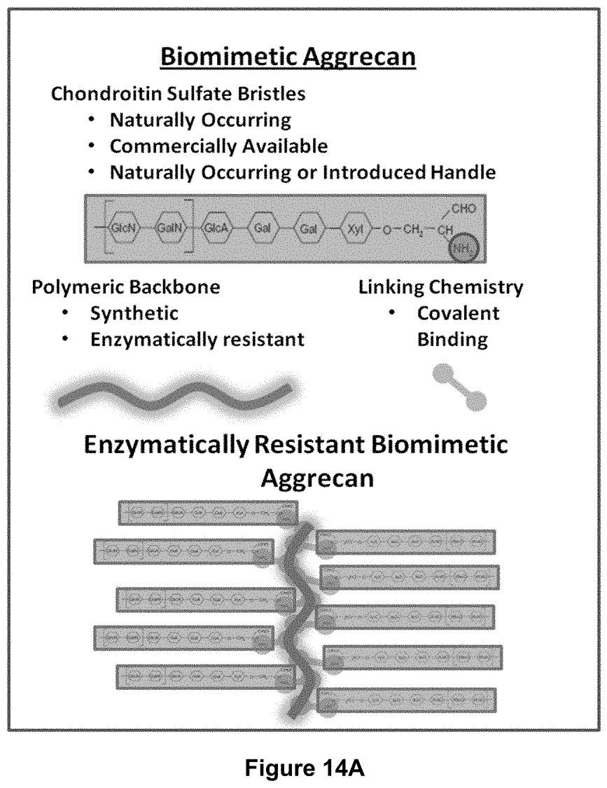

Enzymatic degradation of aggrecan and other proteoglycans in vivo allow for the turnover of matrix material (Kiani et al., 2002, Cell Research 12(1):19-32). However, in the IVD where nutrition is limited, NP cells are in a state of senescence and are unable to produce aggrecan at the necessary rates to maintain normal aggrecan concentration (Roberts et al., 2006, European Spine Journal 15:312-6; Zhao et al., 2007, Ageing Research Reviews 6(3):247-61). Enzymatic activity in the disc increases with aging and degeneration, resulting in the presence of smaller aggrecan fragments and the loss of overall aggrecan concentration (Patel et al., 2007, Spine 32(23):2596-603). Enzymatic cleavage of aggrecan is targeted to the core protein of the molecule and does not affect the CS region (Kiani et al., 2002, Cell Research 12(1):19-32). Matrix metalloprotinases (MMP) and aggrecanases are the main enzymes that contribute to the degradation of aggrecan. In particular MMPs 1, 3, 7, 9, and 13 have showed increased activity with degeneration as well as aggrecanase 1, 4, 9, 5, and 15 (Roberts et al., 2000, Spine 25(23):3005-13; Goupille et al., 1998, Spine (Phila Pa. 1976) 23(14):1612-26; Le Maitre et al., 2007, Biochemical Society Transactions 35:652-5). Several cleavage points for these (and other) enzymes exist throughout the aggrecan core protein resulting in varying sized fragments of aggrecan. The aggrecan fragments vary in functional capacity, such as electrostatic repulsion and osmotic potential, as well as the increased tendency to migrate out of the nucleus pulposus through the endplates, related to the size of the fragments. (FIG. 13).

Transport through the disc endplates largely governs the disc environment. Studies into the transport properties of cartilaginous endplates revealed a dependence on molecule size, conformation (globular or long chain) and charge and can affect a molecules ability to diffuse through the endplate. Smaller molecules (i.e. 100d) can leave the matrix to a greater extent than larger ones (i.e. 10 kd). Long chain conformations (i.e. different MW PEG chains were investigated) are more restricted from leaving the matrix than globular conformations (i.e. starch). Therefore, the enzymatic degradation of the aggrecan molecule into smaller less structured fragments may limit the longer term benefits of a native aggrecan replacement.

### Synthetic Bottle Brush Polymers and Less-Ordered Hybrid Biomacromolecules

The synthesis of synthetic-based bottle brush polymers or “molecular bottle brushes” has been extensively studied and reviewed (Sheiko et al., 2008, Progress in Polymer Science 33(7):759-85; Zhang et al., 2005, Journal Of Polymer Science Part A Polymer Chemistry 43(16):3461-3481; Gao et al, 2007, Journal of the American Chemical Society 129(20):6633-9). The three main synthetic methods are grafting-to, grafting-through and grafting-from. In grafting-to, bottle brush bristles in the form of a monotelechelic polymer is attached to a functionalized polymeric core (Gao et al., 2007, Journal of the American Chemical Society 129(20):6633-9). In the grafting-through strategy, a macromonomer is combined with initiator in order to induce polymerization of the polymerizable end of the macromonomer building the polymeric core as the macromonomers are joined together, often via free-radical polymerization (Ito, 1998, Progress in Polymer Science 23(4):581-620). In a third strategy, grafting-from, a macroinitiator polymeric core is combined with monomer which is subsequently polymerized off of the core via initiation and propagation of the free-radical generated by the initiator. Each of these strategies has advantages and disadvantages in terms of grafting density of side chains, or “bristles,” on the core, degree of polymerization of side chains and degree of polymerization of the core. In addition to densely packed brushes, sparse brushes, stiff brushes, flexible brushes, multi grafted brushes, gradient brushes, stars and networks may also be formed. The Ortiz group, for example (Zhang et al., 2005, Macromolecules 38(6):2535-9; Zhang et al., 2004, Macromolecules 37(11):4271-82; Zhang et al., 2005, Macromolecules 38(6):2530-4), fabricated a family of end functionalized polymer brushes of poly(2-hydroxyethyl methacrylate-g-ethylene glycol) with varying polymeric core length, and brush grafting density and demonstrated mechanical properties in the presence of various stimuli. Synthetic glycopolymer brushes have been fabricated with short monosaccharide or oligosaccharide side chains which impart biological function to the polymers, however the short bristle length (compared to CS) inhibited the mechanical function of the molecules (Ladmiral et al., 2004, European Polymer Journal 40(3):431-49; Okada, 2001, Progress in Polymer Science 26(1):67-104; Lutz et al., 2008, Progress in Polymer Science 33(1):1-39). Attempts have also been made for the fabrication of proteoglycan-like cylindrical glycopolymer brushes (Muthukrishnan et al., 2005, Macromolecules 38(19):7926-34) as well as brushes with charged sulfonate bristles (Lienkamp et al., 2006, Macromolecular Chemistry and Physics 207(22):2066-73). These brush structures emulate the architecture of the aggrecan brush structure. However, the fully synthetic systems were not able to mimic the biological activity of the natural biomolecule.

In a separate body of work, the strategy of replacing CS directly has been investigated in copolymers that have been blended or cross-linked into interpenetrating networks with less order than the bottle-brush configuration. Solutions of CS have been mixed with HA and the rheological properties of the solutions have been shown to be improved slightly with the addition of CS, but to a lesser extent than if aggrecan is utilized in place of CS (Nishimura et al., 1998, Biochim Biophys Acta 1380(1):1-9). Crosslinkable CS has also been investigated by the Elisseeff group (Li et al., 2004, Journal of Biomedical Materials Research 68(1):28-33) where methacrylated CS macromers are generated by modifying hydroxyl groups along the CS backbone allowing for subsequent photopolymerization. Hydrogels from the methacrylated CS were polymerized and their mechanical properties investigated. In this method, the CS chains remain disordered achieving only part of their mechanical potential via osmotic swelling properties.

These polymers and materials exhibited good hydration, however, they do not provide structural function because the geometrical arrangement of polymer chains is relatively unorganized (especially in comparison to the highly ordered brush structure described here). Prior to the present invention, natural CS had not been synthesized into a bottle brush polymeric but was deficient in many aspects including susceptibility to enzymatic degradation. The present invention relates to a biomimetic approach that models the macromolecular geometry of aggrecan while limiting enzymatic degradation.

In addition to being resistant to enzymatic degradation, the biomimetic aggrecan of the invention also exhibits multifunctional properties such as regulating osmotic pressure and have desirable mechanical strength.

### Mechanical Role of Aggrecan

Aggrecan has two main mechanical functions in the disc: 1) it allows water uptake by the nucleus due to sulfated groups in the chondroitin and keratan sulfate rich regions which, in part, provides intradiscal pressure (Elliott et al., 2001 Journal of Biomechanical Engineering 3:256-63) and 2) it provides electrostatic repulsion due to the elegant 3D macromolecular bottle brush structure, which contributes to intradiscal pressure and disc height (Wilke et al., 1999 Spine 8:755-62). A main constituent of aggrecan is the GAG chains which are covalently attached to the aggrecan protein core and comprise approximately 80% of the total weight of the molecule. These GAG chains, in particular in the CS rich region are arranged in closely packed arrays creating a bottle brush structure. Along with the hydrating properties of the GAG chains imparted by the charged groups along the molecule, electrostatic forces between closely packed GAG chains provide a mechanical resistance to applied force. Electrostatic forces between CS chains account for 50% (290 kPa) of the equilibrium compressive elastic modulus as predicted by theoretical modeling (Johannessen et al., 2004 Annals of Biomedical Engineering 1:70-6). These electrostatic repulsion forces however will only occur when intermolecular distances are ˜5 Debye lengths or less (CS concentration of 30 mg/mL, 2-4 nm spacing between chains) (Roughley et al., 2004 Spine 23:2691-9). Interactions between opposing GAG chains have been experimentally demonstrated to resist force at short distances, however, when opposing aggrecan chains are brought into contact, they exhibit enhanced force resistance at larger distances demonstrating the benefit of the more ordered arrangement of CS chains seen in the aggrecan bottle brush (Urban et al., 1980 2:63-9).

### Example 5: General Strategy for Biomimetic Aggrecan

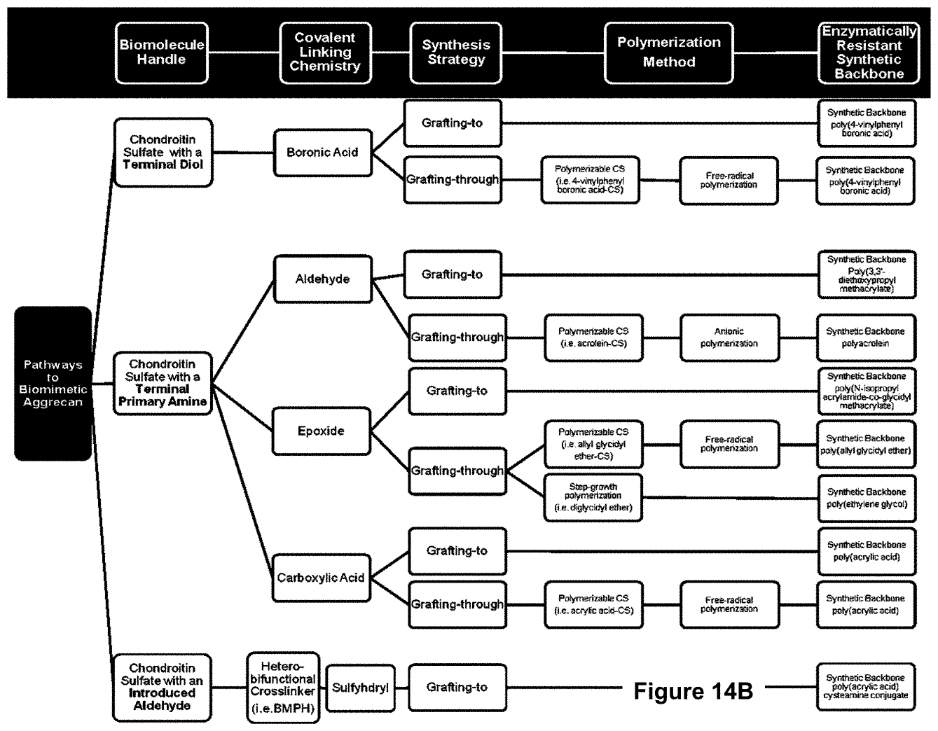

The strategy for the restoration of GAG content, and thus FCD to the NP, includes developing a biomimetic aggrecan molecule with chondroitin sulfate (CS) molecules attached to a synthetic core structure in a bottle brush form. It is important that the CS be organized in this way because the distance between adjacent CS molecules effects their physical resistance of force. Electrostatic forces between CS chains account for 50% (290 kPa) of the equilibrium compressive elastic modulus as predicted by theoretical modeling (Buschmann et al., 1995, J Biomech Eng. 117(2):179-92; Eisenberg et al., 2005, Journal of Orthopaedic Research 3(2):148-59). These electrostatic repulsion forces however will only occur when intermolecular distance between CS chains is 2-4 nm (Seog et al., 2002, Macromolecules 35(14):5601-15). In addition, the immobilization of CS into a larger structure is imperative in maintaining residence time of these molecules in the tissue where CS generally has a MW between 15-50K daltons while aggrecan has a MW of approximately 2,000K daltons. It is also important to utilize a synthetic polymeric core in order to resist enzymatic degradation where enzymatic activity is targeted to protein moieties. CS molecules are attached to a polymeric backbone via interactions of a functional handle at the terminal end of CS and a covalent linkage to a preformed (“grafting-to strategy”) or concurrently built (“grafting-through” strategy) polymeric backbone (FIG. 14). It is important to note, both synthetic strategies utilize known polymerization and modification chemistries to create a hereto unknown polymer brush. Utilizing this strategy, the synthesis of several different biomimetic aggrecans is feasible (FIG. 14). Different handles on the chondroitin sulfate may be utilized including a terminal diol, a terminal primary amine or an introduced aldehyde group. These handles may then be covalently bound to a synthetic component via several different linking chemistries including boronic acid, aldehyde, epoxide, carboxylic acid and sulfhydryl interactions. The biomimetic aggrecan may then be polymerized into a bottle brush structure via the “grafting-to” or “grafting-through” polymerization strategies. The resulting structure consists of natural chondroitin sulfate bristles and one of several possible polymeric backbones as demonstrated in FIG. 14.

### Example 6: Synthesis of Biomimetic Aggrecan Via the Terminal Diol-Boronic Acid Linking Chemistry

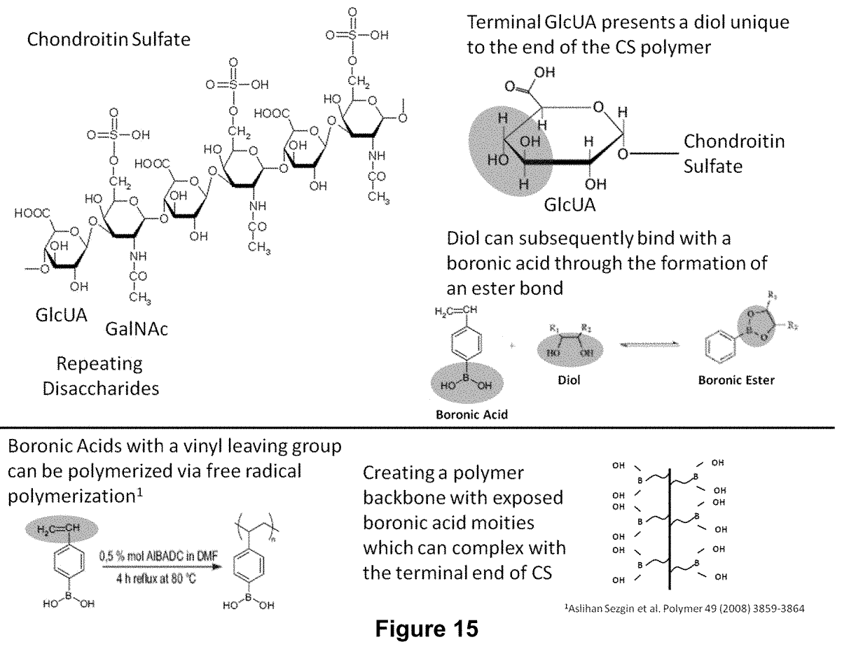

Utilizing the high affinity complexation of boronic acids with compounds containing diols, a novel polymer system via free radical polymerization techniques which consists of a boronic acid functionalized polymer core to which three-dimensional brush “bristles” of chondroitin sulfate is attached (which mimics the bristles of the aggrecan molecule) has been developed (FIG. 15). This unique structure enables rehydration of the disc and restoration of the intradiscal pressure, which in turn restores the disc stability and biomechanical behavior to that of a healthy disc.

The boronic acid in the invention serves as a linker between a polymer of specific characteristics and the terminal end of CS. The biomimetic aggrecan of the invention has a brush structure that mimics aggrecan and therefore is able to draw in and hold water. The invention relates to using selective attachment of a boronic acid to only the terminal diol of CS which is structurally different and differently accessible from the other diols presented throughout the macromolecule. This can be achieved by modifying the phenylboronic acid (PBA) moieties through the incorporation of domains into the polymer to selectively target particular diol configurations or by increasing the ratio of CS to boronic acid thereby resulting in a more brush like structure. In some instances, the biomimetic aggrecan of the invention can be achieved by adding an appropriate excess of CS to a boronic acid containing polymer thereby forming a polymer brush. By selectively attaching CS terminal groups to a polymeric backbone via the boronic acid interaction, ordered structures can designed to have a particular shape including brush (densely grafted CS), comb (sparsely grafted CS) and dendritic (CS grafted to branched boronic acid polymers). Such different configurations created can have different functional outcomes.

### Example 7: Synthesis of Biomimetic Aggrecan Via the Terminal Primary Amine Handle

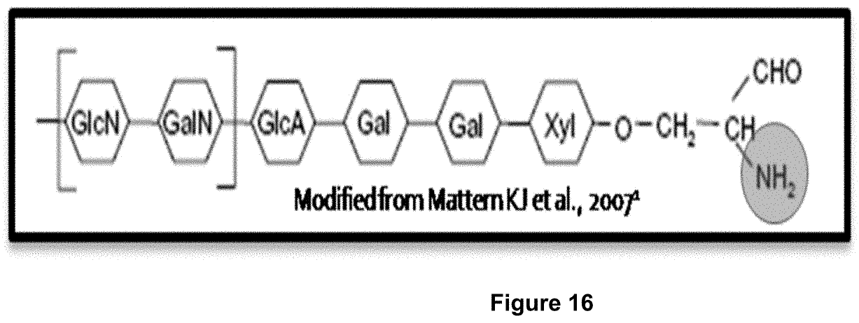

The fabrication of a bottle brush polymer with natural chondroitin sulfate side chains requires the terminal-end immobilization of CS. Commercially available natural CS was investigated for a terminal primary amine (PA) that may be present as a result of CS isolation from donor tissues (FIG. 16) (Anderson et al., 1965, Journal of Biological Chemistry 240(1):156-67; Mattern et al., 2007, Carbohydrate Research 342(15):2192-20). The following experiments were designed to investigate the presence of PAs in CS from various suppliers as isolation techniques differ from vendor to vendor and based on CS type and source.

CS was investigated from two vendors (Calbiochem and Sigma) and three tissue sources (bovine trachea, bovine cartilage and shark cartilage). The fluorescamine assay, which is sensitive to primary amines was used to detect PAs in the CS macromolecule (Udenfriend et al., 1972, Science 178:871-2). Fluorescamine, a fluorometric reagent, reacts directly with primary amines to yield highly fluorescent derivatives (390 nm excitation, 475 nm emission) whose resulting fluorescence is proportional to the amine concentration (Udenfriend et al., 1972, Science 178:871-2). CS was solubilized in sodium borate buffer (SBB, pH 9.4) at various concentrations (10 mg/mL to 0.01 mg/mL). 150 μl samples of CS solution were added to a 96-well plate then incubated with 50 μl of 3 mg/mL fluorescamine solution (solubalized in DMSO) for 5 min.

Sample fluorescence was measured on an Infinite M200 TECAN spectrophotometer with excitation/emission of 365/490 nm Fluorescence was normalized to SBB blanks and samples were taken in triplicate. L-serine (MW, 105.09) which is the attachment site for CS to the aggrecan core protein, and contains only one PA per molecule, was used to establish a fluorescence vs. [PA] standard curve. The number of PA/molecule of CS was calculated from the linear region of the L-serine standard curve (Table 2). CS-4 from sigma (Sigma C6737) was found to have ˜1 PA/CS chain making it ideal for use in the synthesis of our biomimetic aggrecan structures. All other CS tested showed a higher PA content which may arise from protein impurities or over processing of CS during isolation.

TABLE 2
Primary Amine Content of CS for Varying Sources

| | Tissue | | Estimated | #PA/CS |
|---|---|---|---|---|
| Product | Source | CS Type | MW⁽⁵⁻⁶⁾ | Chain |
| Sigma | Shark | Primarily CS-6 | ~65,000 | 9.91 +/− 0.74 |
| C4384 | Cartilage | | | |
| Sigma | Bovine | 60% CS-4, | ~22,000 | 2.9 +/− 0.24 |
| C9819 | Trachea | 40% CS-6 | | |
| Sigma | Bovine | Primarily CS-4 | ~22,000 | 1.06 +/− 0.07 |
| C6737 | Cartilage | | | |
| Calbiochem | Bovine | Mix of CS-4, | ~22,000 | 6.78 +/− 0.53 |
| 230699 | Trachea | CS-6, CS-4,6, | | |
| | | CS-2,6 | | |

For all further studies, CS-4 from Sigma can be utilized however any CS with one primary amine per molecule may be used. In general, CS-4 may also be beneficial over CS-6 as it is the more abundant CS in young NP tissue and is derived from a mammalian cartilage source. CS-4 has also been widely used in therapeutic settings with demonstrated anti-inflammatory and anti-oxidant effects (Lauder, 2009, Complementary Therapies in Medicine 17(1):56-62).

Several amine reactive chemistries were investigated for their reactivity to the CS terminal primary amine. The monomers acrolein, allyl glycidyl ether (AGE) and acrylic acid were purchased from Sigma Aldrich. Acrolein contains an amine reactive aldehyde functionality where upon reaction of an aldehyde with a primary amine in alkaline conditions (pH greater than 9.0), an imide bond will form (Hermanson G T. Bioconjugate Techniques. Second ed. Pierce Biotechnology TFS, editor. Rockford, Ill., USA: Academic Press; 2008). AGE, is an epoxide containing monomer, which will react with amines also at alkaline pH via the opening of its oxirane ring (116). Acrylic acid is a monomer with carboxylic acid functional groups. The carboxylic acid group of acrylic acid can be activated to be highly reactive with amines using well characterized EDC/sulfo-NHS coupling reactions (Hermanson G T. Bioconjugate Techniques. Second ed. Pierce Biotechnology TFS, editor. Rockford, Ill., USA: Academic Press; 2008).

For the conjugation of CS to acrolein and AGE, solutions of monomer at various concentrations in 0.1M SBB, pH 9.4, were mixed with solutions of CS (11 mg/mL) in 0.1M SBB pH 9.4 to achieve varying monomer:CS molar ratios. For acrolein-CS samples, sodium cyanoborohydride (5M in 1N NaOH, Sigma) was added at 20 μL/mL in order to stabilize the formed Schiff base to a secondary amine bond (Hermanson G T. Bioconjugate Techniques. Second ed. Pierce Biotechnology TFS, editor. Rockford, Ill., USA: Academic Press; 2008). Acrylic acid was first activated with EDC/sulfo-NHS (2 mM EDC and 5 mM sulfo-NHS in MES buffer (0.05M MES, 0.5M NaCl), pH 6.0) for 15 min followed by quenching of excess EDC with 2-mercaptoethanol (10 min at a final concentration 20 mM). Activated acrylic acid was then combined with CS (CS in phosphate buffered solution, pH 7.5) at varying monomer:CS molar ratios. All monomer-CS solutions were placed on a rotator and allowed to react for 4 hrs. CS-Acrylic acid solutions were then filtered using a sephadex G-25 pre-packed desalting column (PD-10, GE Healthcare) to remove excess reactants. 150 ul samples of each solution were taken in triplicate and assayed with the fluorescamine assay as described previously for their PA content. The percentage of PAs in the CS sample conjugated to monomer is indicated by the percent decrease in PA content which was calculated as

$$[ \text{?} ] * 100 \%$$

$$\text{?} \text{indicates text missing or illegible when filed}$$

A decrease in the PA content of the CS-monomer solutions with respect to CS without monomer is indicative of binding of the monomer to CS at the primary amine. Solutions of monomer without CS were also assayed with the fluorescamine reagent and found to exhibit no appreciable fluorescent signal at the excitation/emission of interest.

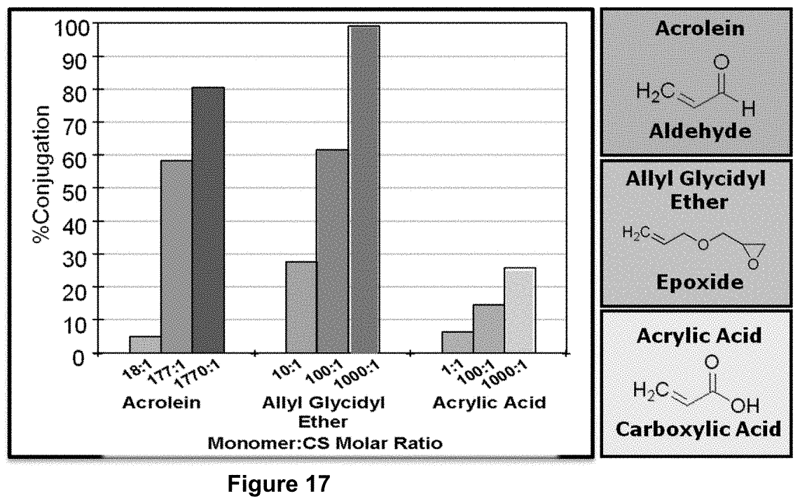

As the molar ratios of monomer:CS was increased, an increase in the % Conjugation was observed for all monomers (FIG. 17). In all cases, % conjugation was modulated by monomer:CS molar ratio with the maximum conjugation seen at a molar excess of monomer at 1000:1. A large molar excess is likely required due to the small concentration of PAs available in comparison to CS concentration. Acrylic acid:CS conjugation only reached a maximum of 26% at a 1000:1 molar ratio. This may be due in part to the several steps required to activate acrylic acid for reaction with PAs. Almost full conversion of PAs was seen with AGE (99%) at a molar ratio of 1000:1 AGE:CS indicating the epoxide-amine reaction as the most facile for attachment of CS by its terminal end.

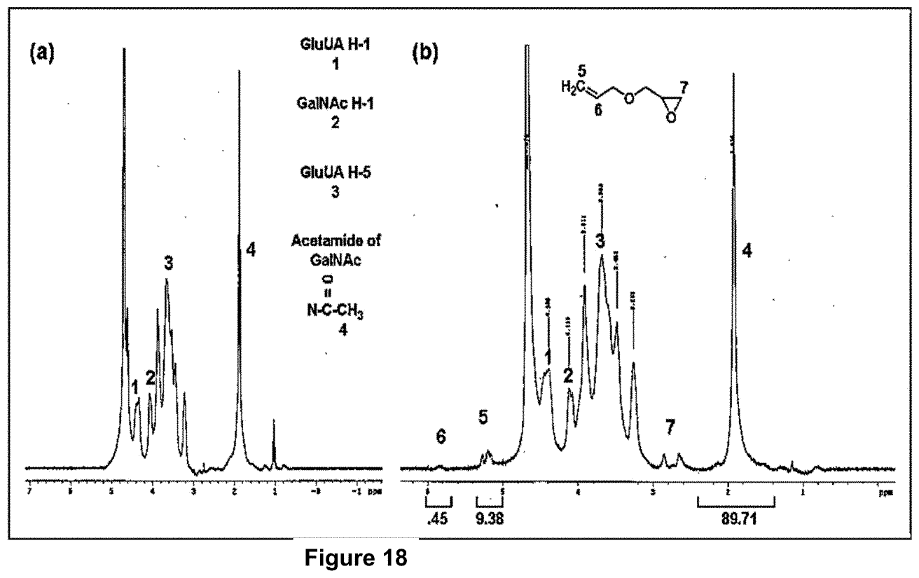

The conjugation of AGE monomer to CS was further investigated using ¹H-NMR. CS-AGE conjugate samples were prepared as described previously followed by gravity column filtration in a pre-packed Sephadex G-25 M column (PD-10, GE Healthcare) in order to remove un-reacted monomer. Filtered sample was lyophilized overnight then re-constituted in D₂O at approximately 30 mg/mL. ¹H-NMR spectra were taken on a 300 MHz NMR spectrometer (UNITYNOVA) at 64 scans and at ambient temperature (FIG. 18). Spectra were acquired for both CS and CS-AGE conjugates. Several classes of protons were resolved and assigned on the basis of their chemical shifts for CS and AGE monomer (Toida et al., 1994, Analytical Sciences 10(4):537-41; Toida et al., 1993, Analytical Sciences 9(1):53-8).

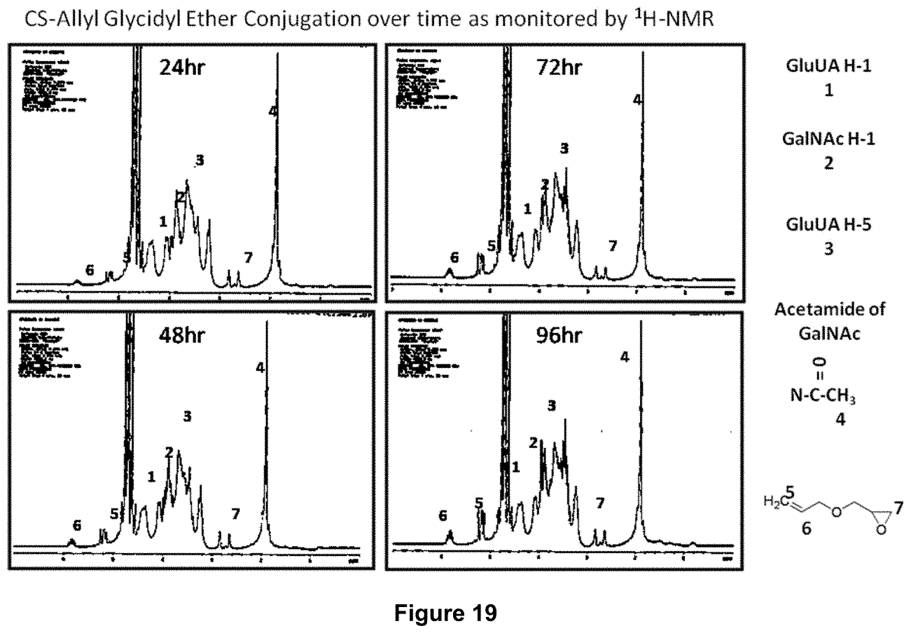

CS spectra after conjugation (FIG. 18b) matched well with CS spectra before conjugation (FIG. 18a) and reported literature ¹H-NMR spectra for CS-4, indicating no major modification of the CS main chain with our reaction technique. Appearance of AGE associated peaks (peaks 6, 5, and 7 in FIG. 18b) indicate presence of the AGE monomer however the signal is low in comparison CS. A much higher AGE:CS ratio in the ¹H-NMR spectra would be expected if side reactions of the monomer were occurring within the CS disaccharide or if a large excess of free monomer was present in the sample solution. This is an important distinction as epoxides are reactive to several other functional groups including carboxylic acids and hydroxyls (both present in CS) however at moderately basic pH (pH between 9 and 11) epoxide-amine reactions are favorable (Hermanson G T. Bioconjugate Techniques. Second ed. Pierce Biotechnology TFS, editor. Rockford, Ill., USA: Academic Press; 2008). When CS was allowed to react with AGE over longer periods of time (up to 96 hrs) the AGE content of the CS increased as indicated by ¹H-NMR and the CS signal became disrupted indicating possible side reactions of the AGE epoxide with other functional groups of the CS such as hydroxyls or carboxylic acids (FIG. 19). In order to synthesize a CS monomer with only one vinyl group attached via the allyl glycidyl ether the reaction time between CS and AGE must be limited.

The next set of experiments was designed to utilize the CS terminal end primary amine handle in the “grafting-to” strategy of synthesis.

In order to further investigate the utility of the CS terminal primary amine-epoxy interaction in the immobilization of CS using the grafting-to strategy, CS attachment to epoxide-functionalized glass surfaces was monitored by measuring surface hydrophilicity. Conjugation of CS to epoxide-functionalized glass slides was conducted in solution at a pH of 9.4 at room temperature.

Surface hydrophilicity was measured using a contact angle meter with DI water as the medium. A change in contact angle is expected as CS is deposited onto the substrates since CS is a charged molecule and will attract water making the surface more hydrophilic, thereby decreasing the measured contact angle. Functionalized glass slides as well as un-functionalized glass slides were soaked in 3 mL of CS solution in sodium borate buffer (SBB, pH 9.4, 4 hr) at varying CS concentration. Slides were subsequently rinsed thoroughly with fresh SBB to remove any non-covalently bound CS. Un-functionalized glass slides were similarly prepared as control samples.

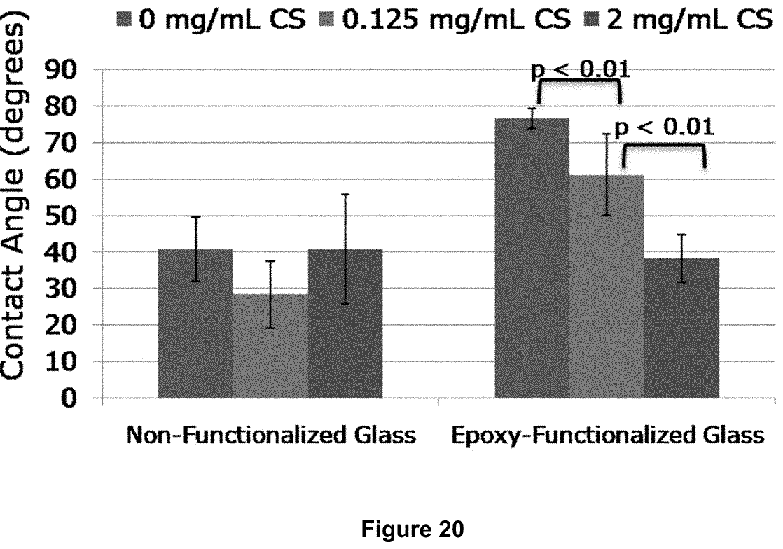

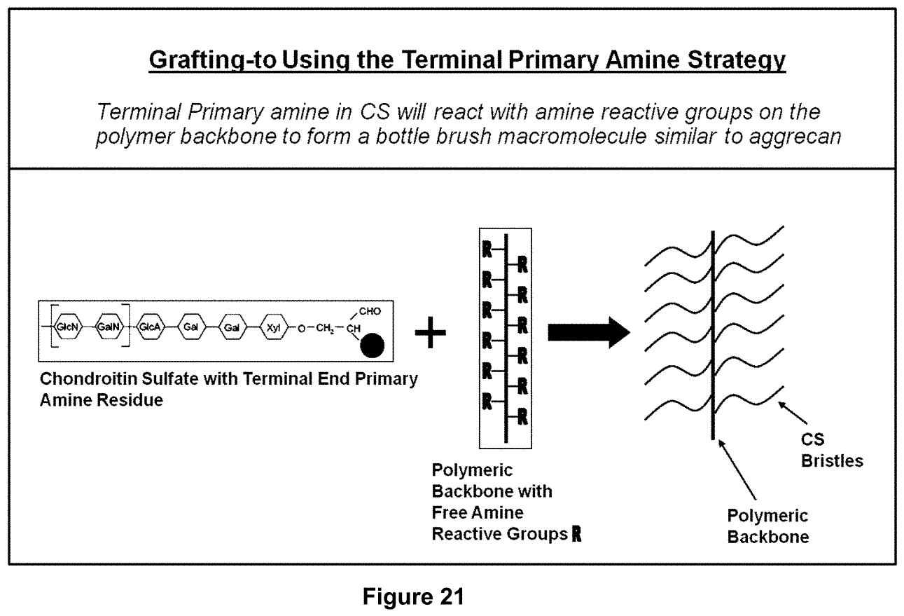

On epoxy-functionalized slides, contact angle without CS was measured at 76.5±2.8°. With the addition of CS at 0.125 mg/ml contact angle was reduced to 61.1±11.2° and further reduced to 38.2±6.2° with the addition of CS at 2 mg/ml indicating an increase in hydrophilicity associated with the deposition of charged CS on the epoxy-functionalized surface (FIG. 20). These surface studies provide evidence that CS can be immobilized onto amine reactive substrates via the terminal primary amine of the CS chain. Such immobilization can be transferred to polymeric chains for the synthesis of CS bottle brush polymers via the “grafting-to” method (FIG. 21).

The results presented herein indicate that possible linking chemistries include but are not limited to aldehyde, epoxide and carboxylic acid chemistries. Utilizing the grafting-to strategy, possible polymeric backbones for biomimetic aggrecan include but are not limited to Poly(3,3′-diethoxypropyl methacrylate) (Hwang et al., 2007, Journal of Controlled Release 122(3):279-86) which utilizes the aldehyde linking chemistry, poly(N-isopropyl acrylamide-co-glycidyl methacrylate) (Nguyen et al., 1989, Biotechnology and Bioengineering 34(9):1186-90) which utilizes the epoxide linking chemistry and poly(acrylic acid) (PAA) which utilizes the carboxylic acid linking chemistry. The synthesis of a biomimetic aggrecan with a PAA polymeric backbone via the grafting-to strategy will be further discussed as an example of this strategy.

### Example 8: Synthesis of PAA-Based Biomimetic Aggrecan Via the Grafting-to Strategy Utilizing the Terminal Primary Amine Handle

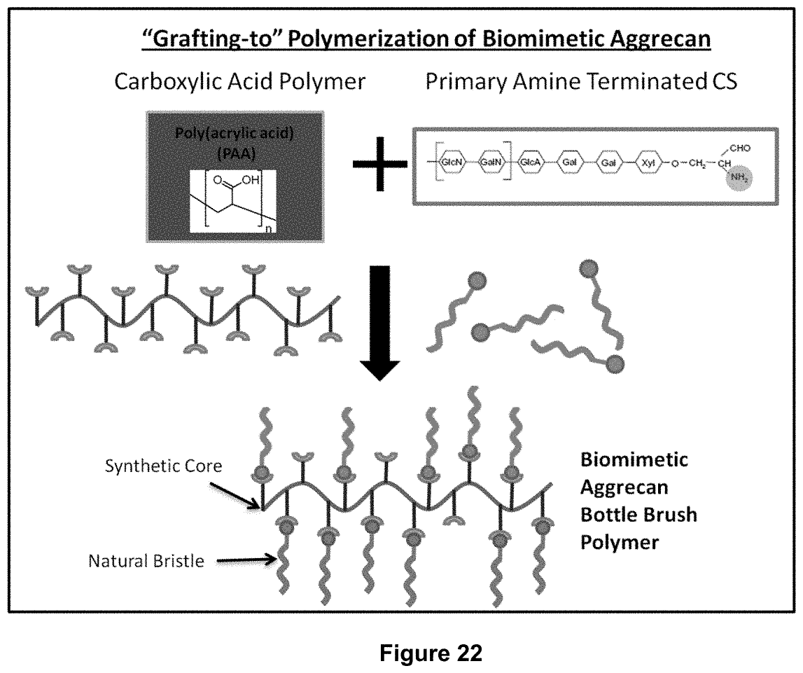

The polymeric backbone of poly(acrylic acid) (PAA) was chosen as an example of the synthesis of CS-glycopolymer structures via the “grafting-to” strategy. PAA of 250 kDa MW was purchased from Sigma in order to mimic the MW of the natural aggrecan protein backbone. PAA is a linear polymer with an enzymatically resistant hydrocarbon backbone and pendant carboxylic acid groups (FIG. 22). PAA has been used in the hydrogel form with bioactive molecules for the culture of cells and is shown to be non-toxic in in vitro studies (Mattern et al., 2007, Carbohydrate Research 342(15):2192-201). The carboxylic acids of PAA can be activated via reaction with EDC/sulfo-NHS for further reaction with primary amines (Hermanson G T. Bioconjugate Techniques. Second ed. Pierce Biotechnology TFS, editor. Rockford, Ill., USA: Academic Press; 2008).

In a general synthesis, PAA was first activated with EDC/sulfo-NHS (2 mM EDC and 5 mM sulfo-NHS in MES buffer (0.05M MES, 0.5M NaCl, pH 6.0)) for 15 min followed by filtering using a sephadex G-25 pre-packed desalting column (PD-10, GE Healthcare) to remove excess reactants. Activated PAA was then combined with CS (Phosphate buffered solution (PBS, [NaCl] 0.138M), pH 7.5, 21° C.). Conjugation of CS to the PAA backbone was monitored using the fluorescamine assay as described previously. All PAA-CS solutions were placed on a rotator and mixed continuously. Polymer was then purified by extensive dialysis against PBS to remove un-reacted CS (membrane MWCO 100,000). Dialysis was monitored using the DMMB assay for glycosaminoglycans and continued for 5 days with daily PBS changes until the dialysate solution indicated minimal CS GAG concentration.

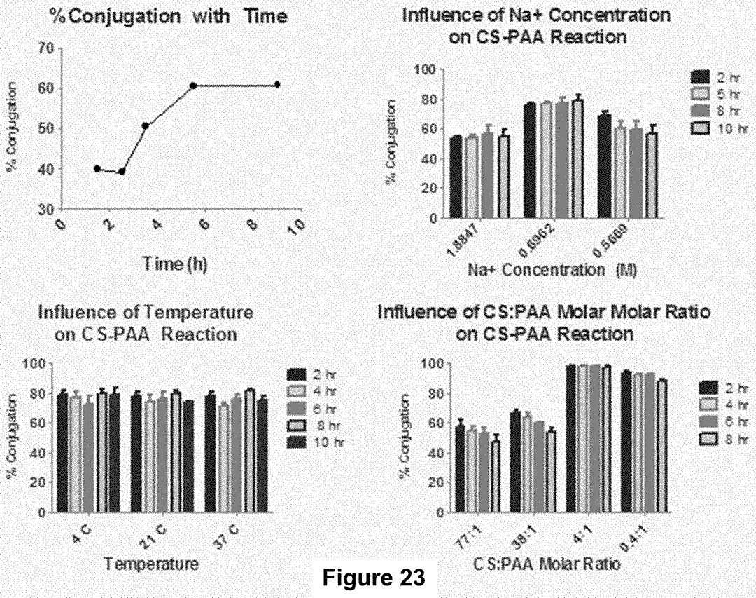

The % Conjugation of CS to PAA (33 mg/mL activated PAA, 11 mg/mL CS, PBS pH 7.5, 21° C.), increased over time with a maximum conjugation reached after 5.5 hrs (FIG. 23). The activation life of the EDC/Sulfo-NHS reaction is generally around 4 hrs caused by hydrolysis of the EDC/sulfo-NHS complex. Our results indicate that the EDC/sulfo-NHS activation life may be the rate limiting factor for achieving CS-PAA conjugates at the given reaction conditions (Hermanson G T. Bioconjugate Techniques. Second ed. Pierce Biotechnology TFS, editor. Rockford, Ill., USA: Academic Press; 2008).

Several reaction conditions were varied including solution ionic strength, reaction temperature and CS:PAA molar ratio in order to modulate and maximize attachment of CS to the PAA backbone via the carboxylic acid linking chemistry (FIG. 23). Conjugation between CS and PAA was successfully achieved and modulated by these parameters. Na+ concentration in the reaction medium affected conjugation of CS to PAA (33 mg/mL PAA, 20 mg/mL CS, pH 7.4, 21° C.) and was seen to be maximum for 0.6962 [Na+] which corresponded to buffered PBS. Utilizing this ionic formulation, the influence of temperature on the CS-PAA reaction (33 mg/mL PAA, 20 mg/mL CS, PBS, pH 7.4) was investigated and found to not significantly (p<0.05, 2-way ANOVA) effect the CS-PAA reaction. By changing the CS:PAA molar ratio (21° C., PBS, pH 7.4) CS-PAA % conjugation reached a maximum of ˜99% for reactions at a 0.4:1 CS:PAA molar ratio.

Utilizing optimized processing parameters, theoretical calculations of CS grafting density suggested approximately 46 CS chains attached to each PAA backbone resulting in one CS chain per 75 carboxylic acid sites (˜60% conjugation as determined by the fluorescamine assay for a 2 hr reaction at 77:1 CS:PAA molar ratio, PBS, pH 7.4, 21° C.). Further reaction time and CS attachment was limited by the short activity window of the EDC/sulfo-NHS activation chemistry.

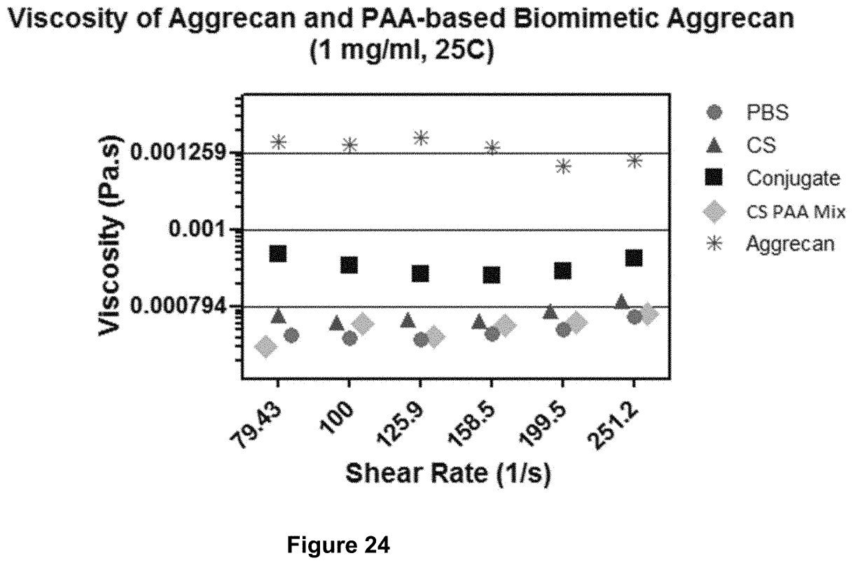

Rheological studies on the PAA-CS biomimetic aggrecan demonstrated low solution properties (viscosity of 0.871 mPa·s) compared to that of native aggrecan (1.28 mPa·s) at concentrations of 1 mg/ml although rheological properties were higher compared to that of natural CS (0.762 mPa·s) (1 mg/mL concentrations investigated in a parallel plate configuration (AR 2000ex Rheomoter), 25° C., shear rate 158/s) (FIG. 24).

The limited grafting density of the resulting PAA-CS based biomimetic aggrecan structures lead to a limited control over molecular structure and resulting physical properties however it is feasible that with further optimization and the use of other water soluable amine reactive polymeric backbones the “grafting-to” synthesis strategy may result in a family of biomimetic proteoglycans (i.e. biomimetic aggrecans and versicans etc).

### Labeling of Biomimetic Aggrecan

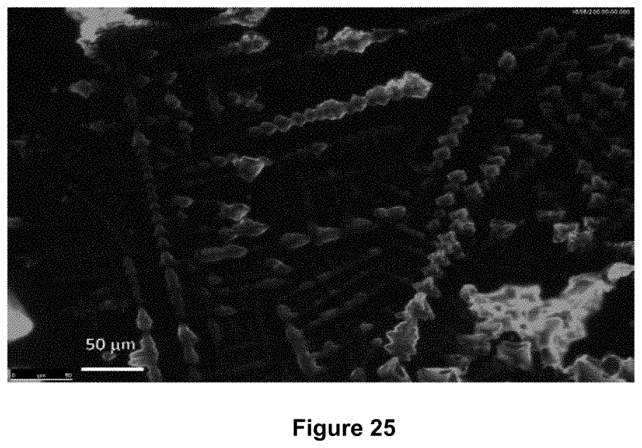

Biomimetic aggrecan (of any form discussed in this invention) may also be labeled to incorporate a marker for tracking the molecule. As an example, fluorescently label PAA-based biomimetic aggrecan was synthesized using the fluorescent hydrazide dye Alexa Fluor 488. Chondroitin sulfate has been similarly fluorescently labeled previously (Stuart et al., 2008, Biomacromolecules 10(1):25-31), however the fluorescent labeling of biomimetic aggrecan has not been previously demonstrated. Fluorescently labeled biomimetic aggrecan may allow for the monitoring of polymer distribution in cadaveric studies. The vicinal OH groups on the CS of biomimetic aggrecan were oxidized to aldehyde groups using sodium meta periodate. The oxidized CS was then reacted with the dye, whose hydrazide groups are highly reactive to the aldehyde groups. The labeled PAA-based biomimetic aggrecan was filtered using gel filtration and fluorescence was confirmed using fluorescence microscopy (CY3 fluorescent filter). The dried polymer showed strong fluorescence and took on a crystalline structure upon drying (FIG. 25). Fluorescent labeling of the biomimetic aggrecan polymer formulation was successful, and is one example of a method to tag the biomimetic aggrecan polymer.

In a general oxidation and labeling procedure equal volumes of 20 mM sodium meta periodate and 50 mg/ml of CS in 0.1M sodium acetate buffer, ph 5.5 were mixed and reacted for 1 hour at 4° C. The presence of aldehydes was tested using Schiff's reagent, which is an organic compound (rosaniline hydrochloride) that yields a magenta colored solution in the presence of an aldehyde. PAA based Biomimetic aggrecan was labeled with the Alexa fluor 488 hydrazide dye (Invitrogen), which is strongly attracted to aldehydes due to the presence of the hydrazide group. 10 mg/ml of biomimetic aggrecan was reacted with 1 mg/ml of Alexa Fluor 488 to maintain a 3-fold molar excess of dye to CS in PBS. The reaction was carried out for 2 hours at room temperature. The reaction mixture was filtered using a PD-10 column containing Sephadex G-25M medium in order to remove excess dye. The labeled biomimetic aggrecan was lyophilized and stored at −20° C.

### Example 9: Synthesis of Biomimetic Aggrecan Via Free-Radical Polymerization and Grafting-Through Strategy Utilizing the Terminal Primary Amine Handle

As an example of the “grafting-through” strategy of biomimetic aggrecan synthesis via chain growth polymerization techniques such as free-radical polymerization or anionic polymerization, the allyl glycidyl ether (AGE) based biomimetic aggrecan was further investigated. Polymeriziable CS was synthesized via the reaction of the CS terminal primary amine with the epoxide of AGE. This CS-AGE conjugate was then utilized in the free-radical polymerization of a biomimetic aggrecan macromolecule.

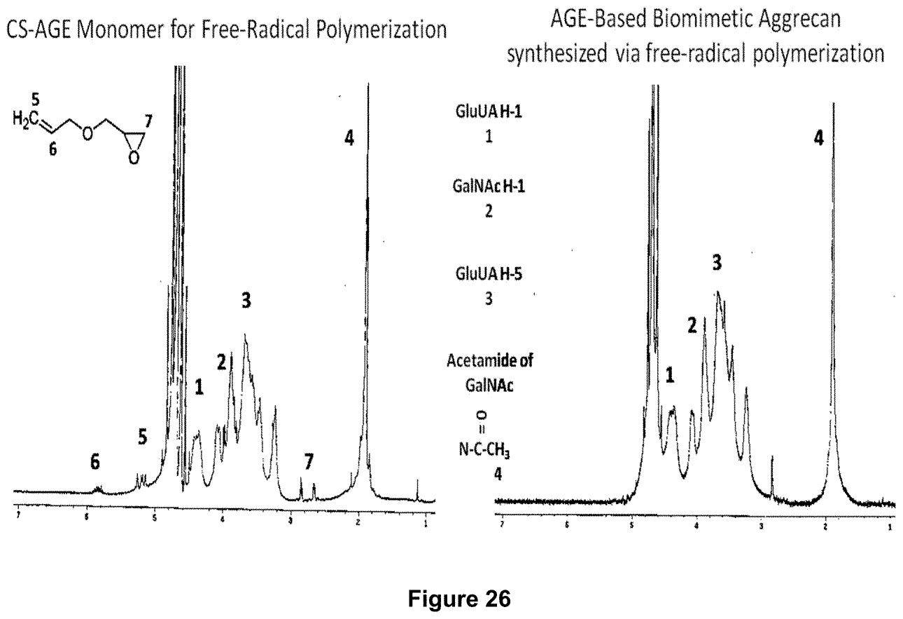

The CS-AGE conjugate described previously was utilized in the synthesis of biomimetic aggrecan via free-radical polymerization. CS-AGE conjugate was synthesized in a 20 mL volume at a CS concentration of 25 mg/mL (0.0005M) and AGE concentration of 0.5M. CS and AGE were allowed to react for 90 min at room temperature with constant mixing reaching a CS-AGE % conjugation of ˜70% as determined by the fluorescamine assay. A less than 100% conjugation of CS to AGE was targeted in order to prevent reaction of the AGE monomer to reactive groups of the CS other than the terminal primary amine. The CS-AGE conjugate was filtered to remove excess AGE via gravity filtration with the PD-10 desalting column. CS-AGE monomer solution was then placed in a 2-neck flask with a constant flow of nitrogen gas. Ammonium Persulfate was then added to the reaction mixture at 0.005M final concentration followed by TMEDA also at 0.005M final concentration. The reaction was conducted at room temperature with constant stirring for 16 hrs. ¹H-NMR analysis of the CS-AGE monomer was conducted before and after free-radical polymerization in order to monitor the reaction (FIG. 26). After polymerization, peaks corresponding to the vinyl functionality of allyl glycidyl ether were no longer present indicating successful polymerization of the AGE terminated CS.

Similarly to the example provided herein, polymerizable CS generated from but not limited to the reactions with acrylic acid and acrolein may also be polymerized into biomimetic aggrecan structures via free-radical and anionic polymerization techniques respectively.

### Example 10: Synthesis of Biomimetic Aggrecan Via the Step-Growth Grafting-Through Strategy Utilizing the Terminal Primary Amine Handle (Epoxide Linking Chemistry)

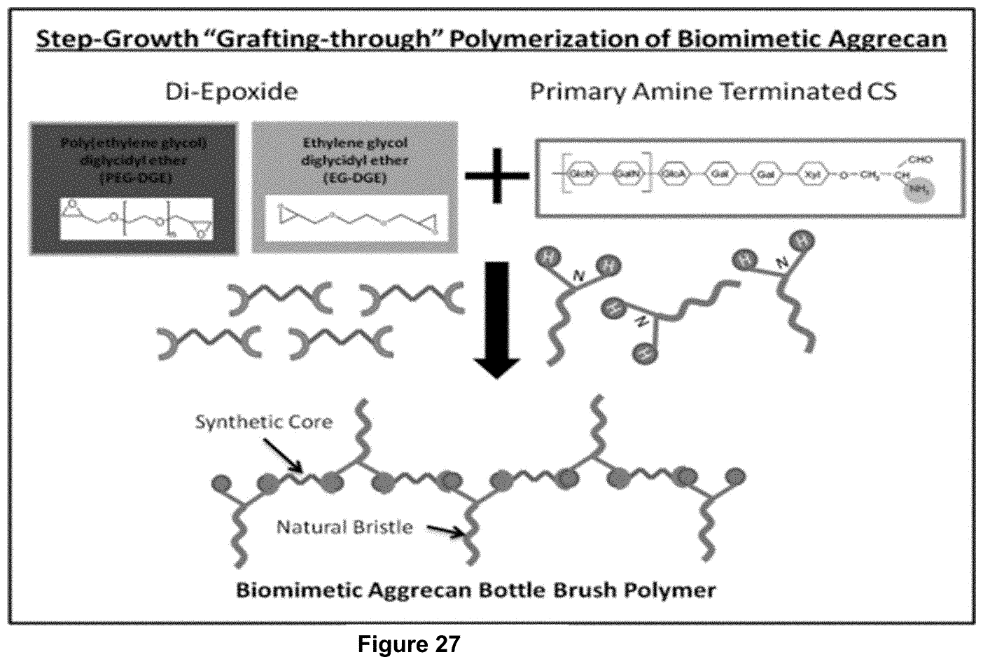

Previous studies demonstrated the facile reaction between the epoxide linking chemistry and the CS terminal primary amine handle. Utilizing this linking chemistry, biomimetic aggrecan bottle brush polymers were synthesized via the linear step-growth polymerization of di-epoxide monomers with amine terminated CS (FIG. 27) where the primary amine of each CS chain was reactive to two epoxide moieties (Swier et al., 2004, Journal Of Applied Polymer Science 91(5):2798-813; Mijovic et al., 1992, Macromolecules 25(2):979-85).

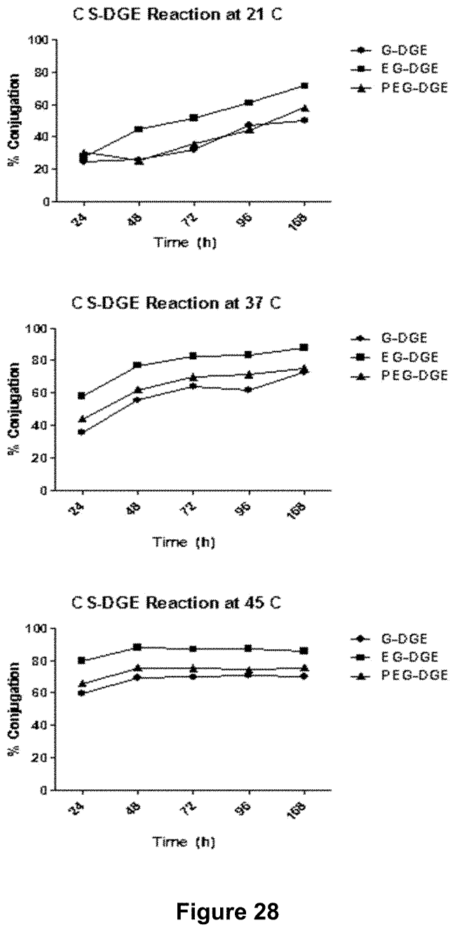

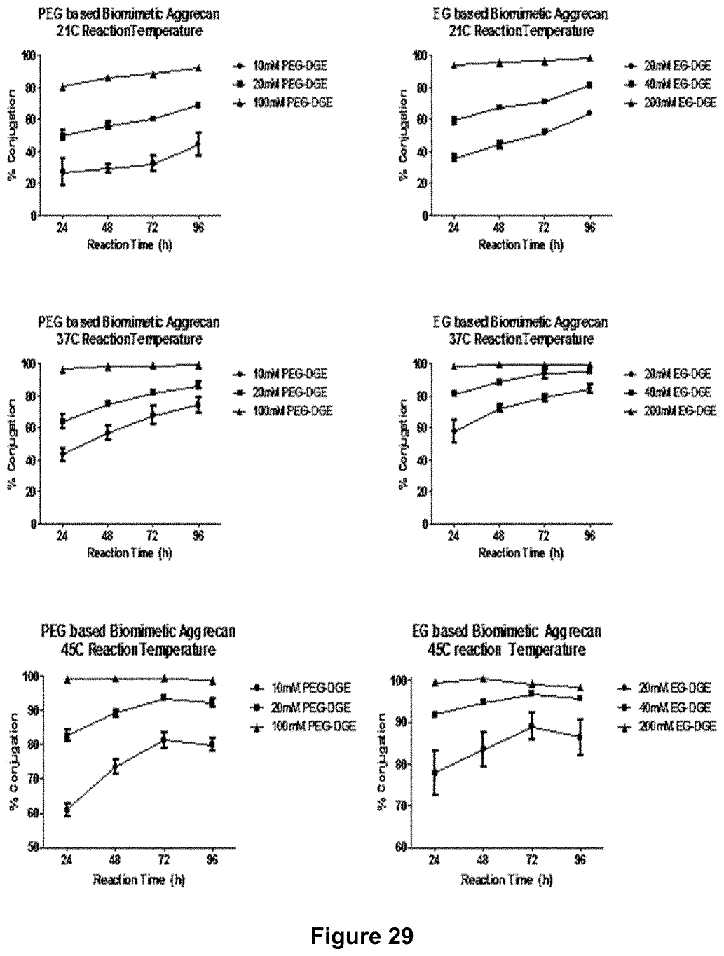

CS was reacted with several di-epoxides including glycerol diglycidyl ether (G-DGE, MW 204.2), polyethylene glycol diglycidyl ether (PEG-DGE, MW ˜526) and ethylene glycol diglycidyl ether (EG-DGE, MW 174.2) and the primary amine of CS was found to react with the di-epoxides at pH 9.4 and temperatures ranging from 21° C. to 45° C. (FIG. 28). The reaction of CS with PEG-DGE and EG-DGE were investigated further at varying temperatures and DGE concentrations (pH 9.4, 0.1M sodium borate buffer (SBB)) and resulted in the fabrication of biomimetic aggrecan polymers with polyethylene glycol or ethylene glycol synthetic polymeric cores and natural CS bristles (FIG. 29). EG based and PEG based biomimetic aggrecan have similar chemistries but differ in the molecular spacing between CS bristles (approximately 1 nm and 4 nm spacing respectively).

CS was reacted with di-epoxide (PEG-DGE or EG-DGE), and the effect of time, temperature, and di-epoxide concentration on biomimetic aggrecan synthesis was investigated (SBB, pH 9.4, CS concentration 25 mg/mL (1.4 mM), temperatures 21° C., 37° C., and 45° C., PEG-DGE concentrations of 10 mM 20 mM and 100 mM, and EG concentrations of 20 mM, 40 mM, 200 mM). CS was reacted to di-epoxides over 96 hrs and monitored for primary amine content in order to follow the reaction of the CS primary amine with the epoxides. The reaction progressed with time and was modulated by both temperature and di-epoxide concentration with reactions at 45° C. achieving the highest degree of conjugation (FIG. 29). Further purification and rheological testing were conducted on PEG and EG based biomimetic aggrecan reacted for 96 hours at 45° C., and di-epoxide concentrations of 40 mM EG-DGE or 20 mM PEG-DGE (CS concentration of 25 mg/mL). Maximum conjugation reached at these conditions was ˜96%.

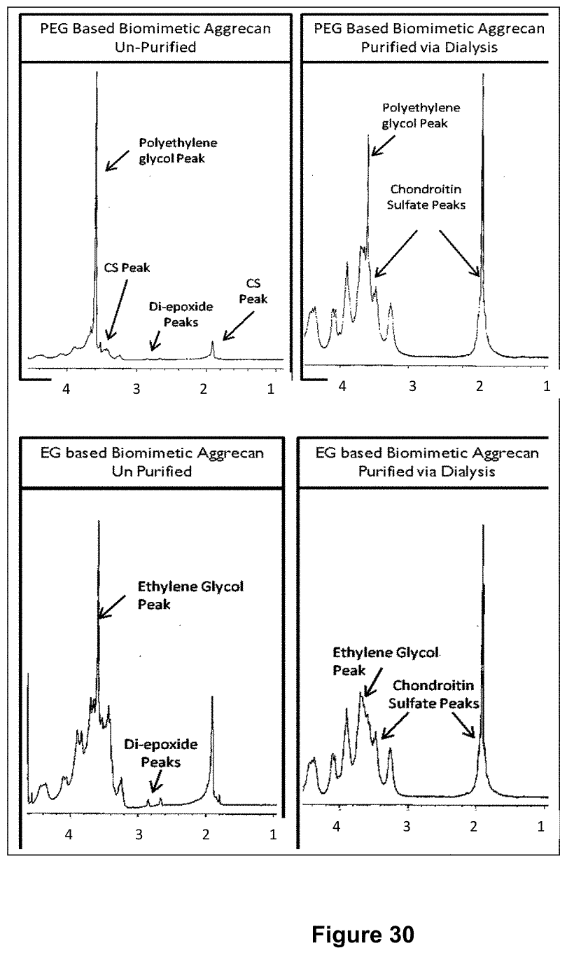

Un-reacted EG-DGE and PEG-DGE monomers were removed from the reaction by extensive dialysis (96 hrs against 1.5 L DI water, 6-8K MWCO regenerated cellulose dialysis membrane). Purification and chemical structure of the PEG and EG based biomimetic aggrecan were confirmed via ¹H-NMR (300 Mhz UnityInova NMR Spectrometer, 30 mg/mL samples in D₂O) (FIG. 30). With purification, peaks corresponding to ethylene glycol/poly(ethylene glycol) at 3.6 ppm were decreased as were peaks corresponding to epoxide groups (˜2.6 and 2.8 ppm), indicating removal of un-bound di-epoxide monomer. A peak at 3.6 ppm was still visible after removal of excess di-epoxide in PEG based biomimetic aggrecan indicating incorporation of PEG as the core of the biomimetic aggrecan polymer. For EG based Biomimetic aggrecan a small peak is seen at 3.6 ppm corresponding to the EG backbone however since EG is much smaller (MW 174.2 compared to 22,000 MW of CS) a large peak for the incorporation of EG is not expected. All peaks corresponding to CS remained similar to un-polymerized CS indicating maintenance of the CS structure in the biomimetic aggrecan polymers.

In a general synthesis procedure resulting in over 90% conjugation of CS to a diglycidyl ether, CS in reconstituted from a lyophilized state into 0.1M SBB, pH 9.4 at 25 mg/mL. The solution is mixed thoroughly to ensure a homogeneous solution. DGE is then added to the CS solution at a particular molar concentration (i.e. 10, 20, 40 100, or 200 mM). The solutions are then mixed thoroughly and placed into a 45° C. water bath with continuous shaking for 96 hrs. Samples are then assayed for conjugation using the fluorescamine assay. 20 mL of sample are then loaded into 6K-8K MWCO dialysis membranes and dialyzed against water for 96 hrs in order to remove un-reacted DGE. Purified samples are then lyophilized, resulting in a white cotton-like powder. Typical yield from a 20 mL reaction is approximately 100 mg.

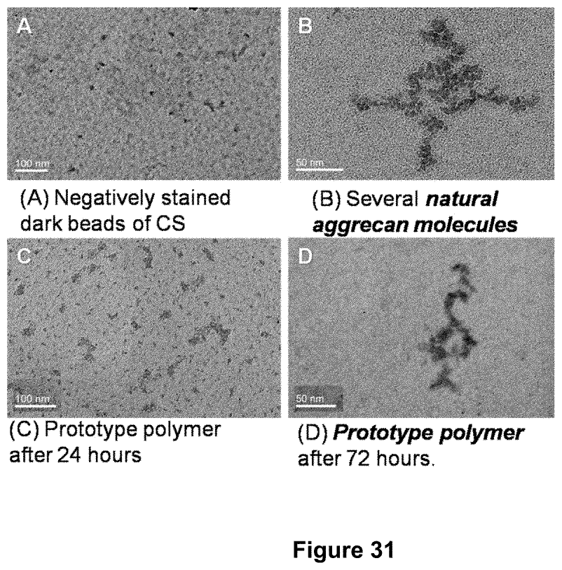

The structure of the synthesized biomimetic aggrecan was investigated using Transmission Electron Microscopy (TEM). TEM images were taken of CS, natural aggrecan, and PEG-DGE biomimetic aggrecan formulations at 24 hrs and 72 hrs of reaction (samples were mounted on copper grids and stained with uranyl acetate) (FIG. 31). CS is seen as small condensed dark spheres under TEM where the processing of samples for TEM imaging causes condensation of the CS into bead like structures. In images of aggrecan, CS can be seen arranged in a chain like structure on the protein core. After 24 hrs of reaction, the synthesized polymer appears as diffuse sphere structures. At 72 hrs, it was observed that the synthesized polymer takes on a beaded-chain like structure similar to that of native aggrecan.

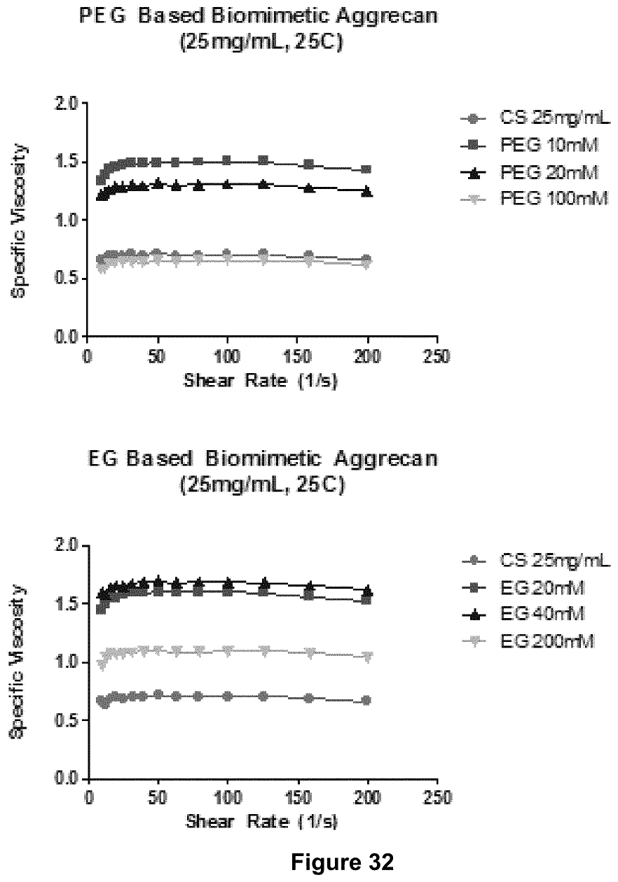

Rheological properties of the purified EG and PEG based biomimetic aggrecan were also investigated as described previously. Viscosity of the biomimetic aggrecan polymer was investigated and observed to be higher than that of CS (2.25 mPa·s for EG based biomimetic aggrecan (25 mg/mL, PBS) and 2.089 mPa·s for PEG based biomimetic aggrecan (25 mg/mL, PBS) vs 1.44 mPa·s for CS (25 mg/mL, PBS)). Specific viscosities of the EG and PEG based biomimetic aggrecans synthesized at varying PEG-DGE and EG-DGE concentrations as well as CS were determined over a range of shear rates (10-200/s) (FIG. 32). The higher specific viscosities of the biomimetic aggrecans are indicative of polymer formation and can characterize the interactions between individual polymer molecules (Waigh T, Papagiannopoulos A. Biological and Biomimetic Comb Polyelectrolytes. Polymers. 2010; 2(2):57-70; Papagiannopoulos et al., 2008, Macromolecular Chemistry and Physics 209(24):2475-86).

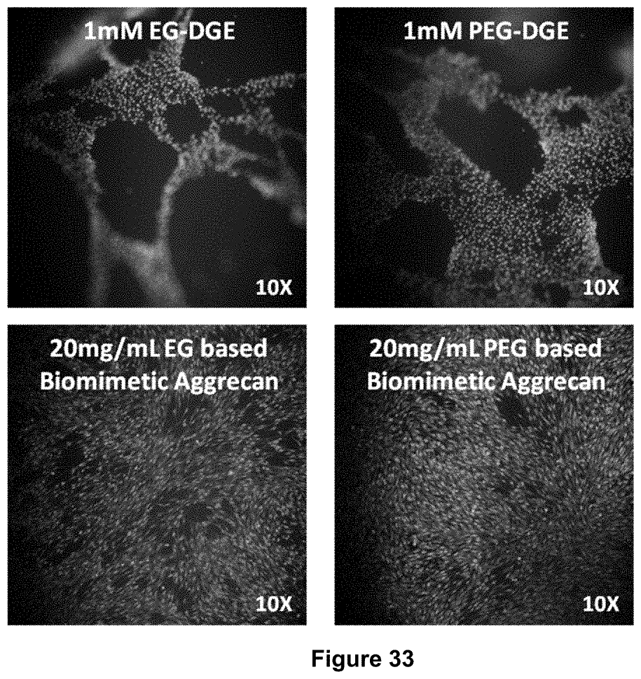

The next experiments were performed to assess the ability of the biomimetic aggrecan to support cellular growth. NIH 3T3 Fibroblasts were cultured in 48 well plates at confluent densities. Cells were allowed to attach for 24 hrs (RPMI media supplemented with 10% fetal bovine serum, L-glutamine and 1% pen/strep). Cells were then dosed with 1 mM PEG-DGE, 1 mM EG-DGE, 20 mg/mL PEG based biomimetic aggrecan or 20 mg/mL EG based biomimetic aggrecan (UV sterilized, prepared in serum supplemented media) and allowed to culture for an additional 48 hrs. Cells were similarly dosed with CS at 20 mg/mL and 70% methanol as positive and negative controls respectively (data not shown). Acute cell death was observed in both di-epoxide dosed cultures (also previously observed by Nishi et al (Nishi et al., 1995, Journal of Biomedical Materials Research 29(7):829-34)) while the majority of cells remained viable in the presence of PEG and EG based biomimetic aggrecan (FIG. 33).

An advantage of the present biomolecular design is that the engineered biomolecule is relatively resistant to enzymatic degradation while creating a hydrolytically stable molecule. In addition, without wishing to be bound by any particular theory, the biomolecules are compatible for cell viability and can support biological interactions between the biomacromolecules and nucleus pulposus cells.

A family of biomimetic aggrecan macromolecules can be synthesized using the di-epoxide linear step-growth polymerization strategy with tunable molecular weight, bristle density, and core chemistry for various soft-tissue applications.

Bristle density can be varied for a given biomimetic aggrecan MW by varying EG/PEG-DGE molecular weight (i.e. 174, 200, 400, 526, 600, and 1000 g/mol (Polysciences, Warrington, Pa.) etc.) thereby varying CS spacing for example from approximately 1-8 nm. Theoretical modeling and surface studies have predicted the magnitude of the interactions between aggrecan and CS as well as the effects of CS density on the mechanical properties of cartilaginous tissue (see section 1.3.3) (Seog et al., 2002, Macromolecules 35(14):5601-15; Han et al., 2007, Biophysical Journal 93(5):23-5; Han et al., 2007, Biophysical Journal 92(4):1384-98; Seog et al., 2005, Journal of Biomechanics 38(9):1789-97; Dean et al., 2006, Journal of Biomechanics 39(14):2555-65; Ng et al., 2003, Journal of Structural Biology 143(3):242-57; Dean et al., 2003, Langmuir 19(13):5526-39; Han et al., 2008, Biophysical Journal 95(10):4862-70). Bristle density of biomimetic aggrecan in solution is hypothesized to effect solution viscosity (Waigh T, Papagiannopoulos A. Biological and Biomimetic Comb Polyelectrolytes. Polymers. 2010; 2(2):57-70; Papagiannopoulos et al., 2008, Macromolecular Chemistry and Physics 209(24):2475-86; Papagiannopoulos et al., 2006, Biomacromolecules 7(7):2162-72; Meechai et al., 2002, Journal of Rheology 46:685) and osmotic pressure (Chahine et al., 2005, Biophysical Journal 89(3):1543-50; Kovach, 1995, Biophysical Chemistry 53(3):181-7; Bathe et al., 2005, Biophysical Journal 89(4):2357-71; Ehrlich et al., 1998, Biorheology 35(6):383-97) as well as bound water. This is because of the electrostatic interactions between closely packed CS chains and the effect of these interactions on macromolecular conformation and excluded volume (Seog et al., 2002, Macromolecules 35(14):5601-15; Seog et al., 2005, Journal of Biomechanics 38(9):1789-97; Buschmann et al., 1995, J Biomech Eng. 117(2):179-92; Baeurle et al., 2009, Polymer 50(7):1805-13; Eisenberg et al., 1985, Journal of Orthopaedic Research. 3(2):148-59).

Core chemistry can be varied by varying the di-epoxide chemical structure. Several water soluble di-epoxides with varying chemical structures can be utilized including but not limited to sorbitol polyglycidyl ether, polyglycerol polyglycidyl ether, dipropylene glycol diglycidyl ether and neopentyl glycol diglycidyl ether (Nishi et al., 1995, Journal of Biomedical Materials Research 29(7):829-34). These di-epoxides whose side chains will impart varying degrees of restriction to the rotation of the biomimetic aggrecan core limit the flexibility of the biomimetic aggrecan thereby effecting the macromolecules structural conformation and physical behavior (Waigh T, Papagiannopoulos A. Biological and Biomimetic Comb Polyelectrolytes. Polymers. 2010; 2(2):57-70).

### Example 11: Introduction of an Aldehyde into CS for Use as a Handle in Biomimetic Aggrecan Synthesis

In instances where a terminal diol or primary amine is not naturally available on a CS or other glycosaminoglycan (GAG) candidate bristle, a handle may be introduced into the bristle backbone. As an example, a single set of aldehyde groups may be introduced into the CS backbone by oxidation of a vicinal OH group on the CS using sodium meta periodate (similar to the procedures presented in example 7). In a separate body of work 1.1 aldehyde groups were introduced into dermatan sulfate and utilized for the immobilization of dermatan sulfate onto collagen (Paderi et al., 2008, Biomacromolecules 9(9):2562-6), however, this technique has not been used previously to introduce a single aldehyde reactive point onto CS for the synthesis of biomimetic aggrecan. A single set of aldehyde may be introduced onto CS using controlled oxidation (Dawlee et al., 2005, Biomacromolecules 6(4):2040-8). Although this handle may not be at the terminal end of CS it does provide a single point of attachment of the CS molecule to synthetic structures thereby producing a bottle-brush-like structure.

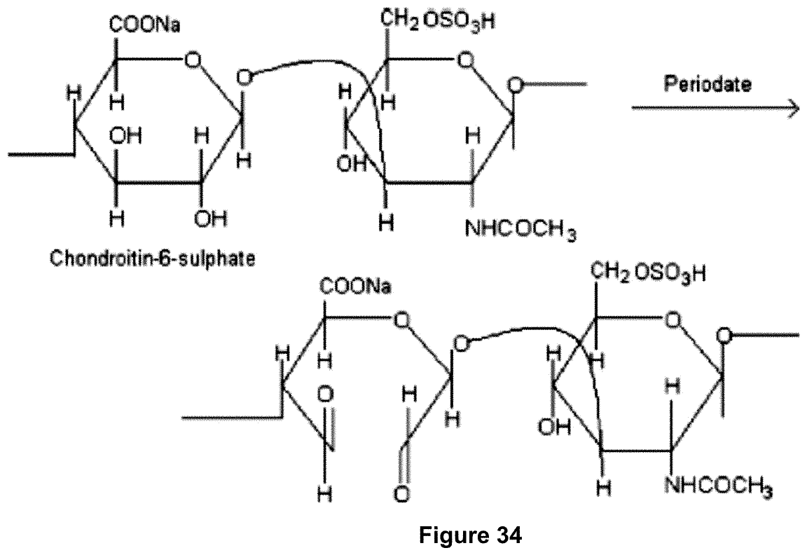

In a general procedure to achieve controlled oxidation of CS (FIG. 34), CS may be dissolved in distilled water at a concentration of 50 mg/mL then reacted with sodium meta-periodate at −4° C. for 6 hours (time may be reduced to further control oxidation). The amount of periodate is varied in order to achieve different degrees of oxidation. The extent of oxidation is determined by determining the amount of periodate remaining in the reaction mixture using iodometry. Oxidized CS is purified by dialysis against distilled water to remove excess periodate (Dawlee et al., 2005, Biomacromolecules 6(4):2040-8).

After the introduction of an aldehyde reactive group into the CS backbone, the aldehyde may be reacted with a water soluble hetero-bifunctional cross-linker such as BMPH (N-(β-maleimidopropionic acid)hydrazide.TFA, ©Pierce Biotechnology). The hydrazide of BMPH will react with the aldehyde of CS to leave an intermediate maleimide handle on CS which can be further reacted to sulfhydryl linking chemistries.

Sulfydrl chemistries may be introduced onto a poly(acrylic acid) backbone by but not limited to the reaction of PAA with cysteamine. PAA is activated by EDC/sulfo-NHS as described in elsewhere herein then added to a cysteamine hydrochloride solution with pH adjusted to between 4-5 (MES buffer). The reaction mixture is incubated for 5 hrs with constant agitation at room temperature as described elsewhere herein. The resulting PAA-cysteamine polymer is then purified by dialysis against 1 mM HCl at 10° C. Polymer may then be lyophilized and stored at 4° C. for further use (Bernkop-Schnürch et al., 2001, International Journal of Pharmaceutics 226(1-2):185-94).

In a general “grafting-to” biomimetic aggrecan synthesis, maleimide introduced CS is reconstituted in phosphate buffered solution and pH 7.4 (PBS) and allowed to react with PAA-cysteamine at room temperature where a stable thioether linkage is formed between the malemide of CS and the —SH of the PAA-cysteamine polymeric backbone.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of generating a biomimetic proteoglycan, said method comprising attaching a glycosaminoglycan (GAG) to a core structure.

2. The method of claim 1, wherein said GAG is selected from the group consisting of hyaluronic acid, chondroitin, chondroitin sulfate, heparin, heparin sulfate, dermatin, dermatin sulfate, laminin, keratan sulfate, chitin, chitosan, acetyl-glucosamine, oligosaccharides, and any combination thereof.

3. The method of claim 1, wherein said core structure is selected from the group consisting of a synthetic polymer, a protein, a peptide, a nucleic acid, a carbohydrate, and any combination thereof.

4. The method of claim 1, wherein said core structure is a synthetic polymer selected from the group consisting poly(4-vinylphenyl boronic acid), poly (3,3′-diethoxypropyl methacylate), polyacrolein, poly(N-isopropyl acrylamide-co-glycidyl methacrylate), poly(allyl glycidyl ether), poly(ethylene glycol), poly(acrylic acid), and any combination thereof.

5. The method of claim 1, wherein said GAG comprises a terminal handle selected from the group consisting of a terminal primary amine, terminal diol, and an introduced aldehyde.

6. The method of claim 1, wherein said GAG is attached to said core structure by way of a linking chemistry selected from the group consisting of a bornic acid-diol linkage, epoxide-amin linkage, aldehyde-amine linkage, carboxylic acid-amine linkage, sulfhydryl-maleimide linkage, and any combination thereof.

7. The method of claim 1, wherein said biomimetic proteoglycan has a shape selected from the group consisting of cyclic, linear, branched, star-shaped, comb, graft, bottlebrush, dendritic, mushroom, and any combination thereof.

8. The method of claim 1, wherein said biomimetic proteoglycan mimics natural proteoglycan selected from the group consisting of aggrecan, betaglycan, decorin, perlecan, serglycin, syndecan-1, biglycan, fibromodulin, lumican, versican, neurocan, brevican, and any combination thereof.

9. The method of claim 1, wherein said biomimetic proteoglycan is biomimetic aggrecan and wherein said GAG is selected from the group consisting of chondroitin sulfate, keratin sulfate, oligosaccharides, and any combination thereof.

10. A method of treating a disease, disorder, or condition associated with a soft tissue in a mammal, the method comprising administering a composition comprising a biomimetic proteoglycan to a mammal in need thereof.

11. The method of claim 10, wherein said biomimetic proteoglycan is capable of water uptake and is further electrostatically active in said mammal.

12. The method of claim 10, wherein said soft tissue is selected from the group consisting of intervertebral disc, skin, heart valve, articular cartilage, cartilage, meniscus, fatty tissue, craniofacial, ocular, tendon, ligament, fascia, fibrous tissue, synovial membrane, muscle, nerves, blood vessel, and any combination thereof.

13. The method of claim 10, wherein the biomimetic proteoglycan is a biomimetic aggrecan.

14. The method of claim 10, wherein the composition further comprises a cell, which is optionally genetically modified.

15. The method of claim 10, wherein the composition further comprises at least one biologically active molecule.

16. The method of claim 15, wherein the biologically active molecule is a growth factor, cytokine, antibiotic, protein, anti-inflammatory agent, or analgesic.

17. The method of claim 10, wherein composition further comprises a biocompatible matrix.

18. The method of claim 17, wherein the biocompatible matrix is selected from the group consisting of calcium alginate, agarose, fibrin, collagen, laminin, fibronectin, glycosaminoglycan, hyaluronic acid, heparin sulfate, chondroitin sulfate A, dermatan sulfate, bone matrix gelatin, and any combination thereof.

19. The method of claim 10, wherein
(i) the disease, disorder, or condition is a degenerated disc and the composition is administered to the mammal by an approach selected from the group consisting of a posterior approach, a posterolateral approach, an anterior approach, an anterolateral approach, and a lateral approach;
(ii) the disease, disorder, or condition is a degenerated skin and the composition is administered to the mammal by an approach selected from the group consisting of intradermal, injection, subdermal injection, subcutaneous injection, diffusion, and implantation; or
(iii) the disease, disorder, or condition is osteoarthritis and the composition is administered to the mammal by an approach to the diarthrodial joints selected from group consisting of injection, arthroscopic implantation, and open implantation.

20. The method of claim 10, wherein the composition is administered through endplates.
